(19) United States
(12) Patent Application Publication
(10) Pub. No.: US 2005/0245520 A1
(43) Pub. Date: Nov. 3, 2005

(54) 2-Phenylpyridin-4-yl derivatives as alk5 inhibitors

(76) Inventors: Nerina Dodic, Les Ulis (FR); Francoise Jeanne Gellibert, Les Ulis (FR)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/522,969

(22) PCT Filed: Jul. 29, 2003
(86) PCT No.: PCT/EP03/08496

§ 371 Date: Jan. 31, 2005

(30) Foreign Application Priority Data
Jul. 31, 2002 (GB) 0217751.7
Jun. 24, 2003 (GB) 0314698.2

(51) Int. Cl. A61K 31/5377; A61K 31/444; C07D 413/14; C07D 43/14

(52) U.S. Cl. 514/235.5; 544/124; 546/256; 514/333

(57) ABSTRACT

This invention relates to novel 2-phenylpyridin-4-yl heterocyclyl derivatives which are inhibitors of the transforming growth factor, (“TGF”)-β signalling pathway, in particular, the phosphorylation of smad2 or smad3 by the TGF-β type I or activin-like kinase (“ALK”)-5 receptor, methods for their preparation and their use in medicine, specifically in the treatment and prevention of a disease state mediated by this pathway.

This invention relates to novel 2-phenylpyridin-4-yl heterocyclyl derivatives which are inhibitors of the transforming growth factor, (“TGF”)-β signalling pathway, in particular, the phosphorylation of smad2 or smad3 by the TGF-β type I or activin-like kinase (“ALK”)-5 receptor, methods for their preparation and their use in medicine, specifically in the treatment and prevention of a disease state mediated by this pathway.

TGF-β1 is the prototypic member of a family of cytokines including the TGF-βs, activins, inhibins, bone morphogenetic proteins and Müllerian-inhibiting substance, that signal through a family of single transmembrane serine/threonine kinase receptors. These receptors can be divided into two classes, the type I or activin like kinase (ALK) receptors and type II receptors. The ALK receptors are distinguished from the type II receptors in that the ALK receptors (a) lack the serine/threonine rich intracellular tail, (b) possess serine/threonine kinase domains that are very homologous between type I receptors, and (c) share a common sequence motif called the GS domain, consisting of a region rich in glycine and serine residues. The GS domain is at the amino terminal end of the intracellular kinase domain and is critical for activation by the type II receptor. Several studies have shown that TGF-β signalling requires both the ALK and type II receptors. Specifically, the type II receptor phosphorylates the GS domain of the type I receptor for TGF-β, ALK5, in the presence of TGF-β. The ALK5, in turn, phosphorylates the cytoplasmic proteins smad2 and smad3 at two carboxy terminal serines. The phosphorylated smad proteins translocate into the nucleus and activate genes that contribute to the production of extracellular matrix. Therefore, preferred compounds of this invention are selective in that they inhibit the type I receptor and thus matrix production.

Surprisingly, it has now been discovered that a class of novel 2-phenylpyridin-4-yl heterocyclyl derivatives function as potent and selective non-peptide inhibitors of ALK5 kinase.

According to a first aspect, the invention provides a compound of formula (I), a pharmaceutically acceptable salt, solvate or derivative thereof: 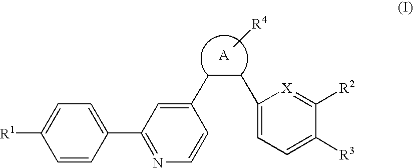
wherein
A is furan, dioxolane, thiophene, pyrrole, imidazole, pyrrolidine, pyran, pyridine, pyrimidine, morpholine, piperidine, oxazole, isoxazole, oxazoline, oxazolidine, thiazole, isothiazole, thiadiazole, benzofuran, indole, isoindole, indazole, imidazopyridine, quinazoline, quinoline, isoquinoline, pyrazole or triazole;
X is N or CH;
R¹ is hydrogen, C₁₋₆alkyl, C₁₋₆alkenyl, C₁₋₆alkoxy, halo, cyano, perfluoro C₁₋₆alkyl, perfluoroC₁₋₆alkoxy, —NR⁵R⁶, —(CH₂)ₙNR⁵R⁶, —O(CH₂)ₙOR⁷, —O(CH₂)ₙ-Het, —O(CH₂)ₙNR⁵R⁶, —CONR⁵R⁶, —CO(CH₂)ₙNR⁵R⁶, —SO₂R⁷, —SO₂NR⁵R⁶, —NR⁵SO₂R⁷, —NR⁵COR⁷, —O(CH₂)ₙCONR⁵R⁶, —NR⁵CO(CH₂)ₙNR⁶R⁶ or —C(O)R⁷;
R² is hydrogen, C₁₋₆alkyl, halo, cyano or perfluoroC₁₋₆alkyl;
R³ is hydrogen or halo;
R⁴ is hydrogen, halo, phenyl, C₁₋₆alkyl or —NR⁵R⁶;
   where
R⁵ and R⁶ are independently selected from hydrogen; Het; C₃₋₆cycloalkyl optionally substituted by C₁₋₆alkyl; or by C₁₋₆alkyl optionally substituted by Het, alkoxy, cyano or —NR^{a}R^{b} (where R^{a} and R^{b} which may the same or different are hydrogen or C₁₋₆alkyl, or R^{a} and R^{b} together with the nitrogen atom to which they are attached may form a 4,5 or 6-membered saturated ring); or R⁵ and R⁶ together with the nitrogen atom to which they are attached form a 3, 4, 5, 6 or 7-membered saturated or unsaturated ring which may contain one or more heteroatoms selected from N, S or O, and wherein the ring may be further substituted by one or more substituents selected from halo (such as fluoro, chloro, bromo), cyano, —CF₃, hydroxy, —OCF₃, C₁₋₆alkyl and C₁₋₆alkoxy;
R⁷ is selected from hydrogen and C₁₋₆alkyl;
Het is a 5 or 6-membered C-linked heterocyclyl group which may be saturated, unsaturated or aromatic, which may contain one or more, heteroatoms selected from N, S or O and which may be substituted by C₁₋₆alkyl; and
n is 1-4;
   with the provisos that:
a) when A is thiazole (wherein the thiazole sulfur is on the same side as the 4-pyridyl moiety); X is N; R¹ is hydrogen, C₁₋₆-alkyl, C₁₋₆alkoxy, halo, cyano, perfluoroC₁₋₆alkyl or perfluoroC₁₋₆alkoxy; R² is hydrogen, C₁₋₆alkyl, halo, cyano or perfluoroC₁₋₆-alkyl; and R³ is hydrogen or halo; then R⁴ is not NH₂; and
b) when X is N, A is pyrazole (where the ring containing X is attached to the pyrazole ring at carbon atom next to a pyrazole ring nitrogen) and R² is hydrogen then R³ is not hydrogen.

The term “C₁₋₆alkyl” as used herein, whether on its own or as part of a group, refers to a straight or branched chain saturated aliphatic hydrocarbon radical of 1 to 6 carbon atoms, unless the chain length is limited thereto, including, but not limited to methyl, ethyl, n-propyl, isopropyl, n-butyl, sec-butyl, isobutyl, tert-butyl, pentyl and hexyl.

The term “alkenyl” as a group or part of a group refers to a straight or branched chain mono- or poly-unsaturated aliphatic hydrocarbon radical containing the specified number(s) of carbon atoms. References to “alkenyl” groups include groups which may be in the E- or Z-form or mixtures thereof.

The term “alkoxy” as a group or part of a group refers to an alkyl ether radical, wherein the term “alkyl” is defined above. Such alkoxy groups in particular include methoxy, ethoxy, n-propoxy, iso-propoxy, n-butoxy, iso-butoxy, sec-butoxy and tert-butoxy.

The term “perfluoroalkyl” as used herein includes compounds such as trifluoromethyl.

The term “perfluoroalkoxy” as used herein includes compounds such as trifluoromethoxy.

The terms “halo” or “halogen” are used interchangeably herein to mean radicals derived from the elements chlorine, fluorine, iodine and bromine.

The term “heterocyclyl” as used herein includes cyclic groups containing 5 to 7 ring-atoms up to 4 of which may be hetero-atoms such as nitrogen, oxygen and sulfur, and may be saturated, unsaturated or aromatic. Examples of heterocyclyl groups are furyl, thienyl, pyrrolyl, pyrrolinyl, pyrrolidinyl, imidazolyl, dioxolanyl, oxazolyl, thiazolyl, imidazolyl, imidazolinyl, imidazolidinyl, pyrazolyl, pyrazolinyl, pyrazolidinyl, isoxazolyl, isothiazolyl, oxadiazolyl, triazolyl, thiadiazolyl, pyranyl, pyridyl, piperidinyl, dioxanyl, morpholino, dithianyl, thiomorpholino, pyridazinyl, pyrimidinyl, pyrazinyl, piperazinyl, sulfolanyl, tetrazolyl, triazinyl, azepinyl, oxazepinyl, thiazepinyl, diazepinyl and thiazolinyl. In addition, the term heterocyclyl includes fused heterocyclyl groups, for example benzimidazolyl, benzoxazolyl, imidazopyridinyl, benzoxazinyl, benzothiazinyl, oxazolopyridinyl, benzofuranyl, quinolinyl, quinazolinyl, quinoxalinyl, dihydroquinazolinyl, benzothiazolyl, phthalimido, benzofuranyl, benzodiazepinyl, indolyl and isoindolyl.

Preferably, A is furan, thiophene, pyrrole, imidazole, pyridine, pyrimidine, oxazole, isoxazole, thiazole, isothiazole, thiadiazole, imidazopyridine, pyrazole or triazole; each of which is optionally substituted by one or more of the substituents R⁴.

More preferably, A is triazole, imidazopyridine, thiazole, imidazole or pyrazole; each of which is optionally substituted by one or more of the substituents R⁴.

Still more preferably A is imidazopyridine, thiazole or imidazole; each of which is optionally substituted by one R⁴ substitutent.

Yet more preferably A is imidazole optionally substituted by one R⁴ substitutent.

Preferably X is N.

Preferably R¹ is C₁₋₆alkyl, C₁₋₆alkoxy, halo, cyano, perfluoroC₁₋₆alkoxy, —NR⁵R⁶, —(CH₂)ₙNR⁵R⁶, —O(CH₂)ₙOR⁷, —O(CH₂)ₙ-Het, —O(CH₂)ₙNR⁵R⁶, —CONR⁵R⁶, —SO₂R⁷, —NR⁵SO₂R⁷, —NR⁵COR⁷, —O(CH₂)ₙCONR⁵R⁶, —NR⁵CO(CH₂)ₙNR⁵R⁶or —C(O)R⁷.

More preferably R¹ is C₁₋₆alkoxy, halo, perfluoroC₁₋₆alkoxy, —NR⁵R⁶, —(CH₂)ₙNR⁵R⁶, —O(CH₂)ₙOR⁷, —O(CH₂)ₙ-Het, —O(CH₂)ₙNR⁵R⁶, —CONR⁵R⁶, —SO₂R⁷ or —O(CH₂)ₙCONR⁵R⁶.

Preferably R² is hydrogen, C₁₋₆alkyl or fluoro. More preferably R² is hydrogen or methyl. More preferably still, R² is methyl.

Preferably R³ is hydrogen.

Preferably, when X is N, R² is methyl. More preferably when X is N and R² is methyl, R³is hydrogen.

Preferably R⁴ is hydrogen, phenyl, C₁₋₆alkyl or halo. More preferably tert-butyl, isopropyl or methyl.

Preferably R⁵ and R⁶ are independently selected from hydrogen; Het (preferably tetrahydropyranyl); C₃₋₆cycloalkyl optionally substituted by C₁₋₆alkyl; or by C₁₋₆alkyl optionally substituted by Het (preferably furyl), alkoxy, cyano or —NR^{a}R^{b} (where R^{a} and R^{b} which may the same or different are hydrogen or C₁₋₆alkyl, or R^{a} and R^{b} together with the nitrogen atom to which they are attached may form a 4, 5 or 6-membered saturated ring); or R⁵and R⁶ together with the atom to which they are attached form a morpholine, piperidine, pyrrolidine or piperazine ring, each of which may be substituted by halo (such as fluoro, chloro, bromo), cyano, —CF₃, hydroxy, —OCF₃, C₁₋₄alkyl or C₁₋₄alkoxy.

More preferably R⁵ and R⁶ are independently selected from hydrogen, Het (preferably tetrahydropyranyl) or C₁₋₆alkyl; or R⁵ and R⁶ together with the atom to which they are attached form a morpholine, piperidine, pyrrolidine or piperazine ring, each of which may be substituted by halo (such as fluoro, chloro, bromo), cyano, —CF₃, hydroxy, —OCF₃, C₁₋₄alkyl or C₁₋₄alkoxy.

It will be appreciated that the present invention is intended to include compounds having any combination of the preferred groups listed hereinbefore.

Preferably
A is imidazole;
X is N;
R¹ is C₁₋₆alkyl, C₁₋₆alkoxy, halo, cyano, perfluoroC₁₋₆alkoxy, —NR⁵R⁶, —(CH₂)ₙNR⁵R⁶, —(CH₂)ₙOR⁷, —O(CH₂)ₙ-Het (preferably imidazolyl), —O(CH₂)ₙNR⁵R⁶, —CONR⁵R⁶, —SO₂R⁷, —NR⁵SO₂R⁷, —R⁵COR⁷, —O(CH₂)ₙCONR⁵R⁶, —NR⁵CO(CH₂)ₙNR⁵R⁶ or —C(O)R⁷;
—R² is hydrogen, C₁₋₆alkyl or fluoro;
R³ is hydrogen or halo;
R⁴ is hydrogen, phenyl, C₁₋₆alkyl or halo;
R⁵ and R⁶ are independently selected from hydrogen, Het (preferably tetrahydropyranyl) or C₁₋₆alkyl; or R⁵and R⁶ together with the atom to which they are attached form a morpholine, piperidine, pyrrolidine or piperazine ring, each of which may be substituted by halo (such as fluoro, chloro, bromo), cyano, —CF₃, hydroxy, —OCF₃, C₁₋₄alkyl or C₁₋₄alkoxy;
R⁷ is selected from hydrogen and C₁₋₆alkyl;
Het is a 5 or 6-membered C-linked heterocyclyl group which may be saturated, unsaturated or aromatic, which may contain one or more heteroatoms selected from N, S or O and which may be substituted by C₁₋₆alkyl; and
n is 1-4.

Compounds of formula (I) which are of special interest as agents useful in the treatment or prophylaxis of disorders characterised by the overexpression of TGF-β are selected from the list:
4-{2-tert-Butyl-5-[6-methyl]-pyridin-2-yl-1H-imidazol4-yl}-2-(4-methanesulfonyl-phenyl)-pyridine (Example 84);
4-{4-[4-(2-tert-Butyl-5-{6-methyl)pyridin-2-yl-1H-imidazol-4-yl)-pyridin-2-yl]-phenyl}-morpholine (Example 86);
N-(tetrahydropyran-4-yl)-4-(4-{2-isopropyl-5-[6-methyl-pyridin-2-yl]-1H-imidazol-4-yl}-pyridin-2-yl)-benzamide (Example 96);
4-{4-[4-(2-isopropyl-5-{6-methyl}-pyridin-2-yl-1H-imidazol-4-yl)-pyridin-2-yl]-phenyl}-morpholine (Example 97);
4-(4-{4-[2-Isopropyl-5-(6-methyl-pyridin-2-yl)-1H-imidazol-4-yl]-pyridin-2-yl}-benzyl)-dimethyl-amine (Example 105);
4-(4-{4-[2-Isopropyl-5-(6-methyl-pyridin-2-yl)-1H-imidazol-4-yl]-pyridin-2-yl}-benzyl)-morpholine (Example 104);,
N-(tetrahydropyran-4yl)-4-(4-{2-tert-Butyl-5-[6-methyl-pyridin-2-yl]-1H-imidazol4-yl}-pyridin-2-yl)-benzamide (Example 81);
(4-{4-[2-tert-Butyl-5-(6-methyl-pyridin-2-yl)-1H-imidazol-4-yl]-pyridin-2-yl}-benzyl)-pyrrolidine (Example 103);
4-(2-tert-Butyl-5-{6-methyl}-pyridin-2-yl-1H-imidazol-4-yl)-2-[4-(2-pyrrolidin-1-yl-ethoxy)-phenyl]-pyridine (Example 108); and
4-{4-[4-(2-methyl-5-{6-methyl)pyridin-2-yl-1H-imidazol-4-yl)-pyridin-2-yl]-phenyl}-morpholine (Example 98);
   and pharmaceutically acceptable salts, solvates and derivatives thereof.

For the avoidance of doubt, unless otherwise indicated, the term substituted means substituted by one or more defined groups. In the case where groups may be selected from a number of alternative groups, the selected groups may be the same or different.

For the avoidance of doubt, the term independently means that where more than one substituent is selected from a number of possible substituents, those substituents may be the same or different.

As used herein the term “pharmaceutically acceptable derivative” means any pharmaceutically acceptable salt, solvate, ester or amide, or salt or solvate of such ester or amide, of the compound of formula (I), or any other compound which upon administration to the recipient is capable of providing (directly or indirectly) the a compound of formula (I) or an active metabolite or residue thereof, e.g., a prodrug. Preferred pharmaceutically acceptable derivatives according to the invention are any pharmaceutically acceptable salts, solvates or prodrugs.

Suitable pharmaceutically acceptable salts of the compounds of formula (I) include acid salts, for example sodium, potassium, calcium, magnesium and tetraalkylammonium and the like, or mono- or di-basic salts with the appropriate acid for example organic carboxylic acids such as acetic, lactic, tartaric, malic, isethionic, lactobionic and succinic acids; organic sulfonic acids such as methanesulfonic, ethanesulfonic, benzenesulfonic and p-toluenesulfonic acids and inorganic acids such as hydrochloric, sulfuric, phosphoric and sulfamic acids and the like. Some of the compounds of this invention may be crystallised or recrystallised from solvents such as aqueous and organic solvents. In such cases solvates may be formed. This invention includes within its scope stoichiometric solvates including hydrates as well as compounds containing variable amounts of water that may be produced by processes such as lyophilisation.

Hereinafter, compounds, their pharmaceutically acceptable salts, their solvates and polymorphs, defined in any aspect of the invention (except intermediate compounds in chemical processes) are referred to as “compounds of the invention”.

The compounds of the invention may exist in one or more tautomeric forms. All tautomers and mixtures thereof are included in the scope of the present invention.

Compounds of the invention may exist in the form of optical isomers, e.g. diastereoisomers and mixtures of isomers in all ratios, e.g. racemic mixtures. The invention includes all such forms, in particular the pure isomeric forms. The different isomeric forms may be separated or resolved one from the other by conventional methods, or any given isomer may be obtained by conventional synthetic methods or by stereospecific or asymmetric syntheses.

Since the compounds of the invention are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions; these less pure preparations of the compounds should contain at least 1%, more suitably at least 5% and preferably from 10 to 59% of a compound of the invention.

Compounds of the invention may be prepared, in known manner in a variety of ways. In the following reaction schemes and hereafter, unless otherwise stated R¹ to R⁷, X and n are as defined in the first aspect. These processes form further aspects of the invention.

Throughout the specification, general formulae are designated by Roman numerals (I), (II), (III), (IV) etc. Subsets of these general formulae are defined as (Ia), (Ib), (Ic) etc . . . (IVa), (IVb), (IVc) etc.

Pyrazole Derivatives (IA)

Compounds of formula (IAa) may be prepared according to reaction scheme 1A from compounds of formula (IIA). Compounds of formula (IIA) are reacted with boron-containing compounds of formula (IIIA) using Suzuki coupling conditions (see Miyaura et al. Chem. Rev. 1995, 95: 2457) to give compounds of formula (IVA). Preferably reaction is carried out in the presence of a suitable base such as sodium carbonate, potassium carbonate, potassium hydroxide or sodium hydroxide, in the presence of a palladium or nickel catalyst, preferably at elevated temperature for a period of between 30 minutes and 48 hours. Preferred catalysts include tetrakis(triphenlyphosphine) palladium(0), palladium(II) acetate, dichlorobis(triphenylphosphine) palladium(II), tris(dibenzylideneacetone) dipalladium(0) and dichlorobis(triphenylphosphine) nickel. Compounds of formula (IVA) may be deprotected under acidic conditions (preferably hydrochloric acid) to give compounds of formula (IAa).

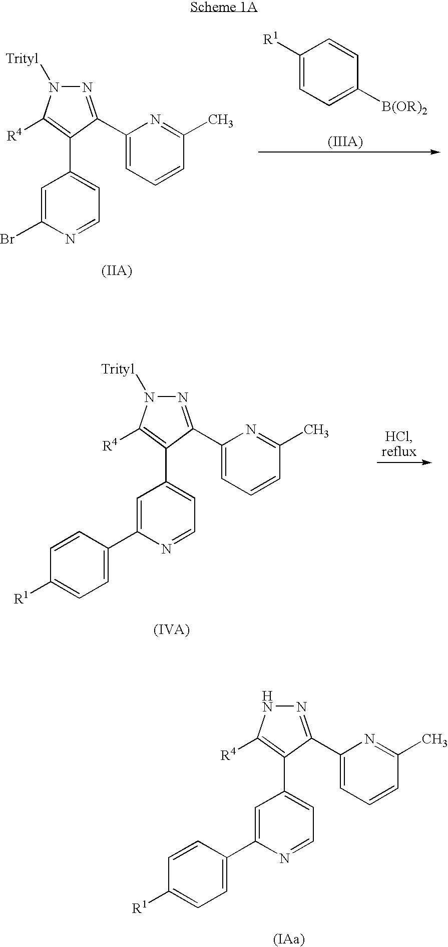

Compounds of formula (IAb) may be prepared according to reaction scheme 2A from compounds of formula (VA), by reacting compounds of formula (V(A) with dimethylformamide dimethyl acetal and acetic acid in a solvent such as DMF at room temperature, followed by treatment with hydrazine.

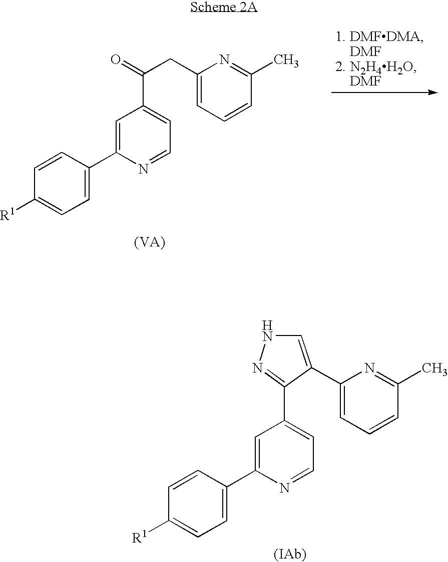

Compounds of formula (VA) may be prepared using Suzuki coupling methodology (see reaction scheme 1A) from compounds of formula (VIA) according to reaction scheme 3A. Compounds of formula (VIA) may in turn be prepared in two steps from 2-bromo-4-pyridinecarboxylic acid.

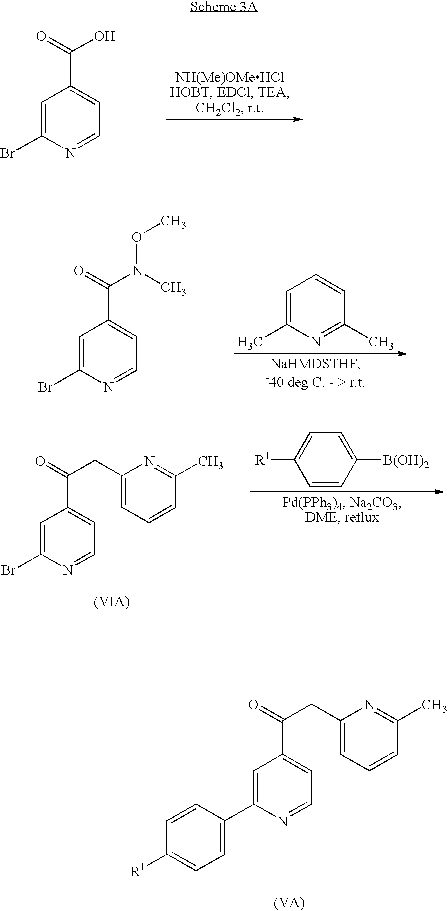

Compounds of formula (IVAa), i.e. compounds of formula (IVA) (see reaction scheme 1A) where R¹ is OR (where R is C₁₋₆alkyl, —(CH₂)ₙOR⁷, —(CH₂)ₙNR⁵R⁶ or —(CH₂)ₙHet), may be prepared from compounds of formula (VIIA) according to reaction scheme 4A, by reaction with RX (where X is a leaving group such as halogen) in the presence of base such as potassium carbonate or sodium hydride in a solvent such as dimethylformamide.

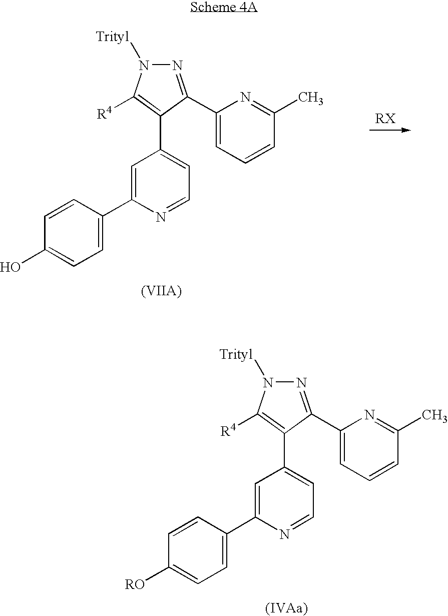

Compounds of formula (IVAb), i.e. compounds of formula (IVA) (see reaction scheme 1A) where R¹ is CONR⁵R⁶, may be prepared according to reaction scheme 5A, by reacting compounds of formula (VIIIA) with R⁵R⁶NH preferably in the presence of hydroxybenzotriazole and 1-[3-(dimethylamino)propyl]-3-ethylcarbodiimide hydrochloride.

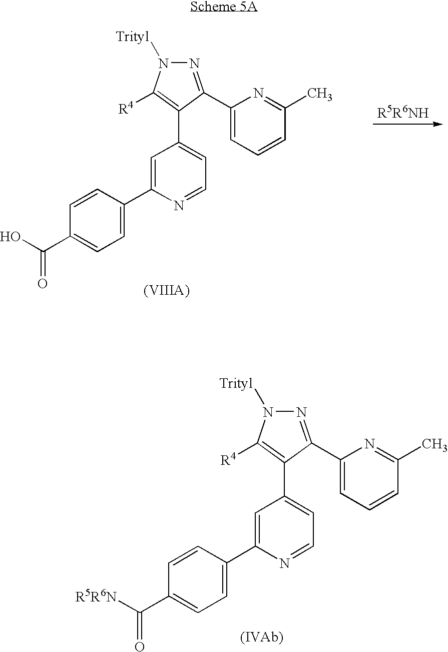

Compounds of formula (IVAc), i.e. compounds of formula (IVA) (see reaction scheme 1A) where R¹ is —CH₂NR⁶R⁶, may be prepared according to reaction scheme 6A by reacting compounds of formula (IXA) with R⁵R⁶NH in the presence of a reducing agent, preferably sodium triacetoxyborohydride in acetic acid, in a solvent such as dichloroethane at room temperature.

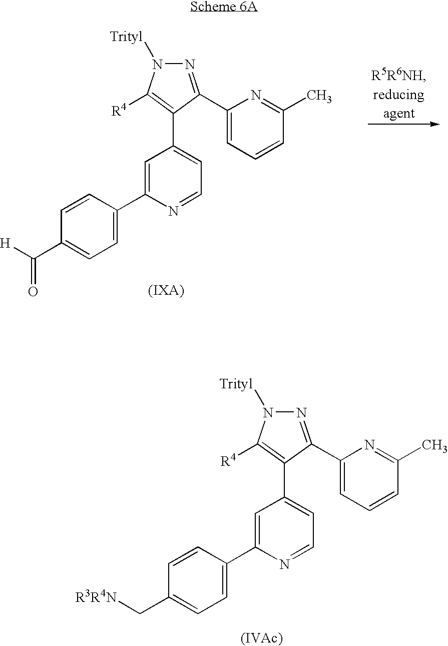

Compounds of formula (VIIA), (VIIIA) and (IXA) may be prepared by Suzuki coupling of compounds of formula (IIA) and the appropriate boron-containing compound, using conditions analogous to those described for reaction scheme 1A.

The skilled person will appreciate that compounds of formula (IVAa), (IVAb) and (IVAc) may also be prepared directly by Suzuki coupling of compounds of formula (IIA) with the appropriate boron-containing compound.

Compounds of formula (IIA) may be prepared according to reaction scheme 7A. Firstly, 2-bromo-4-methylpyridine may be coupled to compounds of formula (XA) to give compounds of formula (XIA). Preferred reaction conditions comprise treatment with a base such as sodium bis(trimethylsilyl)amide or potassium bis(trimethylsilyl)amide in tetrahydrofuran at a range of temperature from −70° C. to 0° C. Compounds of formula (XIA) may then be reacted with dimethylformamide dimethyl acetal and acetic acid in a solvent such as DMF at room temperature followed by treatment with hydrazine to give compounds of formula (XIIA) where R² is hydrogen. To prepare compounds of formula (XIIA) where R⁴ is methyl, N,N-dimethylacetamide acetal is used instead of dimethylformamide dimethyl acetal. Reaction of compounds of formula (XIIA) with trityl chloride gives compounds of formula (IIA). 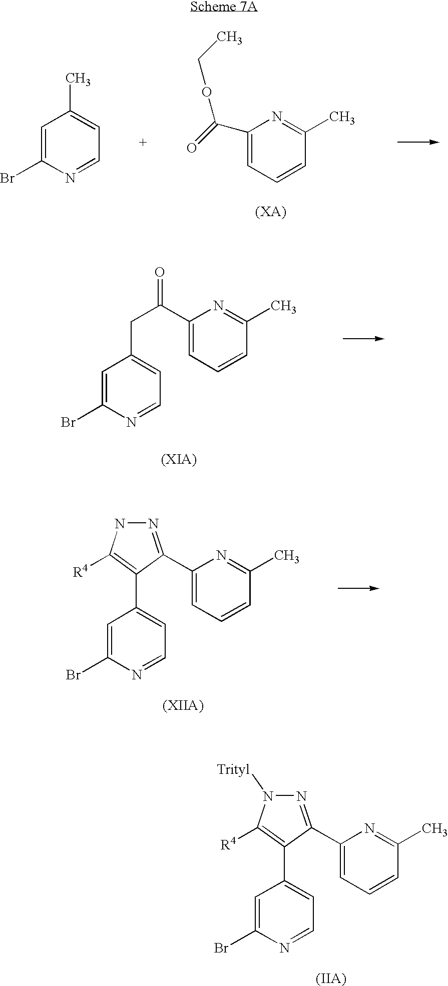
Triazole Derivatives (IB)

Compounds of formula (IB) may be prepared from compounds of formula (IIB) by treatment with an azide source according to reaction scheme 1B. Preferred reaction conditions comprise treating compounds of formula (IIB) with trimethylsilylazide at elevated temperature in a suitable solvent such as dimethylformamide.

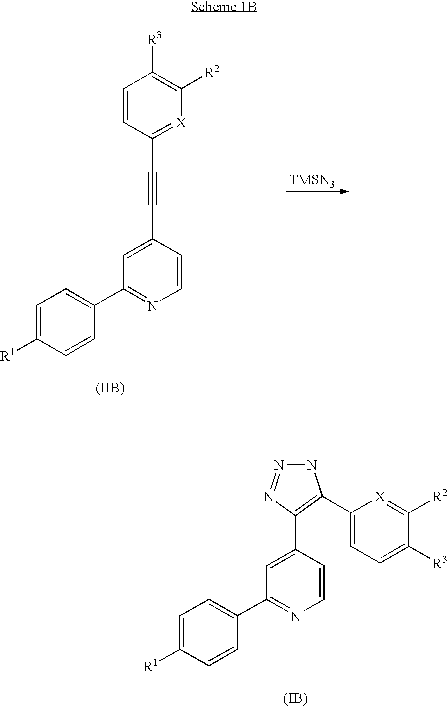

Compounds of formula (IIB) may be prepared by reacting compounds of formula (IIIB) (where Y is a leaving group such as halogen preferably chlorine) with boronic acid derivatives of formula (IVB) according to reaction scheme 2B. Preferred conditions are those developed by Miyaura et al (Chem. Rev. 1995, 95: 2457), typically comprising reaction inert solvent in the presence of a base and a palladium or nickel catalyst at a temperature of between room temperature and 130° C. for a period between 30 minutes and 48 hours. Suitable bases include sodium carbonate, potassium carbonate, potassium hydroxide, sodium hydroxide. Suitable catalysts include tetrakis(triphenlyphosphine) palladium(0), palladium(II) acetate, dichlorobis(triphenylphosphine) palladium(II), tris(dibenylideneacetone) dipalladium(0) and dichlorobis(triphenylphosphine) nickel.

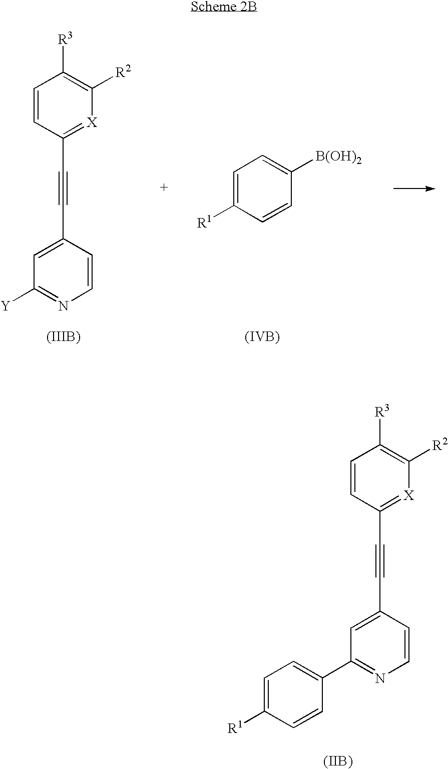

Compounds of formula (IIIB) may be prepared by Sonagashira coupling of compounds of formula (VB) (where preferably Y is chlorine and Z is iodine) with compounds of formula (VIB) according to reaction scheme 3. Preferred reaction conditions comprise reaction in an inert solvent in the presence of a base and a palladium catalyst at a temperature of between room temperature and 80° C., for a period of between 30 minutes and 48 hours. Suitable bases include TMEDA or triethyl amine. Suitable palladium catalysts include tetrakis(triphenlyphosphine) palladium(0) and dichlorobis(triphenylphosphine) palladium(II).

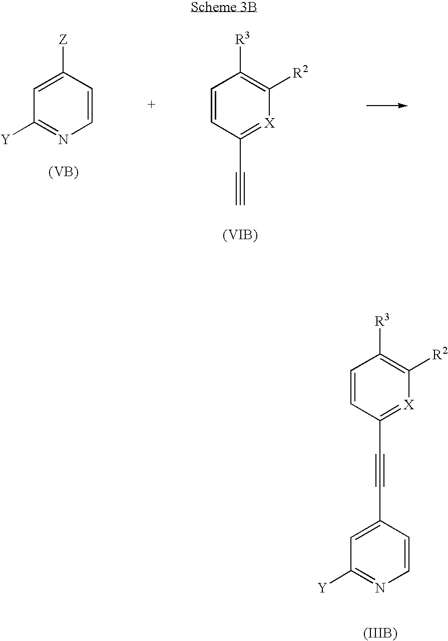

Compounds of formula (VIB) may be prepared according to reaction scheme 4B where Y¹ in compounds of formula (VIIB) is a leaving group, preferably bromine. Preferred reaction conditions for the preparation of compounds of formula (VIIIB) comprise treating compounds of formula (VIIB) with trimethylsilylacetylene in the presence of TMEDA and copper iodide under palladium catalysis in an inert solvent such as tetrahydrofuran at elevated temperature. The trimethylsilyl group may be removed by treating compounds of formula (VIIIB) with a base such as potassium carbonate in a protic solvent such as methanol.

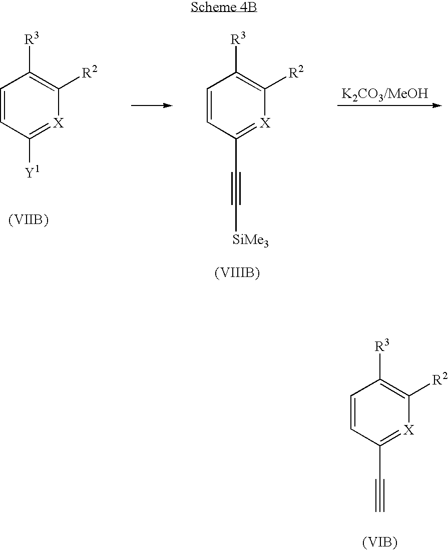

Compounds of formula (IIBa), i.e. compounds of formula (IIB) where R¹ is —O(CH₂)₂NR⁵R⁶, may be prepared from compounds of formula (IIIB) (where Y is preferably chlorine) according to reaction scheme 5B. Compounds of formula (IIIB) may be reacted with compounds of formula (IXB) to give compounds of formula (IlBa) in one step. Alternatively compounds of formula (IIIB) may firstly be reacted with 4-hydroxy-phenyl boronic acid, followed by alkylation with R⁵R⁶N(CH₂)₂Cl in the presence of a base such as potassium carbonate or sodium hydride in a solvent such as dimethylformamide.

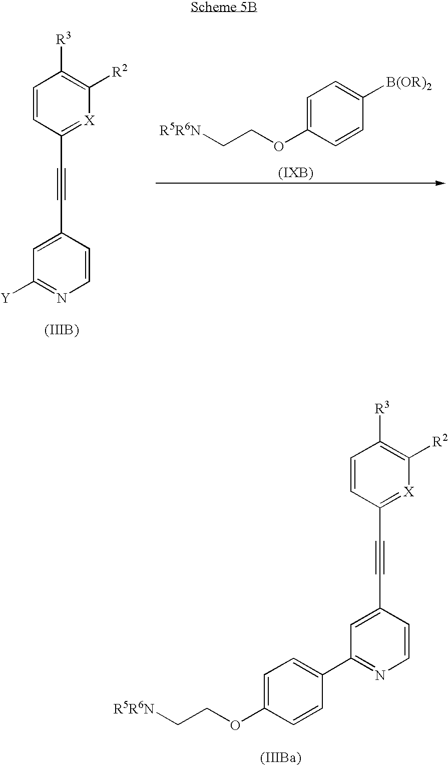

Compounds of formula (IIBb), i.e. compounds of general formula (IIB) where R¹ is —CH₂NR⁵R⁶, may be prepared according to reaction scheme 6B. Compounds of formula (IIIB) (where Y is preferably chlorine) may be reacted with 4-formylphenyl boronic acid using analogous conditions to reaction scheme 2 to give compounds of formula (XB). Compounds of formula (XB) may then be reacted with R⁵R⁶NH in the presence of a reducing agent, such as sodium cyanoborohydride in acetic acid at room temperature, to give compounds of formula (IIBb). 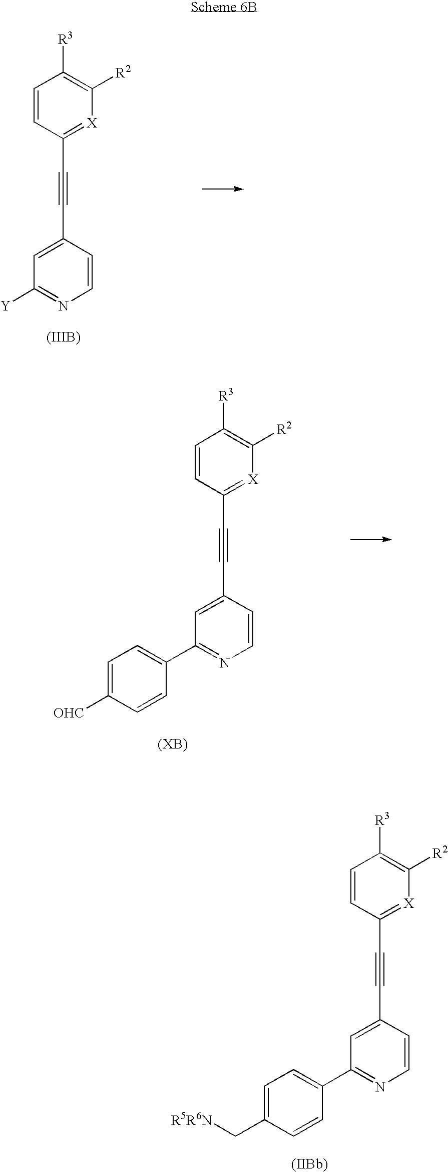
Imidazopyridine Derivatives (IC)

Compounds of formula (IC) may be prepared from compounds of formula (IIC) according to reaction scheme 1C, by reacting compounds of formula (IIC) with compounds of formula (IIIC). Preferred reaction conditions comprise boron coupling of compounds of formula (IIIC) where Y is —B(OH)₂ or 4,4,5,5-tetramethyl-[1,3,2]-dioxaborolan-2-yl cyclic derivative, with a compound of formula (IIC) in the presence of a suitable palladium catalysis (preferably Pd(PPh₃)₄) and a suitable base (preferably sodium carbonate) in an inert solvent (preferably 1,2-dimethoxyethane) at elevated temperature.

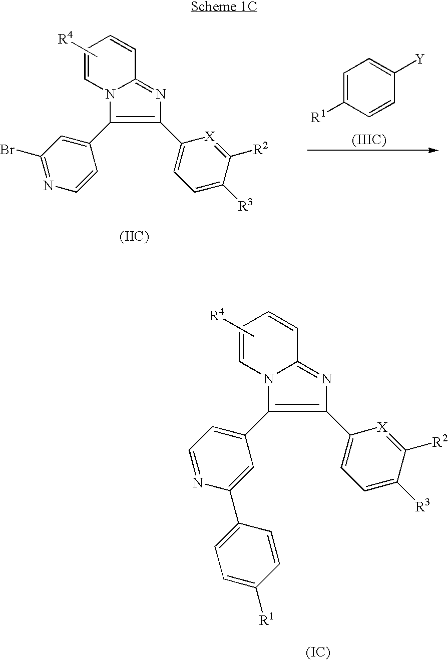

Compounds of formula (ICa), i.e. compounds of formula (IC) where R¹ is —CH₂NR⁵R⁶, may be prepared by reductive amination of compounds of formula (IVC) according to reaction scheme 2C. Preferred reaction conditions comprise reacting (IVC) with HNR⁵R⁶ in the presence of NaHB(OAc)₃, in a suitable solvent (preferably dichloromethane) at room temperature.

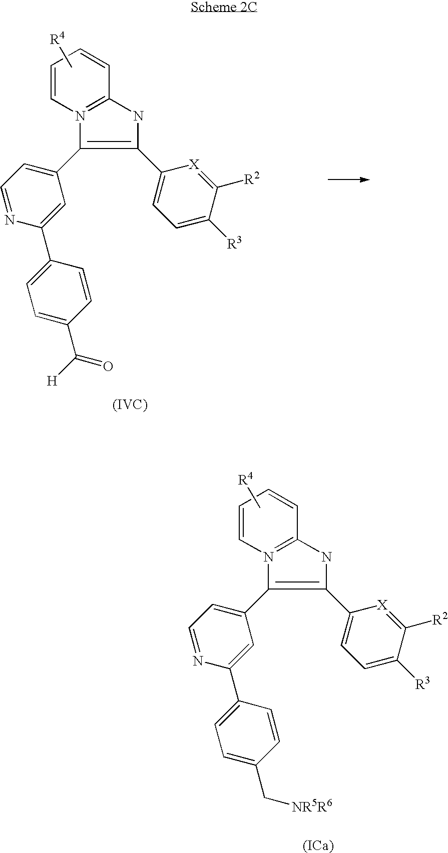

Compounds of formula (IIIC) are available from commercial sources or may be prepared by methods analogous to those described in the Examples section hereinafter.

Compounds of formula (ICb), i.e. compounds of formula (IC) where R¹ is —NR⁵R⁶, may be prepared according to reaction scheme 3C by reacting compounds of formula (ICc), i.e. compounds of formula (IC) where R¹ is bromine, with HNR⁵R⁶ in the presence of a catalyst system preferably tris(dibenzylideneacetone)dipalladium(0) and 2,2′-bis(diphenylphosphino)-1,1′-binaphthyl (Binap) in potassium tert-butoxide in a suitable solvent such as toluene at elevated temperature.

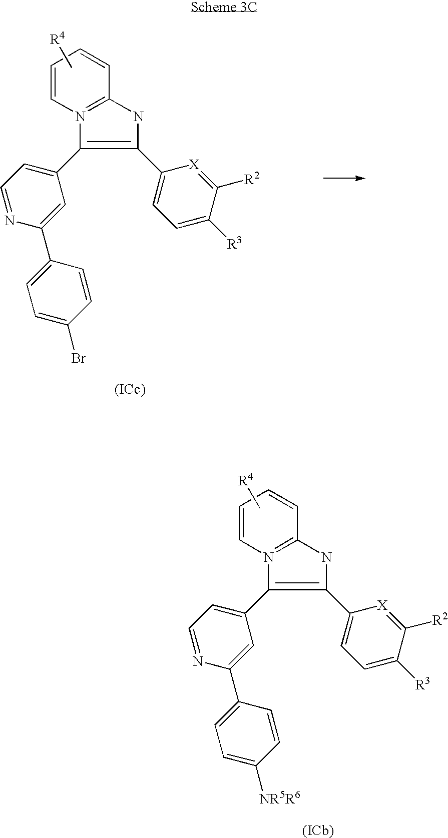

Compounds of formula (ICd), i.e. compounds of formula (IC) where R¹ is —OCH₂CH₂NR⁵R⁶, may be prepared according to reaction scheme 4C by reacting compounds of formula (VC) with 1,2-dibromoethane in the presence of a base preferably potassium carbonate in a suitable solvent, such as acetone, at elevated temperature. Treatment with HNR⁵R⁶ in a suitable solvent such as tetrahydrofuran at elevated temperature gives (ICd).

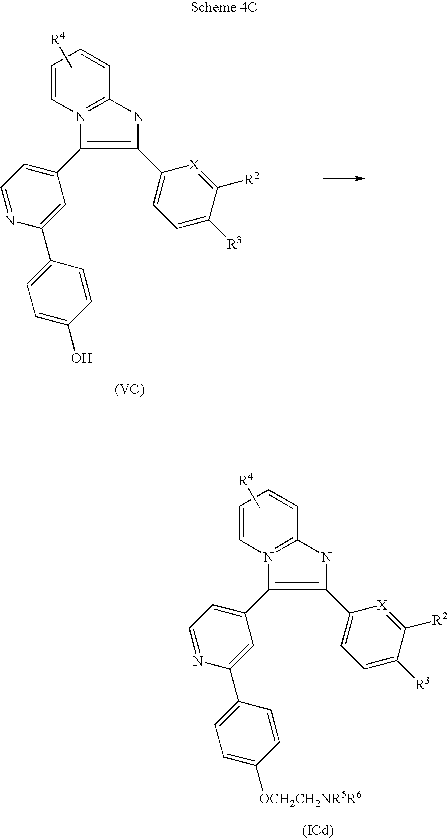

Compounds of formula (ICe), i.e. compounds of general formula (IC) where R¹ is —CONR⁵R⁶, may be prepared according to reaction scheme 5C. Compounds of formula (VIC) (where R is methyl or ethyl) are firstly saponified by heating with sodium hydroxide in methanol, followed by conversion of the resulting carboxylic acid to amide (ICe). Preferred reaction conditions comprise treating the intermediate carboxylic acid with HNR⁵R⁶ in the presence of HOBT, EDCl and a suitable base such as triethylamine in a suitable solvent such as dimethylformamide at room temperature.

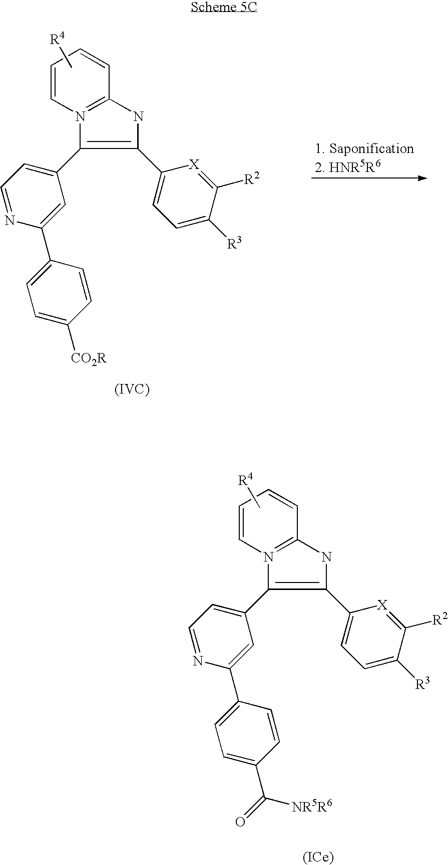

Compounds of formula (ICg), i.e. compounds of general formula (IC) where R¹ is —NHSO₂CF₃, may be prepared in two steps according to reaction scheme 6C. Firstly the acetyl group is removed from compounds of formula (ICh) by treatment with sodium hydroxide in methanol at elevated temperature. The resulting amine is then treated with CF₃SO₂Cl preferably in the presence of a base such as triethylamine in a suitable solvent such as dichloromethane at room temperature.

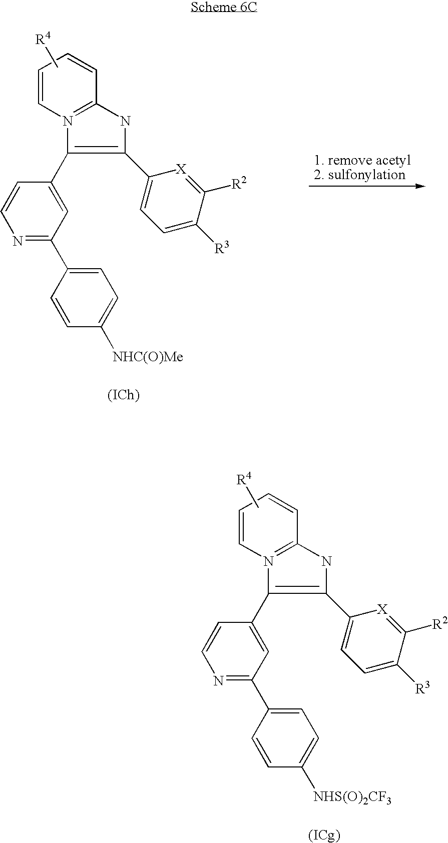

It will be apparent to the skilled person that compounds of formula (IC) may also be prepared by introducing R¹ before formation of the imidazopyridine. For instance, compounds of formula (ICi), i.e. compounds of formula (IC) where R¹ is morpholine, X is N and R³ is H may be prepared according to reaction scheme 7C.

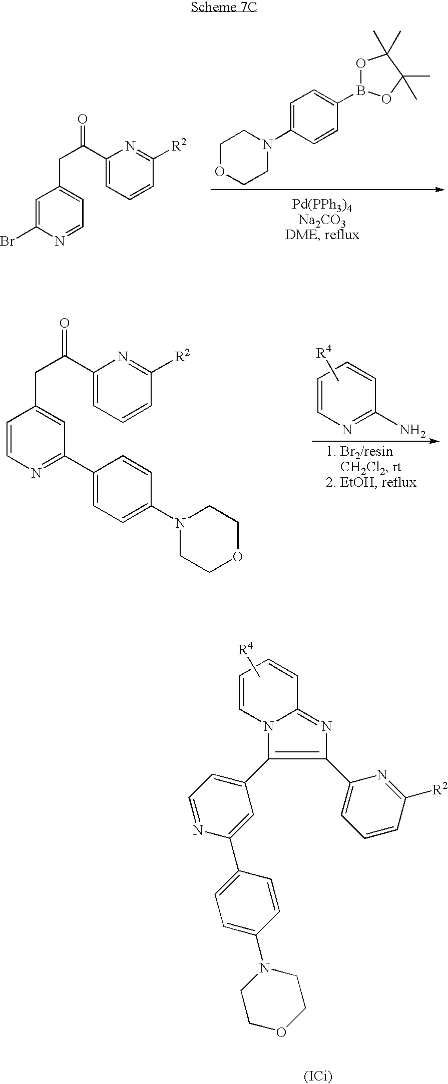

Compounds of formula (IIC) (see Scheme 1C) may be prepared in two steps according to reaction scheme 8C. Compounds of formula (VIIC) are firstly reacted with a suitable polymer-supported bromine reagent, such as polymer-supported pyridinium perbromide, in a suitable solvent such as dichloromethane at room temperature. Treatment with a compound of formula (VIIIC) in a suitable solvent such as ethanol at elevated temperature gives compounds of formula (IIC).

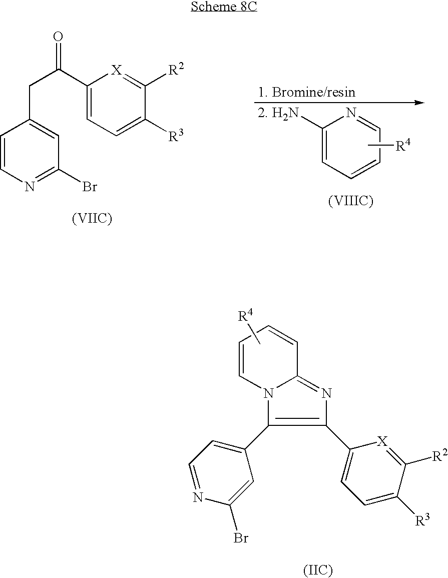

Compounds of formula (VIIC) may be prepared according to reaction scheme 9C by reacting 2-bromo-4-methylpyridine with compounds of formula (IXC) in the presence of a suitable base such as sodium bis(trimethylsilyl)amide in a suitable solvent such as tetrahydrofuran at −78° C. to −30° C. 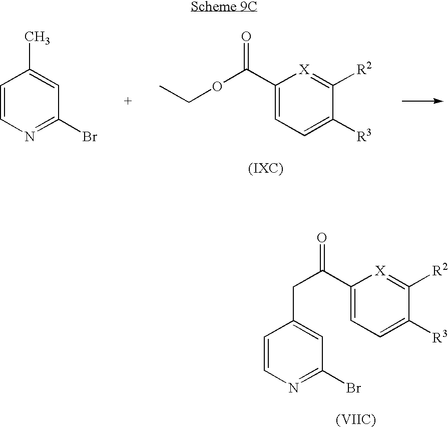
Imidazole Derivatives (ID)

Compounds of formula (ID) may be prepared according to Scheme 1ID. Compounds of formula (IID) may be treated with sodium nitrite in HCl to give compounds of formula (IIID). Compounds of formula (IIID) may then be condensed with a suitably substituted aldehyde and ammonium acetate followed by treatment with triethylphosphite to give compounds of formula (IVD) according to the method outlined in U.S. Pat. No. 5,656,644. Boronic acid coupling gives compounds of formula (ID). Preferred coupling conditions are those developed by Miyaura et al (Chem. Rev. 1995, 95: 2457), typically comprising reaction in an inert solvent in the presence of a base and a palladium or nickel catalyst at temperature between room temperature and 130° C. for a period between 30 minutes and 48 hours. Suitable bases include sodium carbonate, potassium carbonate, potassium hydroxide, sodium hydroxide. Suitable catalysts include tetrakis(triphenlyphosphine) palladium(0), palladium(II) acetate, dichlorobis(triphenylphosphine) palladium(II), tris(dibenylideneacetone) dipalladium(0) and dichlorobis(triphenylphosphine) nickel.

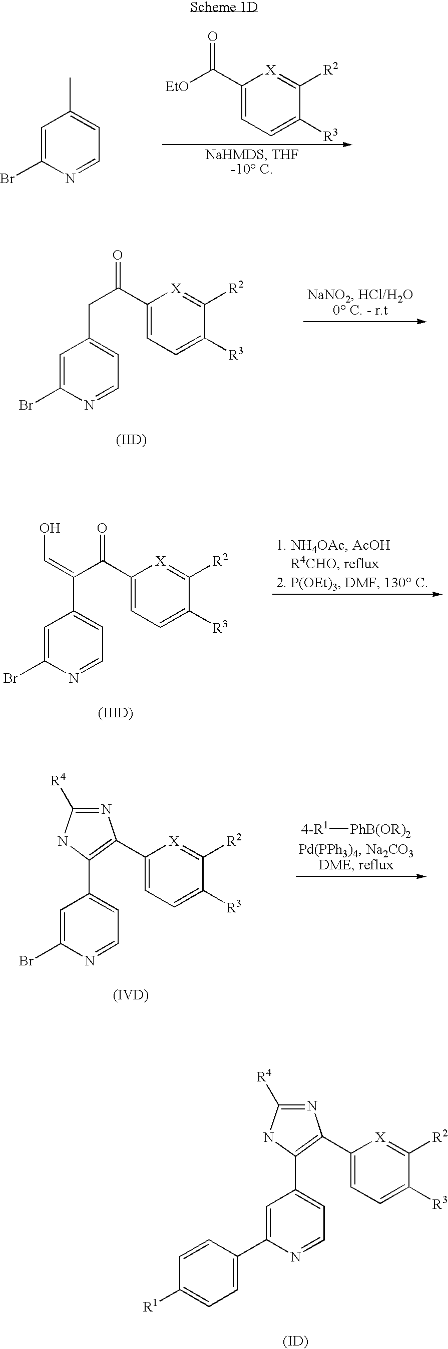

Compounds of formula (IDa), i.e. compounds of formula (I) where X is N, R¹ is —CH₂NR⁵R⁶ and R³ is hydrogen, may be prepared in one step according to scheme 2D from compounds of formula (VD).

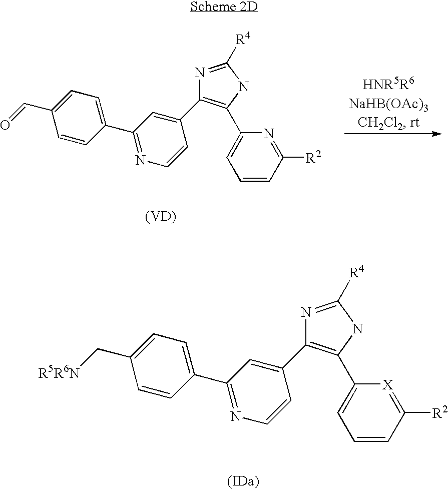

Compounds of formula (IDb), i.e. compounds of formula (ID) where X is N, R¹ is —NR⁵R⁶ and R³ is hydrogen, may be prepared according to reaction scheme 3D by reacting compounds of formula (VID) with HNR⁵R⁶ in the presence of a catalyst system preferably tris(dibenzylidene acetone)dipalladium(0) and 2,2bis(diphenylphosphino)-1,1′-binaphthyl (Binap) in potassium tert-butoxide in a suitable solvent such as toluene at elevated temperature.

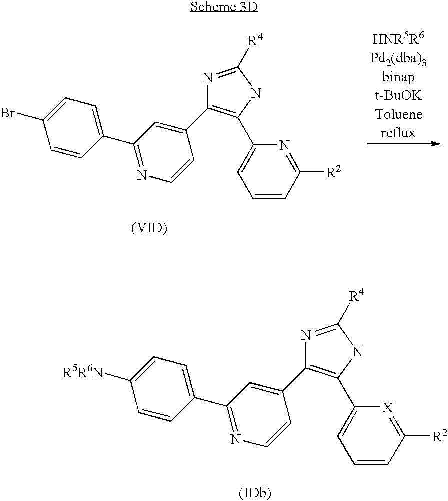

Compounds of formula (IDc), i.e. compounds of formula (ID) where X is N, R¹ is —OCH₂CH₂NR⁵R⁶ and R³ is hydrogen, may be prepared according to reaction scheme 4D by reacting compounds of formula (VIID) with 1,2-dibromoethane in the presence of a base preferably potassium carbonate in a suitable solvent, such as acetone, at elevated temperature. Treatment with HNR⁵R⁶ in a suitable solvent such as tetrahydrofuran at elevated temperature gives (IDc).

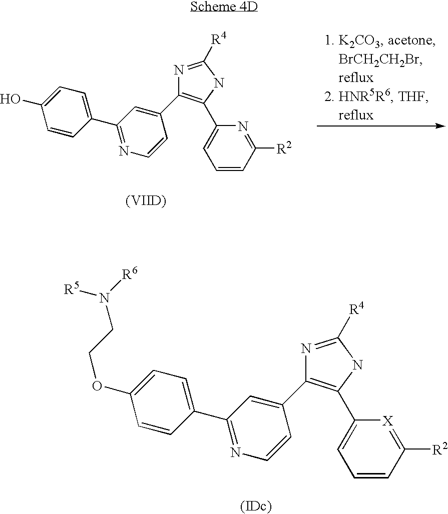

Compounds of formula (IDd), i.e. compounds of formula (ID) where X is N, R¹ is —OCH₂-Het and R³ is hydrogen, may be prepared according to reaction scheme 5D by reacting compounds of formula (VIIID) with compounds of formula (IXD) in the presence of a base (preferably sodium hydride) in a suitable solvent, such as DMF, at room temperature.

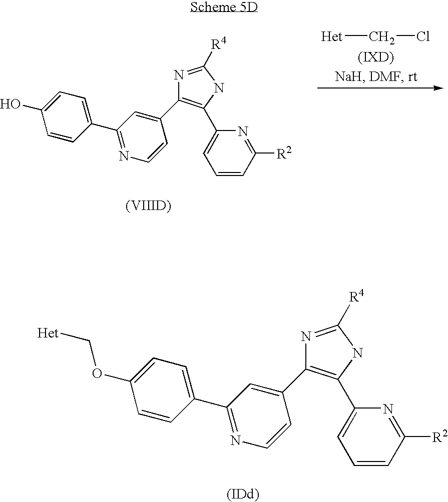

Compounds of formula (IDe), i.e. compounds of general formula (ID) where X is N, R¹ is —CONR⁵R⁶ and R³ is hydrogen, may be prepared according to reaction scheme 6D. Compounds of formula (XD) (where R is methyl or ethyl) are saponified by heating with sodium hydroxide in methanol followed by conversion of the resulting carboxylic acid to amide (IDe). Preferred reaction conditions comprise treating the intermediate carboxylic acid with HNR⁵R⁶ in the presence of hydroxybenzotriazole (HOBT), 1-[3-(Dimethylamino)propyl]-3-ethylcarbodiimide hydrochloride (EDCl) and a suitable base such as triethylamine in a suitable solvent such as dimethylformamide at room temperature.

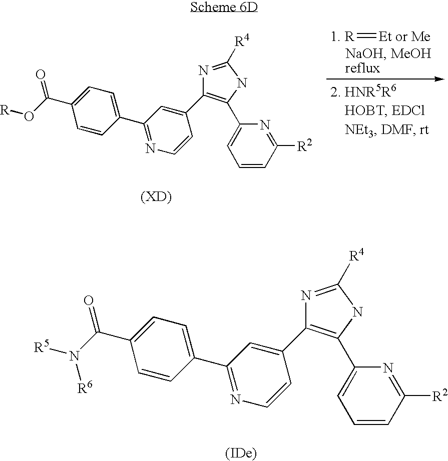

Compounds of formula (ID) where X is N and R¹ is —NHSO₂CF₃ or —NHCOCF₃, may be prepared from common intermediate of formula (XID) according to reaction scheme 7D. Firstly the acetyl group is removed from compounds of formula (XID) by treatment with sodium hydroxide in methanol at elevated temperature. The resulting amine is then treated with CF₃SO₂Cl or CF₃COCl preferably in the presence of a base such as triethylamine in a suitable solvent such as dichloromethane at room temperature. 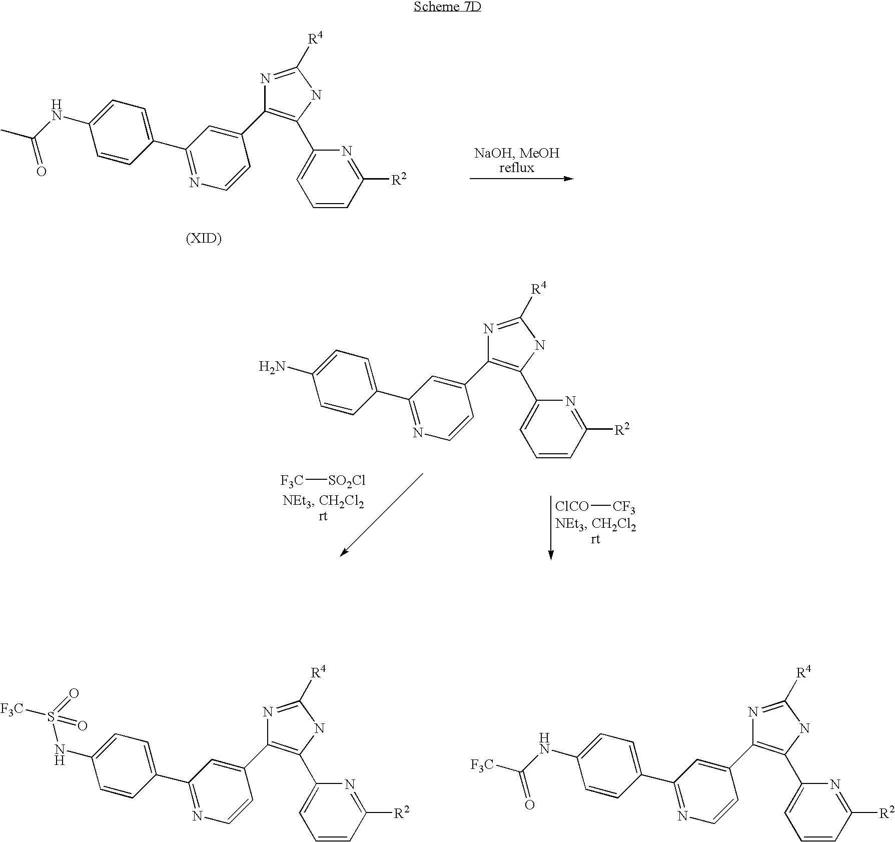
Aminothiazole Deriviatives (IE)

Compounds of formula (IEa), i.e. compounds of general formula (IE) where A is S, B is N and R⁴ is NH₂, may be prepared by reacting compounds of formula (IIE) with a suitable polymer-supported bromine reagent, such as polymer-supported pyridinium perbromide, followed by treatment with thiourea in a suitable solvent such as ethanol, preferably at elevated temperatures (see reaction scheme 1E).

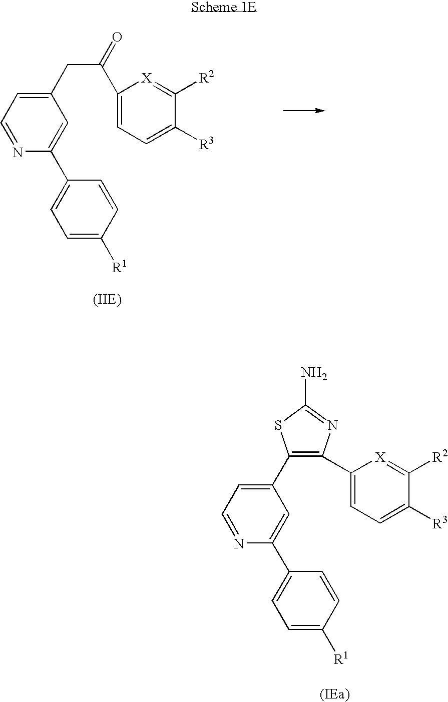

Compounds of formula (IEb), i.e. compounds of general formula (IE) where A is N, B is S and R⁴ is NH₂, may be prepared by reacting compounds of formula (IIIE) under analogous conditions to reaction scheme 1E (see reaction scheme 2E).

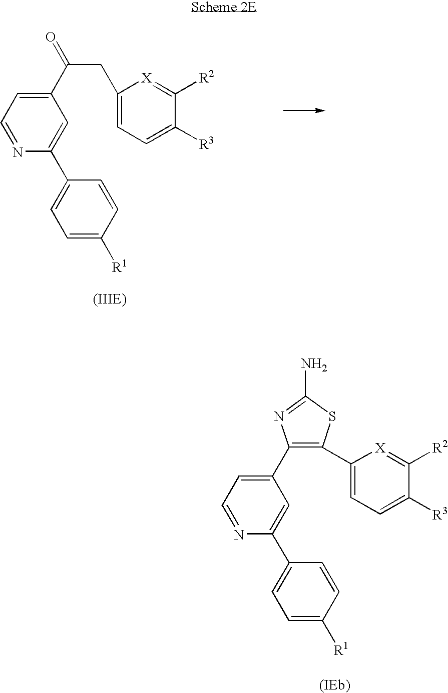

Compounds of formula (IIE) may be prepared by reacting compounds of formula (IVE) with compounds of formula (VE) where Y is a boron containing moiety such as —B(OH)₂ or 4,4,5,5-tetramethyl-1,3,2-dioxaborolan-2-yl according to reaction scheme 3E. Preferred conditions comprise reaction with a suitable catalyst such as tetrakis(triphenylphosphine) palladium (0), in the presence of a suitable base such as sodium carbonate in a suitable solvent such as DME at elevated temperature.

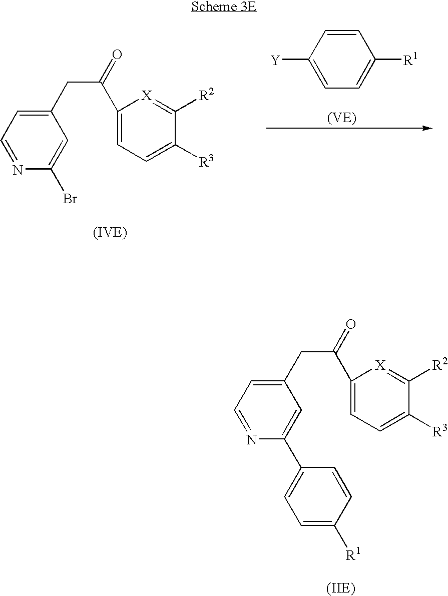

Alternatively compounds of formula (IIE) may be prepared by reacting compounds of formula (VIE) with compounds of formula (VIIE) according to reaction scheme 4E. Preferred reaction conditions comprise reacting (VIE) with sodium bis-(trimethylsilyl)amide in a suitable solvent such as tetrahydrofuran at low temperature, preferably −78° C.

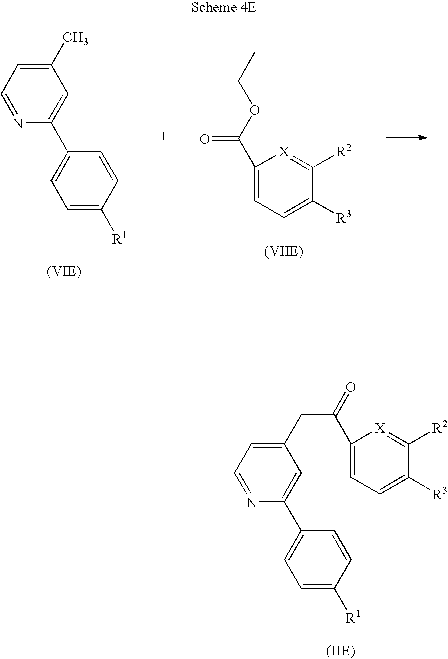

Compounds of formula (IIIE) may be prepared according to reaction scheme 5E by reacting compounds of formula (VIIIE) with compounds of formula (VE) (where Y is as defined for reaction scheme 3E) using analogous reaction conditions to those of reaction scheme 3E.

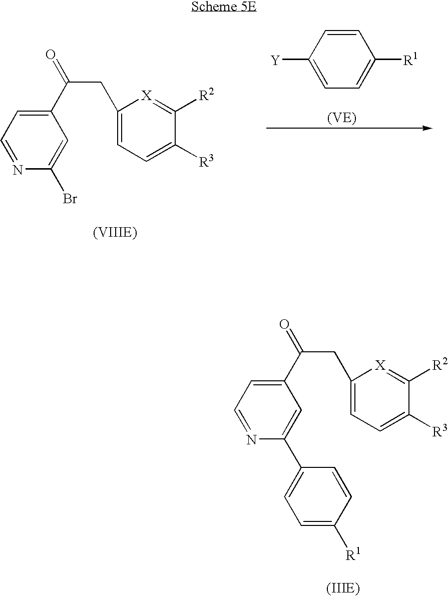

Alternatively compounds of formula (IIIE) may be prepared according to reaction scheme 6E by reacting compounds of formula (IXE) with compounds of formula (XE) in the presence of a suitable base such as cesium carbonate in a suitable solvent such as tetrahydrofuran and isopropanol at room temperature.

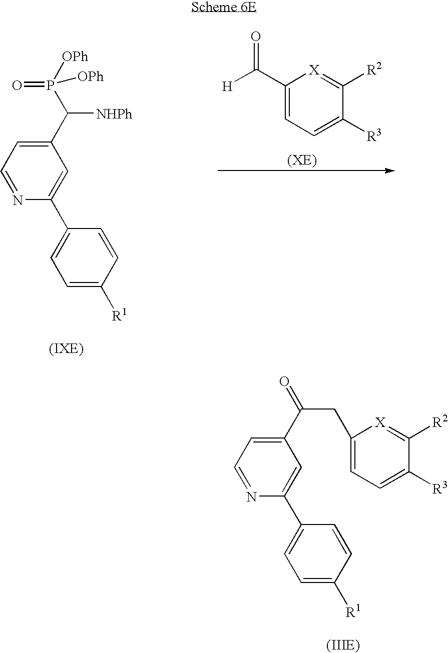

Compounds of formula (VIIIEa), i.e. compounds of general formula (VIIIE) (see scheme 5E) where X is N, R² is methyl and R³ is hydrogen, may be prepared according to reaction scheme 7E. Preferred conditions comprise reacting 2,6-lutidine with a strong base such as n-butyllithium or sodium bis-(trimethylsilyl) amide at low temperature, followed by addition of 2-bromo-N-methoxy-N-methyl4-pyridinecarboxamide.

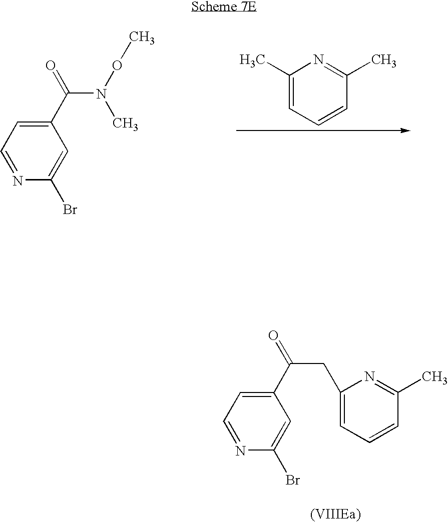

Compounds of formula (IXE) may be prepared in two steps according to reaction scheme 8E. Preferred reaction conditions for the first step are analogous to those described for reaction scheme 3E. Preferred reaction conditions for the second step comprise reacting compounds of formula (XIE) with aniline and diphenylphosphite in a suitable solvent such as isopropanol at room temperature.

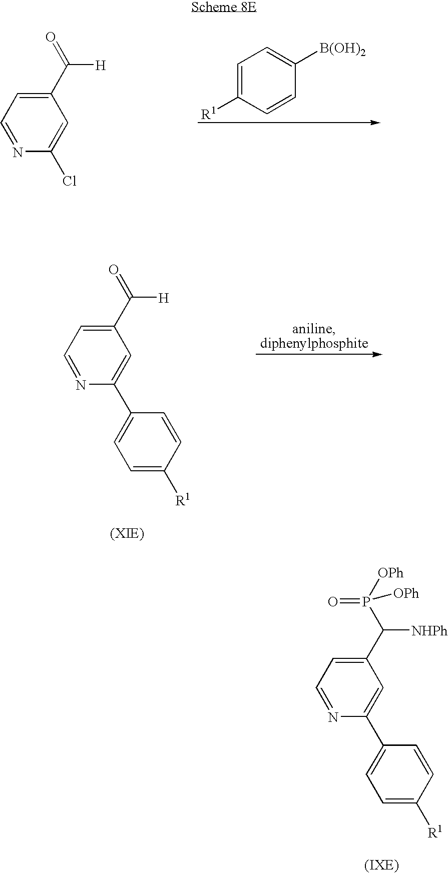

Compounds of general formula (IE) may also be prepared using solid supported chemistry.

Compounds of formula (IEc), i.e. compounds of general formula (I) where A is S, B is N, R¹ is —OR (where R is for example —(CH₂)ₙ-Het or —CH₂CONR⁵R⁶) and R⁴ is NH₂, may be prepared from solid supported compounds of formula (XIIE) by reaction with RX (where X is a suitable leaving group such as chlorine) followed by cleavage under acidic conditions from the solid support, according to reaction scheme 9E. Preferred conditions comprise treating (XIIE) with RX under basic conditions such as potassium carbonate in a suitable solvent such as DMSO at elevated temperature. Preferred cleavage conditions are trifluoroacetic acid in a suitable solvent such as dichloromethane at room temperature.

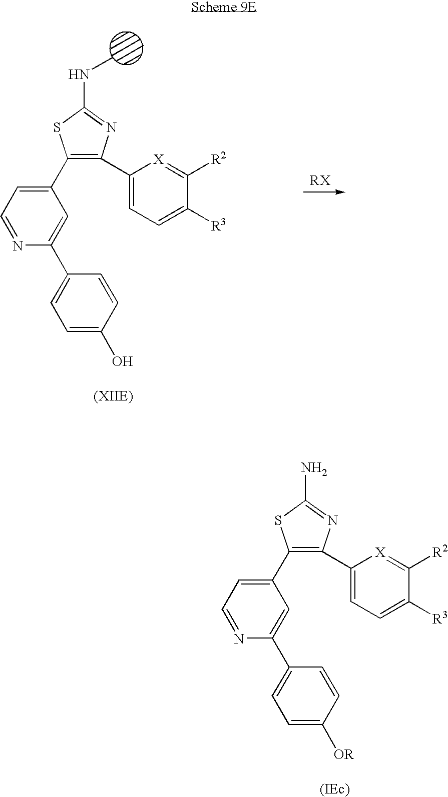

Compounds of formula (IEd), i.e. compounds of general formula (IE) where A is S, B is N, R¹ is —CH₂NR⁵R⁶ and R⁴ is NH₂, may be prepared from solid supported compounds of formula (XIIIE) according to reaction scheme 10E. Preferred reaction conditions comprise treating (XIIIE) with HNR⁵R⁶ in trimethylorthoformate and addition of a reducing agent, such as sodium cyanoborohydride in acetic acid at elevated temperature. Cleavage from the solid support using trifluoroacetic acid in dichloromethane gives compounds of formula (IEd).

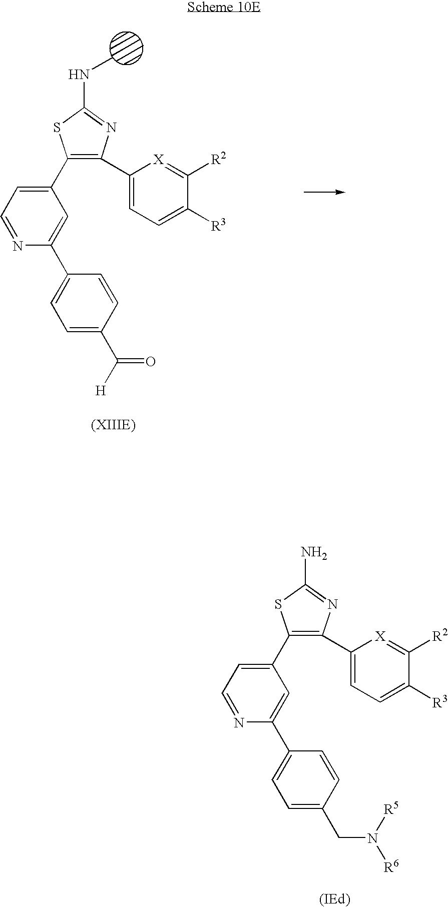

Compounds of formula (IEe), i.e. compounds of general formula (IE) where A is S, B is N, R¹ is —C(O)NR⁵R⁶ and R⁴ is NH₂, may be prepared from solid supported compounds of formula (XIVE) according to reaction scheme 11E. Preferred reaction conditions comprise treating (XIVE) with HNR⁵R⁶, hydroxybenzotriazole and diisopropylcarbodiimide. Cleavage from the solid support using trifluoroacetic acid in dichloromethane gives compounds of formula (IEe).

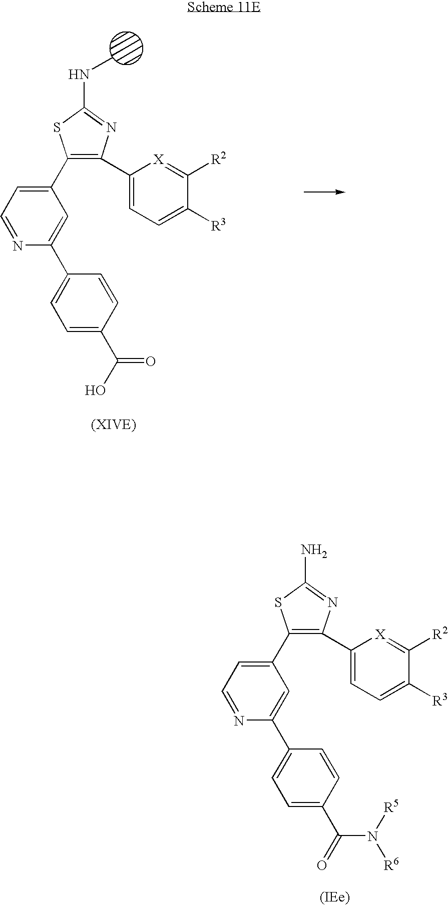

Compounds of formula (XIIE), (XIIIE) and (XIVE) may be prepared according to reaction scheme 12E from compounds of formula (XVE) and the appropriate arylboronic acid (XVIE), where Z is —OH, —CHO or —CO₂H respectively.

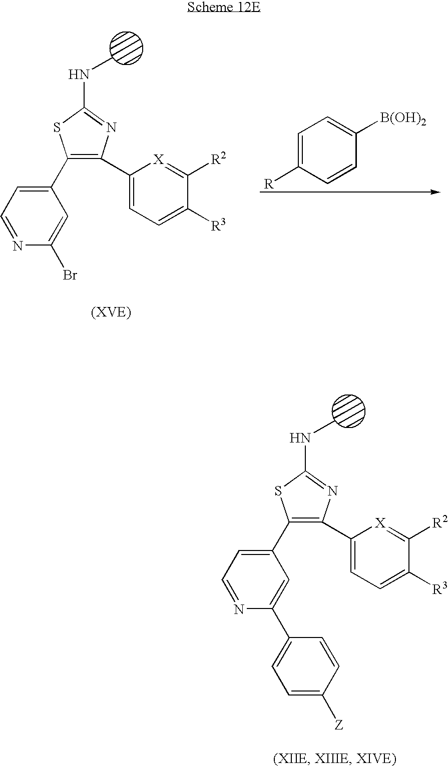

Compounds of formula (XVE) may be prepared from solid-phase synthesis according to reaction scheme 13E. Compounds of formula (XVIE) may be prepared by treating compounds of formula (IVE) (see scheme 3E) with a suitable polymer-supported bromine reagent, such as polymer-supported pyridinium perbromide. Treatment of a resin bound thiourea with a dioxane solution of compounds of formula (XVI) gives the compounds (XV) using general conditions described in the literature (Kearney P. C., J. Org. Chem., (1998), 63, 196).

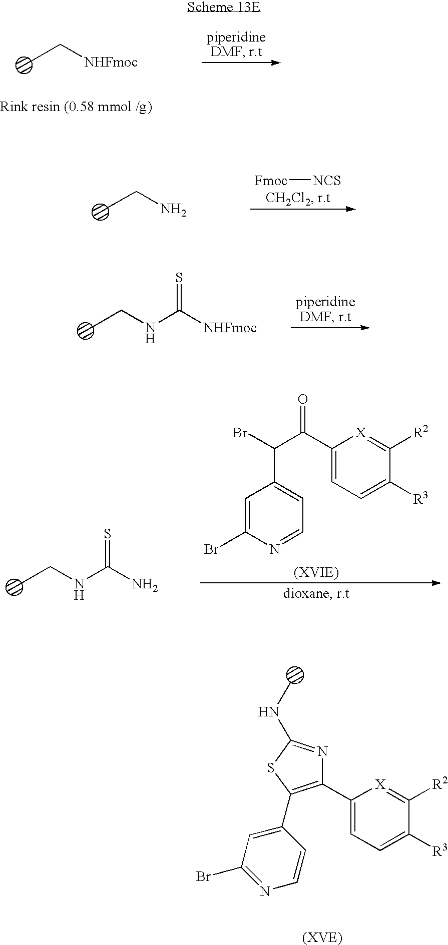

Further details for the preparation of compounds of formula (I) are found in the examples section hereinafter.

The compounds of the invention may be prepared singly or as compound libraries comprising at least 2, for example 5 to 1,000 compounds, and more preferably 10 to 100 compounds. Libraries of compounds of the invention may be prepared by a combinatorial ‘split and mix’ approach or by multiple parallel synthesis using either solution phase or solid phase chemistry, by procedures known to those skilled in the art. Thus according to a further aspect there is provided a compound library comprising at least 2 compounds of the invention.

Activation of the TGF-β1 axis and expansion of extracellular matrix are early and persistent contributors to the development and progression of chronic renal disease and vascular disease. Border W. A., et al, N. Engl. J. Med., 1994; 331(19), 1286-92. Further, TGF-β1 plays a role in the formation of fibronectin and plasminogen activator inhibitor-1, components of sclerotic deposits, through the action of smad3 phosphorylation by the TGF-β1 receptor ALK5. Zhang Y., et al, Nature, 1998; 394(6696), 909-13; Usui T., et al, Invest. Ophthalmol. Vis. Sci., 1998; 39(11), 1981-9.

Progressive fibrosis in the kidney and cardiovascular system is a major cause of suffering and death and an important contributor to the cost of health care. TGF-β1 has been implicated in many renal fibrotic disorders. Border W. A., et al, N. Engl. J. Med., 1994; 331(19), 1286-92. TGF-β1 is elevated in acute and chronic glomerulonephritis Yoshioka K., et al, Lab. Invest., 1993; 68(2), 154-63, diabetic nephropathy Yamamoto, T., et al, 1993, PNAS 90, 1814-1818, allograft rejection, HIV nephropathy and angiotensin-induced nephropathy Border W. A., et al, N. Engl. J. Med., 1994; 331(19), 1286-92. In these diseases the levels of TGF-β1 expression coincide with the production of extracellular matrix. Three lines of evidence suggest a causal relationship between TGF-β1 and the production of matrix. First, normal glomeruli, mesangial cells and non-renal cells can be induced to produce extracellular-matrix protein and inhibit protease activity by exogenous TGF-β1 in vitro. Second, neutralizing anti-bodies against TGF-β1 can prevent the accumulation of extracellular matrix in nephritic rats. Third, TGF-β1 transgenic mice or in vivo transfection of the TGF-β1 gene into normal rat kidneys resulted in the rapid development of glomerulosclerosis. Kopp J. B., et al, Lab. Invest., 1996; 74(6), 991-1003. Thus, inhibition of TGF-β1 activity is indicated as a therapeutic intervention in chronic renal disease.

TGF-β1 and its receptors are increased in injured blood vessels and are indicated in neointima formation following balloon angioplasty Saltis J., et al, Clin. Exp. Pharmacol. Physiol., 1996; 23(3), 193-200. In addition TGF-β1 is a potent stimulator of smooth muscle cell (“SMC”) migration in vitro and migration of SMC in the arterial wall is a contributing factor in the pathogenesis of atherosclerosis and restenosis. Moreover, in multivariate analysis of the endothelial cell products against total cholesterol, TGF-β receptor ALK5 correlated with total cholesterol (P<0.001) Blann A. D., et al, Atherosclerosis, 1996; 120(1-2), 221-6. Furthermore, SMC derived from human atherosclerotic lesions have an increased ALK5/TGF-β type II receptor ratio. Because TGF-β1 is over-expressed in fibroproliferative vascular lesions, receptor-variant cells would be allowed to grow in a slow, but uncontrolled fashion, while overproducing extracellular matrix components McCaffrey T. A., et al, Jr., J. Clin. Invest., 1995; 96(6), 2667-75. TGF-β1 was immunolocalized to non-foamy macrophages in atherosclerotic lesions where active matrix synthesis occurs, suggesting that non-foamy macrophages may participate in modulating matrix gene expression in atherosclerotic remodelling via a TGF-β-dependent mechanism. Therefore, inhibiting the action of TGF-β1 on ALK5 is also indicated in atherosclerosis and restenosis.

TGF-β is also indicated in wound repair. Neutralizing antibodies to TGF-β1 have been used in a number of models to illustrate that inhibition of TGF-β1 signalling is beneficial in restoring function after injury by limiting excessive scar formation during the healing process. For example, neutralizing antibodies to TGF-β1 and TGF-β2 reduced scar formation and improved the cytoarchitecture of the neodermis by reducing the number of monocytes and macrophages as well as decreasing dermal fibronectin and collagen deposition in rats Shah M., J. Cell. Sci., 1995, 108, 985-1002. Moreover, TGF-β antibodies also improve healing of corneal wounds in rabbits Moller-Pedersen T., Curr. Eye Res., 1998, 17, 736-747, and accelerate wound healing of gastric ulcers in the rat, Ernst H., Gut, 1996, 39, 172-175. These data strongly suggest that limiting the activity of TGF-β would be beneficial in many tissues and suggest that any disease with chronic elevation of TGF-β would benefit by inhibiting smad2 and smad3 signalling pathways.

TGF-β is also implicated in peritoneal adhesions Saed G. M., et al, Wound Repair Regeneration, 1999 November-December, 7(6), 504-510. Therefore, inhibitors of ALK5 would be beneficial in preventing peritoneal and sub-dermal fibrotic adhesions following surgical procedures.

TGF-β is also implicated in photoaging of the skin (see Fisher G J. Kang S W. Varani J. Bata-Csorgo Z. Wan Y S. Data S. Voorhees J J. , Mechanisms of photoaging and chronological skin ageing, Archives of Dermatology, 138(11):1462-1470, 2002 November and Schwartz E. Sapadin A N. Kligman L H. “Ultraviolet B radiation increases steady state mRNA levels for cytokines and integrins in hairless mouse skin-modulation by topical tretinoin”, Archives if Dermatological Research, 290(3):137-144, 1998 March.)

Therefore according to a further aspect, the invention provides the use of a compound defined in the first aspect in the preparation of a medicament for treating or preventing a disease or condition mediated by ALK-5 inhibition.

Preferably the disease or condition mediated by ALK-5 inhibition is selected from the list: chronic renal disease, acute renal disease, wound healing, arthritis, osteoporosis, kidney disease, congestive heart failure, ulcers (including diabetic ulcers, chronic ulcers, gastric ulcers, and duodenal ulcers), ocular disorders, corneal wounds, diabetic nephropathy, impaired neurological function, Alzheimer's disease, atherosclerosis, peritoneal and sub-dermal adhesion, any disease wherein fibrosis is a major component, including, but not limited to kidney fibrosis, lung fibrosis and liver fibrosis, for example, hepatitis B virus (HBV), hepatitis C virus (HCV), alcohol-induced hepatitis, haemochromatosis, primary biliary cirrhosis, restenosis, retroperitoneal fibrosis, mesenteric fibrosis, endometriosis, keloids, cancer, abnormal bone function, inflammatory disorders, scarring and photaging of the skin.

More preferably the disease or condition mediated by ALK-5 inhibition is fibrosis. Preferably kidney fibrosis.

It will be appreciated that references herein to treatment extend to prophylaxis as well as the treatment of established conditions.

Compounds of the invention may be administered in combination with other therapeutic agents, for example antiviral agents for liver diseases, or in combination with ACE inhibitors or angiotensin II receptor antagonists for kidney diseases.

The compounds of the invention may be administered in conventional dosage forms prepared by combining a compound of the invention with standard pharmaceutical carriers or diluents according to conventional procedures well known in the art. These procedures may involve mixing, granulating and compressing or dissolving the ingredients as appropriate to the desired preparation.

The pharmaceutical compositions of the invention may be formulated for administration by any route, and include those in a form adapted for oral, topical or parenteral administration to mammals including humans.

The compositions may be formulated for administration by any route. The compositions may be in the form of tablets, capsules, powders, granules, lozenges, creams or liquid preparations, such as oral or sterile parenteral solutions or suspensions.

The topical formulations of the present invention may be presented as, for instance, ointments, creams or lotions, eye ointments and eye or ear drops, impregnated dressings and aerosols, and may contain appropriate conventional additives such as preservatives, solvents to assist drug penetration and emollients in ointments and creams.

The formulations may also contain compatible conventional carriers, such as cream or ointment bases and ethanol or oleyl alcohol for lotions. Such carriers may be present as from about 1% up to about 98% of the formulation. More usually they will form up to about 80% of the formulation.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

Suppositories will contain conventional suppository bases, e.g. cocoa-butter or other glyceride.

For parenteral administration, fluid unit dosage forms are prepared utilising the compound and a sterile vehicle, water being preferred. The compound, depending on the vehicle and concentration used, can be either suspended or dissolved in the vehicle. In preparing solutions the compound can be dissolved in water for injection and filter sterilised before filling into a suitable vial or ampoule and sealing.

Advantageously, agents such as a local anaesthetic, preservative and buffering agents can be dissolved in the vehicle. To enhance the stability, the composition can be frozen after filling into the vial and the water removed under vacuum. The dry lyophilised powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilisation cannot be accomplished by filtration. The compound can be sterilised by exposure to ethylene oxide before suspending in the sterile vehicle. Advantageously, a surfactant or wetting agent is included in the composition to facilitate uniform distribution of the compound.

The compositions may contain from 0.1% by weight, preferably from 10-60% by weight, of the active material, depending on the method of administration. Where the compositions comprise dosage units, each unit will preferably contain from 50-500 mg of the active ingredient. The dosage as employed for adult human treatment will preferably range from 100 to 3000 mg per day, for instance 1500 mg per day depending on the route and frequency of administration. Such a dosage corresponds to 1.5 to 50 mg/kg per day. Suitably the dosage is from 5 to 20 mg/kg per day.

It will be recognised by one of skill in the art that the optimal quantity and spacing of individual dosages of a compound of the invention will be determined by the nature and extent of the condition being treated, the form, route and site of administration, and the particular mammal being treated, and that such optimums can be determined by conventional techniques. It will also be appreciated by one of skill in the art that the optimal course of treatment, i.e., the number of doses of a compound of the invention given per day for a defined number of days, can be ascertained by those skilled in the art using conventional course of treatment determination tests.

No toxicological effects are indicated when a compound of the invention is administered in the above-mentioned dosage range.

All publications, including, but not limited to, patents and patent applications cited in this specification, are herein incorporated by reference as if each individual publication were specifically and individually indicated to be incorporated by reference herein as though fully set forth.

It will be appreciated that the invention includes the following further aspects. The preferred embodiments described for the first aspect extend these further aspects:

i) a pharmaceutical composition comprising a compound of the invention and a pharmaceutically acceptable carrier or diluent;

ii) a compound of the invention for use as a medicament;

iii) a method of treatment or prophylaxis of a disorder selected from chronic renal disease, acute renal disease, wound healing, arthritis, osteoporosis, kidney disease, congestive heart failure, ulcers (including diabetic ulcers, chronic ulcers, gastric ulcers, and duodenal ulcers), ocular disorders, corneal wounds, diabetic nephropathy, impaired neurological function, Alzheimer's disease, atherosclerosis, peritoneal and sub-dermal adhesion, any disease wherein fibrosis is a major component, including, but not limited to kidney fibrosis, lung fibrosis and liver fibrosis, for example, hepatitis B virus (HBV), hepatitis C virus (HCV), alcohol-induced hepatitis, haemochromatosis, primary biliary cirrhosis, restenosis, retroperitoneal fibrosis, mesenteric fibrosis, endometriosis, keloids, cancer, abnormal bone function, inflammatory disorders, scarring and photoaging of the skin, in mammals, which comprises administration to the mammal in need of such treatment, an effective amount of a compound of the invention; and

iv) a combination of a compound of the invention with an ACE inhibitor or an angiotensin II receptor antagonist.

According to a further aspect, the invention provides a compound of formula (I), a pharmaceutically acceptable salt, solvate or derivative thereof,

wherein

X is N or CH;
A is selected from the list: furan, dioxolane, thiophene, pyrrole, imidazole, pyrrolidine, pyran, pyridine, pyrimidine, morpholine, piperidine, oxazole, isoxazole, oxazoline, oxazolidine, thiazole, isothiazole, thiadiazole, benzofuran, indole, isoindole, indazole, imidazopyridine, quinazoline, quinoline, isoquinoline and triazole;
R¹ is selected from H, C₁₋₆alkyl, C₁₋₆alkenyl, C₁₋₆alkoxy, halo, cyano, perfluoro C₁₋₆alkyl, perfluoroC₁₋₆alkoxy, —NR⁵R⁶, —(CH₂)ₙNR⁵R⁶, —O(CH₂)ₙOR⁵, —O(CH₂)ₙNR⁵R⁶, —CONR⁵R⁶, —CO(CH₂)ₙNR⁵R⁶, —SO₂R⁵, —SO₂NR⁵R⁶, —NR⁵SO₂R⁵ and —NR⁵COR⁶;
R² is selected from H, C₁₋₆alkyl, halo, CN or perfluoroC₁₋₆alkyl;
R³ is selected from H or halo;
R⁴ is selected from H, halo, C₁₋₆alkyl or —NR⁵R⁶;
R⁵ and R⁶ are independently selected from H or C₁₋₆alkyl; or R⁵R⁶ together with the atom to which they are attached form a 3, 4, 5, 6 or 7-membered saturated or unsaturated ring which may contain one or more heteroatoms selected from N, S or O, and wherein the ring may be further substituted by one or more substituents selected from halo (such as fluoro, chloro, bromo), —CN, —CF₃, —OH, —OCF₃, C₁₋₆ alkyl and C₁₋₆alkoxy; and
n is 1-4;
   with the provisos that:

a) when A is thiazole (wherein the thiazole sulfur is on the same side as the 4-pyridyl moiety); X is N; R¹ is hydrogen, C₁₋₆alkyl, C₁₋₆alkoxy, halo, cyano, perfluoroC₁₋₆alkyl or perfluoroC₁₋₆alkoxy; R² is hydrogen, C₁₋₆alkyl, halo, cyano or perfluoroC₁₋₆alkyl; and R³ is hydrogen or halo; then R⁴ is not NH₂; and

b) when X is N, A is pyrazole (where the ring containing X is attached to the pyrazole ring at carbon atom next to a pyrazole ring nitrogen), R² is hydrogen then R³ is not hydrogen.

The following examples illustrate the present invention.

Abbreviations

| | |
|---|---|
| Binap | 2,2′-Bis(diphenylphosphino)-1,1′-binaphthyl |
| CH₂Cl₂ | dichloromethane |
| Cul | copper(I) iodide |
| DCE | dichloroethane |
| DMF | dimethylformamide |
| DMF.DMA - | dimethylformamide dimethylacetal |
| DME | 1,2-Dimethoxyethane |
| DMSO | dimethylsulfoxide |
| EDCl | 1-[3-(Dimethylamino)propyl]-3-ethylcarbodiimide |
| | hydrochloride |
| EtOAc | ethyl acetate |
| Et₂O | diethyl oxide |
| EtOH | ethanol |
| Et₃N | triethylamine |
| Fmoc-NCS | fluoromethylcarbonyl isothiocyanate |
| HOBT | hydroxybenzotriazole |
| K₂CO₃ | potassium carbonate |
| KMnO₄ | potassium permanganate |
| LiAlH₄ | lithium aluminium hydride |
| MeCN | acetonitrile |
| MeOH | methanol |
| Na₂CO₃ | sodium carbonate |
| NaH | sodium hydride |
| NaHB(OAc)₃ | sodium triacetoxyborohydride |
| NaHCO₃ | sodium hydrogen carbonate |
| NaHMDS | sodium bis(trimethylsilyl)amide |
| NaNO₂ | sodium nitrite |
| NaOH | sodium hydroxide |
| NH₄Cl | ammonium chloride |
| Na₂SO₄ | sodium sulfate |
| Pd₂(dba)₃ | tris(dibenzylideneacetone)dipalladium(0) |
| Pd(PPh₃)₄ | tetrakis(triphenylphosphine)palladium (0) |
| PTS | para-toluenesulfonic acid |
| TEA | triethylamine |
| TFA | trifluoroacetic acid |
| THF | tetrahydrofuran |
| TMEDA | N,N,N′,N′-Tetramethylethylenediamine |
| TMS | trimethylsilyl |
| TMSN₃ | trimethylsilyl azide |
| t-BuOK | potassium tert-butoxide |

Intermediate 1: 2-Chloro-4-iodo-pyridine

To a solution of 4-amino-2-chloro-pyridine (8.09 g, 63 mmol, 1 eq) in water (150 mL) cooled to 0° C. was added concentrated 98% HCl. A solution of sodium nitrite (5.65 g, 82 mmol, 1.3 eq) in water (50 mL) was added slowly at −10° C. and the mixture was stirred at this temperature for 40 min. A solution of potassium iodide (12.55 g, 75.6 mmol, 1.2 eq) in water (50 mL) was added and the resulting mixture was stirred at 0° C. overnight. After treatment with NaOH (35%) and extraction with ethyl acetate, the organic phases were combined and dried over Na₂SO₄. The solvent was removed under reduced pressure and the residue was purified by chromatography on silica gel (eluent: CH₂Cl₂ then CH₂Cl₂/CH₃OH 99:1) to give the title compound as an orange solid (9.5 g, 63%); ¹H NMR (300 MHz, CDCl₃) δ ppm: 7.99 (1H, d), 7.68 (1H, s), 7.52 (1H, d); (GC-MS) m/z: 239.

Intermediate 2: 2-Methyl-6-trimethylsilanylethynyl-pyridine

To a solution of 2-bromo-4-methyl-pyridine (25 g, 0.15 mol) in dry THF (200 mL) were added TMEDA (200 mL) and TMS-acetylene (100 mL, excess) under N₂.The resulting mixture was degassed with nitrogen for 10 min and then tetrakis(triphenlyphosphine) palladium(0) (3.7 mmol, 4.3 g) and copper iodide (14.7 mmol, 2.8 g) were added and the mixture was heated at 60° C. for 18 h. The reaction mixture was concentrated and the residue partitioned between ethyl acetate/water. The organic phase was dried over Na₂SO₄ and filtered. Evaporation of the solvent in vacuo gave a crude product which was purified by chromatography on silica gel (CH₂Cl₂) to give the title compound (18.4 g, 65%) as a black oil; ¹H NMR (300 MHz, CDCl₃) δ ppm: 7.58-7.49 (m, 1H), 7.30 (d, 1H), 7.10 (d, 1H), 2.56 (s, 3H), 0.28 (s, 9H).

Intermediate 3: 2-Ethynyl-6-methyl-pyridine

To a solution of intermediate 2 (18.4 g, 0.097 mol) in MeOH (100 ml) was added potassium carbonate (4 eq, 0.39 mol, 53.7 g) and the reaction mixture was stirred at rt for 30 min. The solvent was removed and the residue was partitioned between ethyl acetate/water. The organic layer was dried over Na₂SO₄, filtered and the solvent evaporated under reduced pressure to give the title compound (8.75 g, 77%) as a brown oil; ¹H NMR (300 MHz, CDCl₃) δ ppm: 7.45-7.34 (m, 1H), 7.14 (d, 1H), 6.98 (d, 1H), 2.97 (s, 1H), 2.40 (s, 3H).
Intermediate 4: 6-Methyl-2-[(2-chloro-pyridin-4-yl)-ethynyl]-pyridine

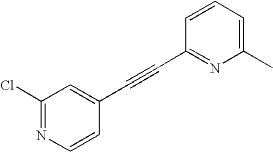

To a solution of intermediate 1 (1.85 g, 7.74 mmol) in dry THF (40 mL) were added under nitrogen TMEDA (20 mL) and intermediate 3 (1.1 eq, 1 g, 8.51 mmol). The resulting mixture was degassed with nitrogen for ten mins, tetrakis(triphenlyphosphine)palladium(0) (0.537 g, 0.464 mmol) and copper iodide (0.177 g, 0.928 mmol) were added and the mixture was heated at 60° C. for 4 hours. The mixture was poured into a saturated solution of NH₄Cl and extracted with EtOAc. The organic phase was dried over Na₂SO₄ and filtered. The solvent was removed under reduced pressure and the residue was purified by chromatography on silica gel (CH₂Cl₂/EtOAc 90:10) to afford the title compound as a beige solid (1.54 g, 86.4%); ¹H NMR (300 MHz, CDCl₃) δ ppm: 8.29 (d, 1H), 7.52 (t, 1H), 7.39 (s, 1H), 7.34-7.24 (m, 2H), 7.10 (d, 1H), 2.50 (s, 3H).
Intermediate 5: 2-[(2-(4-methylsulfonylphenyl)-pyridin-4-yl)-ethynyl]-6-methyl-pyridine

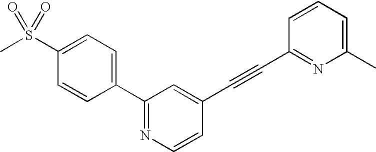

A solution of intermediate 4 (1 g, 4.37 mmol), 4-(methylsulfonyl)phenyl boronic acid (1.14 g, 5.7 mmol), tetrakis(triphenlyphosphine) palladium(0) (0.118 g, 0.1 mmol) and aqueous sodium carbonate 2M (8.6 mL, 17.2 mmol) in toluene (30 mL) and EtOH (10 mL) under nitrogen was stirred under reflux for 6 h. The mixture was hydrolysed with water, extracted with ethyl acetate and the combined organic phases were washed with water and dried over Na₂SO₄. The solvent was evaporated under reduced pressure and the crude product was purified by chromatography on silica gel (eluent:CH₂Cl₂/CH₃OH 98:2) to give the title compound as a yellow oil (0.7 g, 46%); ¹H NMR (300 MHz, CDCl₃) δ ppm: 8.66 (d, 1H), 8.14 (d, 2H), 7.98 (d, 2H), 7.90 (s, 1H), 7.56 (t, 1H), 7.43-7.32 (m, 2H), 7.12 (d, 1H), 3.03 (s, 3H), 2.50 (s, 3H); [APCl MS] m/z 349 (MH⁺).

The following compounds of formula (IIBc) were prepared by methods analogous to that described for intermediate 5 using the appropriate boronic acid derivative (see Table 1).

TABLE 1

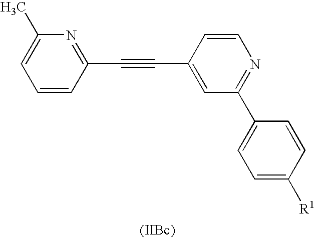

| | | From | |
|---|---|---|---|
| Int. | R¹ | intermediates: | Physical data |
| 6 | methoxy | 4 | APCI MS m/z 301 (MH+) |
| 7 | tetrahydropyran-4- | 4, 33 | APCI MS m/z 398 (MH+) |
| | ylaminocarbonyl | | |
| 8 | hydroxy | 4 | APCI MS m/z 287 (MH+) |
| 9 | ethyl | 4 | APCI MS m/z 299 (MH+) |
| 10 | chloro | 4 | APCI MS m/z 306 (MH+) |
| 11 | trifluoromethoxy | 4 | APCI MS m/z 355 (MH+) |
| 12 | 2-(pyrrolidino)ethoxy | 4, 29 | APCI MS m/z 384 (MH+) |
| 13 | fluoro | 4 | APCI MS m/z 289 (MH+) |
| 14 | formyl | 4 | APCI MS m/z 299 (MH+) |

Intermediate 15: 4-{4-[4-(6-Methyl-pyridin-2-ylethynyl)-pyridin-2-yl]-benzyl}-morpholine

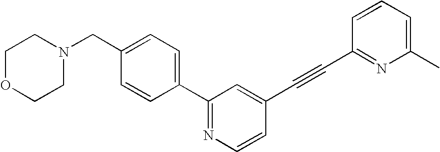

To a solution of intermediate 14 (0.45 g, 1.5 mmol) in dichloroethane (40 ml) were added morpholine (0.9 g, 6 mmol), sodium triacetoxyborohydride (1.2 g, 6 mmol) and acetic acid (0.27 g, 4 mmol). The mixture was stirred at room temperature for 6 hours and then poured into ice and extracted with CH₂Cl₂. The organic phase was dried over Na₂SO₄, filtered and concentrated under reduced pressure to give the title compound as a yellow oil (0.55 g, quantitative); [APCl MS] m/z 370 (MH+).
Intermediate 16: N,N-dimethyl-2-[(4-{4-(6-methyl-pyridin-2-yl-ethynyl)-pyridin-2-yl}phenyl)oxy]ethanamine

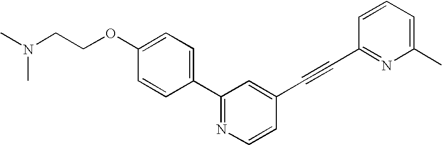

To a solution of intermediate 8 (0.572 g, 2 mmol) in acetone (20 ml) were added 2-chloro-N,N-dimethylethanamine hydrochloride (0.374 g, 2.6 mmol) and potassium carbonate (0.822 g, 6 mmol) and the mixture was stirred under reflux overnight. The reaction mixture was filtered and concentrated under reduced pressure. The residue was poured into water and extracted with CH₂Cl₂. The organic phase was dried over Na₂SO₄, filtered and concentrated under reduced pressure to give the title compound as a brown oil (0.7 g, 98%); [APCl MS] m/z=358 (MH+).

Intermediate 17: 3-chloro-4-fluoro-benzoic acid ethyl ester

To a solution of 3-chloro-4-fluoro-benzoic acid (11.75 g, 67.3 mmol) in EtOH was added PTSA (1.2 g) and the resulting mixture was heated under reflux for 2 days. On cooling the mixture was poured into water and the aqueous phase was basified with a solution of NaOH 1N. The product was extracted with CH₂Cl₂ and the organic phase was dried over Na₂SO₄ and concentrated under reduced pressure to give the title compound as an oil (13.08 g, 96%); [APCl MS] m/z 203 (MH+).

Intermediate 18: 3,4-difluoro-benzoic acid ethyl ester

3,4-Difluoro-benzoic acid (11 g, 69.57 mmol) was reacted as described for intermediate 1 to afford the title compound as an oil (11.78 g, 91%); ¹H NMR (300 MHz, CDCl₃) δ ppm: 7.84 (m, 2H), 7.22 (m, 1H), 4.37 (q, 2H), 1.38 (t, 3H).

Intermediate 19: 6-methyl-pyridine-2-carboxylic acid ethyl ester

6-Methyl-pyridine-2-carboxylic acid (25 g, 182.3 mmol) was reacted as described for intermediate 1 to afford the title compound as an oil (22.9 g, 76.13%); ¹H NMR (300 MHz, CDCl₃) δ ppm: 7.95 (d, 1H), 7.75 (t, 1H), 7.35 (d, 1H), 4.5 (q, 2H), 2.7 (s, 3H), 1.45 (t, 3H).

Intermediate 20: 6-fluoro-pyridine-2-carboxylic acid

To a solution of 2-fluoro-6-methyl-pyridine (2.5 g, 22.5 mmol) in water (170 ml) was added portionwise KMnO₄ (2 g, 12.65 mmol) and the mixture was heated to reflux. Then KMnO₄ (8 g, 50.63 mmol) was added portionwise and the mixture was heated under reflux for 3 hours. On cooling, the precipitate was filtered and the filtrate was acidified with a solution of HCl and then concentrated under reduced pressure. The residue was triturated with hot EtOH, the solid was filtered and the filtrate was concentrated to dryness under reduced pressure. The title compound was obtained as a white solid (1.7 g, 53%); m.p. 137° C.

Intermediate 21: 6-fluoro-pyridine-2-carboxylic acid isopropyl ester

Intermediate 20 (1 g, 7.09 mmol) was added portionwise to thionyl chloride (3 ml) and the mixture was heated under reflux for 3 hours and then concentrated under reduced pressure. Isopropanol (3 ml) was added to the residue and the mixture was stirred at room temperature for 5 minutes and then concentrated under reduced pressure. The residue was treated with a saturated solution of NaHCO₃, extracted with ethyl acetate, the organic phase was dried over Na₂SO₄ and concentrated under reduced pressure. The title compound was obtained as an cream oil (1.2 g, 93%); [APCl MS] m/z: 184 (MH+).

Intermediate 22: 1-methyl-4-hydroxymethyl-imidazole

To a suspension of 1-methyl-imidazole-4-carboxylic acid (11.4 g, 90 mmol) in THF (500 ml) at 0° .C was added dropwise LiAlH₄ (solution 1M in THF, 117 ml, 117 mmol) and the mixture was stirred at room temperature overnight and then at 50° C. for 1 hour. On cooling, water (3 ml) was added followed by Na₂SO₄, and the resulting precipitate was filtered through a celite pad. The filtrate was concentrated under reduced pressure to afford the title compound as a solid (8 g, 78.95%); ¹H NMR (300 MHz, CDCl₃) δ ppm: 7.25 (s, 1H), 6.7 (s, 1H), 5.25 (m, 1H), 4.4 (s, 2H), 3.45 (s, 3H).

Intermediate 23: 1-methyl-4-chloromethyl-imidazole hydrochloride

To a solution of intermediate 22 (5 g, 44.64 mmol) in CH₂Cl₂ (10 ml) at 0° C. was added dropwise thionyl chloride (50 ml) and then the mixture was stirred at room temperature overnight and then at reflux for 3 hours. The mixture was concentrated under reduced pressure, and diethyl ether added. The resulting precipitate was filtered and dried to give the title product as a brown solid (4 g, 53.81%); ¹H NMR (300 MHz, d⁶-DMSO) δ ppm: 9.25 (s, 1H), 7.8 (s, 1H), 4.95 (s, 2H), 3.9 (s, 3H).

Intermediate 24: 4-(morpholin-4-yl)-bromobenzene

To a solution of 4-phenylmorpholine (18 g, 110.4 mmol) in ethanol (400 ml) cooled in an iced bath, was added dropwise bromine (5.95 ml, 115.9 mmol) and the mixture was stirred at room temperature for 2 hours. The mixture was poured into water and the solution was made basic by addition of a solution of sodium hydroxide (1N). The resulting precipitate was filtered, washed with water and dried. Crystallisation from diisopropyl ether gave the title compound as white crystals (15 g, 56.13%); m.p. 126-128° C.

Intermediate 25: 1-ethyl-4-(4-bromophenyl)-piperazine

To a solution of 1-ethyl-4-phenylpiperazine (18 g, 95 mmol) in ethanol (600 ml) cooled in an iced bath, was added dropwise bromine (5.1 ml, 99 mmol) and the mixture was stirred at room temperature for 2 hours. The mixture was poured into water and made basic by addition of a solution of sodium hydroxide (1N) . After extraction with CH₂Cl2, the organic phase was dried over Na₂SO₄ and concentrated under reduced pressure. The residue was purified by chromatography on silica gel eluting with CH₂Cl₂/MeOH (9/1). The titled compound was obtained as a solid (21 g, 82.4%); [APCl MS] m/z=270 (MH+).
Intermediate 26: 4-(2-(pyrrolidin-1-yl)-ethoxy)-iodobenzene

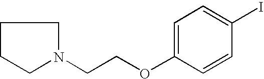

To a solution of 4-iodophenol (6 g, 27.3 mmol) in acetone (200 ml) were added caesium carbonate (22.2 g, 68.4 mmol) and N-(2-chloroethyl)-pyrrolidine hydrochloride (7 g, 41 mmol) and the mixture was heated under reflux for 4 hours. On cooling, the mixture was poured into water, and extracted with CH₂Cl₂. The organic phase was dried over Na₂SO₄ and concentrated under reduced pressure to give the title compound as a red oil (8 g, 92.53%); ¹H NMR (300 MHz, CDCl₃, ppm) δ: 7.5 (d, 2H), 6.65 (d, 2H), 4 (t, 2H), 2.8 (t, 2H), 2.55 (m, 4H), 1.75 (m, 4H).
Intermediate 27: N-[4-(4,4,5,5-tetramethyl-[1,3,2]-dioxaborolan-2-yl)-phenyl]-morpholine

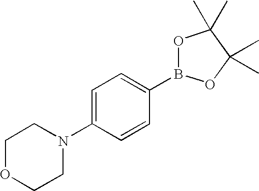

To a solution of intermediate 24 (20 g, 82.64 mmol) in dioxane (200 ml) was added 4,4,5,5-tetramethyl-[1,3,2]-dioxaborolane (13.2 ml, 99.17 mmol), dichloro bis(triphenylphosphine) palladium(II) (3 g, 4.13 mmol) and triethylamine (34.5 ml, 247.93 mmol) and the mixture was heated under reflux during 4 hours and then poured into water. After extraction with CH₂Cl₂, the organic phase was dried over Na₂SO₄ and concentrated under reduced pressure. The residue was purified by chromatography on silica gel (CH₂Cl₂) to give the title compound as an orange oil which crystallised (19.98 g, 83.94%); [APCl MS] m/z 289.07 (MH+).
Intermediate 28: 1-ethyl-4-[4-(4,4,5,5-tetramethyl-1,3,2-dioxaborolan-2-yl)phenyl]-piperazine

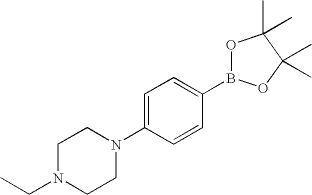

To a solution of intermediate 25 (3 g, 11 mmol) in dioxane (100 ml) was added 4,4,5,5-tetramethyl-1,3,2-dioxaborolane (1.8 ml, 12 mmol), dichlorobis(triphenylphosphine)palladium(II) (0.392 g, 0.57 mmol) and triethylamine (4.65 ml, 33 mmol) and the mixture was heated under reflux for 12 hours. On cooling, the mixture was poured into water and extracted with CH₂Cl₂. The organic phase was dried over Na₂SO₄ and concentrated under reduced pressure to give a residue which was purified by chromatography on silica gel eluting with CH₂Cl₂/MeOH (90:10). The titled compound was obtained as a brown oil which crystallised on standing (2 g, 55.48%); m.p. 130-134° C.
Intermediate 29: 1-[2-(pyrrolidin-1-yl)-ethoxy]-4-[4,4,5,5-tetramethyl-1,3,2-dioxaborolan-2-yl]-benzene

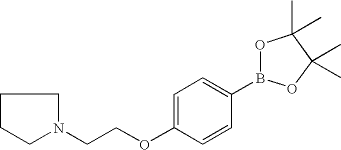

Intermediate 26 (8 g, 25.24 mmol) and 4,4,5,5-tetramethyl-1,3,2dioxaborolane (4 ml, 27.6 mmol) were reacted as described for intermediate 27 to afford the titled compound as a solid (8 g, 99.99%); m.p. 160-164° C.
Intermediate 30: 1-[aminocarbonylmethyloxy]-4-[4,4,5,5-tetramethyl-1,3,2-dioxaborolan-2-yl]-benzene

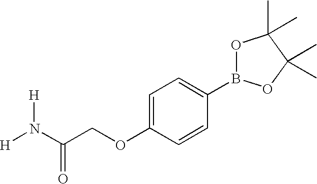

To a solution of 4-[4,4,5,5-tetramethyl-1,3,2-dioxaborolan-2-yl]-phenol (5 g, 22.7 mmol) in acetone (80 ml) were added cesium carbonate (10.37 g, 32 mmol) and bromoacetamide (4.39 g, 32 mmol) and the mixture was heated at 70° C. for 3 hours. On cooling, the mixture was concentrated under reduced pressure and the residue was treated with water and extracted with CH₂Cl₂. The organic phase was dried over Na₂SO₄, and concentrated. After trituration with diisopropyl ether, the title compound was obtained as a solid (4 g, 63.54%); m.p. 166-168° C.
Intermediate 31: 1-[(1-methyl-imidazol-4-yl)-methyloxy]-4-[4,4,5,5-tetramethyl-1,3,2-dioxaborolan-2-yl]-benzene

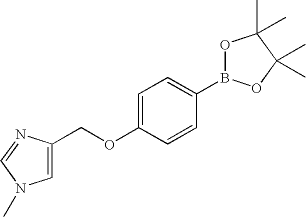

4-[4,4,5,5-Tetramethyl-1,3,2-dioxaborolan-2-yl]phenol (1 g, 4.54 mmol) and intermediate 22 (1.88 g, 11.4 mmol) were reacted as described for intermediate 29, to afford, after chromatography on silica gel (CH₂Cl₂/MeOH, 95/5), the title compound as a pale yellow oil (0.5 g, 35%); ¹H NMR (300 MHz, CDCl₃, ppm) δ:7.6 (d, 2H), 7.3 (s, 1H), 6.8 (m, 3H), 4.9 (s, 2H), 3.5 (s, 3H), 1.2 (s, 12H).
Intermediate 32: 1-[(morpholin-4-yl)carbonylmethyloxy]-4-[4,4,5,5-tetramethyl-1,3,2-dioxaborolan-2-yl]-benzene

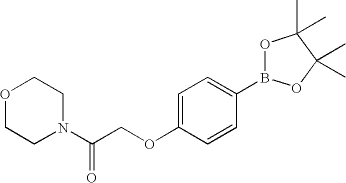

To a solution of 4-[4,4,5,5-tetramethyl-1,3,2-dioxaborolan-2-yl]-phenol (6.6 g, 30 mmol) in CH₃CN were added potassium carbonate (12.42 g, 90 mmol) and N-(chloroacetyl)-morpholine (4.89 g, 30 mmol) and the mixture was heated under reflux for 3 hours. On cooling the mixture was concentrated under reduced pressure, and the residue was treated with water and extracted with EtOAc. The organic phase was dried over Na₂SO₄ and concentrated. Trituration from hexane gave the title compound as a grey solid (9.5 g, 91%); m.p. 112° C.; [APCl MS] m/z 348 (MH+).
Intermediate 33: 4-(4,4,5,5-tetramethyl-[1,3,2]dioxaborolan-2-yl)-N-(tetrahydro-pyran-4-yl)-benzamide

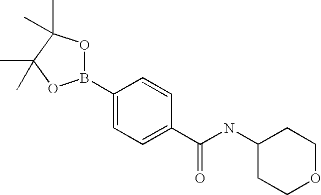

4-(4,4,5,5-tetramethyl-[1,3,2]dioxaborolan-2-yl)-benzoic acid (70.16 g, 0.28 mol) was treated with SOCl₂ (2 vol.) and the reaction mixture was stirred to reflux for 2 hours. After evaporation, the residue was diluted in toluene and poured into a solution at 10° C. of tetrahydro-pyran-4-ylamine (34.34 g, 0.339) and triethylamine (79 mL, 0.57 mol) in CH₂Cl₂. The reaction mixture was stirred at room temperature during 2 days and water (490 mL) was added to give a precipitate which was filtered off and washed with EtOAc. After purification by flash chromatography using CH₂Cl₂/MeOH (95:5). The title compound was obtained as a solid (17.02 g, 18%); ¹H NMR (400 MHz, CDCl₃, ppm) δ: 7.85 (d, 2H), 7.72 (d, 2H), 5.98 (m, 1H), 4.20 (s, 1H), 3.99 (m, 2H), 3.35 (t, 2H), 2.01 (d, 2H), 1.57 (m, 2H), 1.35 (s, 12H).
Intermediate 34: N-[4-(4,4,5,5-tetramethyl-[1,3,2]dioxaborolan-2-yl)-phenyl]-methanesulfonamide

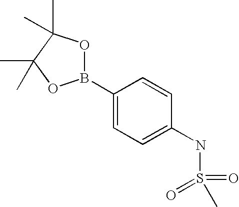

To a solution of 4-(4,4,5,5-tetramethyl-[1,3,2]dioxaborolan-2-yl)-aniline (Aldrich, 5 g, 22.8 mmol) in CH₂Cl₂ (20 ml) was added NaHCO₃ (2.3 g, 27.4 mmol) and methanesulfonyl chloride (13.2 mL, 171 mmol) and the reaction mixture was stirred at room temperature during 6 days. Water was added and the product was extracted with CH₂Cl₂. The organic layer was dried over Na₂SO₄, and concentrated under reduced pressure. Crystallisation from diethyl ether gave the title compound as a white powder (2.52 g, 37%); ¹H NMR (300 MHz, CDCl₃, ppm) δ: 7.78 (d, 2H), 7.18 (d, 2H), 6.69 (m, 1H), 3.02 (s, 3H), 1.33 (s, 12H).
Intermediate 35: N-[(4(4,4,5,5-tetramethyl-[1,3,2]dioxaborolan-2-yl)-phenyl)carbonyl]-morpholine

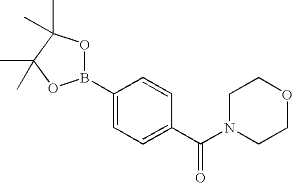

To a solution of 4-(4,4,5,5-tetramethyl-[1,3,2]dioxaborolan-2-yl)-benzoic acid (5 g, 20.15 mmol) in CH₂Cl₂/DMF (50 ml/5 ml) were added morpholine (2.1 ml, 24.2 mmol), HOBT (3.3 g, 24.2 mmol), EDCl (4.65 g, 24.2 mmol) and triethylamine (4.2 ml, 30.2 mmol) and the reaction mixture was stirred at room temperature during 3 days. Water was added and the product was extracted with CH₂Cl₂. The organic phase was dried over Na₂SO₄ and concentrated under reduced pressure. Trituration with diisopropyl ether gave the title compound as a white solid (4.21 g, 66%); ¹H NMR (300 MHz, CDCl₃, ppm) δ: 7.8 (d, 2H), 7.4 (d, 2H), 3.7 (m, 4H), 3.55 (m, 2H), 3.35 (m, 2H), 1.3 (s, 12H).
Intermediate 36: 1-ethyl-4-[(4-(4,4,5,5-tetramethyl-[1,3,2]dioxaborolan-2-yl)-phenyl)carbonyl]-piperazine

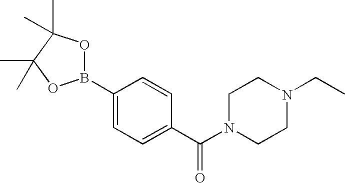

4-(4,4,5,5-Tetramethyl-[1,3,2]dioxaborolan-2-yl)-benzoic acid (8.24 g, 33.22 mmol) and N-ethylpiperazine (5.1 ml, 39.87 mmol) were reacted as described for intermediate 34 to afford, after chromatography on silica gel (CH₂Cl₂/MeOH, 95/5), the title compound as a pale yellow oil which crystallised (9.64 g, 84%); [APCl MS] m/z 345 (MH+).

Intermediate 37: 2-(4-bromophenyl)-4-methyl-pyridine

2-Bromo-4-methylpyridine (10 g, 58.14 mmol) was dissolved in toluene (100 ml) and tetrakis(triphenylphosphine)palladium(0) (5 mol %, 3.36 g) added under N₂ and degassed. Aqueous sodium carbonate (2M, 2 eq) was added slowly and stirred for 10 min. A solution of 4-bromophenylboronic acid (Lancaster, 14 g, 1.2 eq) in ethanol (20 ml) was added dropwise and the mixture was heated under reflux overnight and then poured into water. After extraction with CH₂Cl₂, the organic phase was dried over Na₂SO₄, and concentrated under reduced pressure. The resulting residue was purified by chromatography on silica gel (CH₂Cl₂/cyclohexane 6/4 then 8/2 then CH₂Cl₂). After crystallisation from pentane, the titled compound was obtained as white crystals (6.3 g, 43.7%); ¹H NMR (300 MHz, CDCl₃, ppm) δ 8.5 (d, 1H), 7.83 (d, 2H), 7.56 (d, 2H), 7.5 (s, 1H), 7.05 (m, 1H), 2.4 (s, 3H).
Intermediate 38: 2-[4-(morpholin-4-yl)phenyl]-4-methyl-pyridine

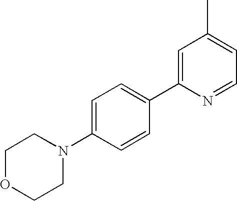

To a solution of intermediate 37 (2.66 g, 10.72 mmol) in toluene (50 ml) was added morpholine (1.12 ml, 1.2 eq, 12.9 mmol), Pd₂(dba₂)₃ (0.49 g, 0.05 eq, 0.53 mmol), binap (1 g, 0.15 eq, 1.6 mmol) and potassium tert-butoxide (1.44 g, 1.4 eq, 15 mmol) and the mixture was heated under reflux for 2 h and then poured into water. After extraction with CH₂Cl₂, the organic phase was dried over Na₂SO₄, and concentrated under reduced pressure. The resulting residue was purified by chromatography on silica gel (CH₂Cl₂/MeOH gradient from 99:1 to 95:5). The title compound was obtained as a yellow solid (2.6 g, 95.43%); ¹H NMR (300 MHz, CDCl₃, ppm) δ: 8.5 (d, 1H), 7.95 (d, 2H), 7.5 (s, 1H), 7 (m, 3H), 3.9 (m, 4H), 3.3 (m, 4H), 2.4 (s, 3H).
Intermediate 39: 2-[2-bromo-pyridin-4-yl]-1-pyridin-2-yl-ethanone

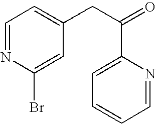

To a solution of 2-bromo-4methyl-pyridine (27 g, 157 mmol) in dry THF (270 ml) was added ethyl picolinate (28.5 g, 188.7 mmol). The resulting mixture was cooled to −78° C. under argon and a solution of sodium bis(trimethylsilyl)amide 1M in THF (345 ml, 345 mmol) was added dropwise at −78° C. The resulting reaction mixture was allowed to reach room temperature and subsequently stirred overnight. The solvent was evaporated under reduced pressure and the solid residue triturated with diethyl ether, filtered and washed with diethyl ether. The solid was then diluted with saturated NH₄Cl solution and the aqueous phase extracted with EtOAc. The organic layer was dried over sodium sulfate and concentrated. The resulting orange powder was washed with pentane to give the title compound as a yellow solid (33.97 g); m.p. 111.2° C.
Intermediate 40: 2-[2-bromo-pyridin-4-yl]-1-(6-methyl-pyridin-2-yl)-ethanone

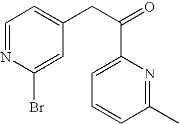

To a solution of 2-bromo-4-methyl-pyridine (5 g, 29 mmol) in dry THF (70 ml), a solution of sodium bis-(trimethylsilyl)amide 2M in THF (32 ml, 64 mmol) was added dropwise at −30° C. under nitrogen. The mixture was stirred at −30° C. for 1 h, then intermediate 19 (4.82 g, 32.3 mmol, 1.1 eq) was added. The reaction mixture was stirred at room temperature overnight. Diethyl ether was added and the precipitated solid was filtered and washed with diethyl ether. The solid was then diluted with saturated NH₄Cl solution and the aqueous phase was extracted with ethyl acetate. The organic layer was dried over Na₂SO₄ and concentrated. The resulting orange powder was washed with pentane to give the title compound as a yellow solid (5.84 g, 70%). [APCl MS] m/z 292 (MH+).
Intermediate 41: 2-(2-bromo-pyridin-4-yl)-1-(3-chloro-4-fluoro-phenyl)-ethanone

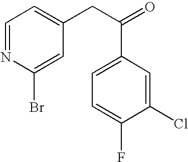

2-Bromo-4-methyl-pyridine (9.2 g, 53.5 mmol) and intermediate 17 (13 g, 64.2 mmol) were reacted as described for intermediate 39 to afford the title compound as an orange solid (17.16 g, 98%); [APCl MS] m/z: 330 (MH+).
Intermediate 42: 2-(2-bromo-pyridin-4-yl)-1-(3,4-difluoro-phenyl)-ethanone

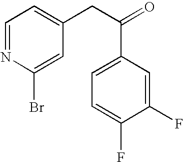

2-Bromo-4-methyl-pyridine (9.056 g, 52.64 mmol) and intermediate 18 (11.75 g, 63.17 mmol) were reacted as described for intermediate 39 to afford the title compound as an ocre solid (14.54 g, 88.5%); [APCl MS] m/z: 314 (MH+).
Intermediate 43: 2-(2-bromo-pyridin-4-yl)-1-(3-chloro-phenyl)-ethanone

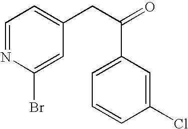

2-Bromo-4-methyl-pyridine (7.75 g , 45.1 mmol) and methyl-3-chlorobenzoate (10 g, 58.6 mmol) were reacted as described for intermediate 39 to afford the title compound as an orange powder (13.02 g, 93%); ¹H NMR (300 MHz, CDCl₃, ppm) δ: 8.34 (d, ₁H), 7.95 (m, 1H), 7.84 (d, 1H), 7.59 (d, 1H), 7.46 (d, 1H), 7.41 (d, 1H), 7.13 (d, 1H), 4.24 (s, 2H).
Intermediate 44: 2-(2-bromo-pyridin-4-yl)-1-(6-fluoro-pyridin-2-yl)-ethanone

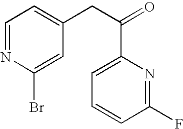

To a solution of 2-bromo-4-methyl-pyridine (2.58 g, 15 mmol) in anhydrous THF (50 ml) at −30° C., was added dropwise NaHMDS (solution 2M in THF, 15 ml, 30 mmol) and the mixture was stirred at −30° C. for 2 hours. A solution of intermediate 20 (2.74 g, 15 mmol) in THF (50 ml) was added dropwise and the mixture was stirred at −30° C. for 1 hour and then poured into water. After extraction with EtOAc, the organic phase was dried over Na₂SO₄ and concentrated under reduced pressure. The residue was purified by chromatography on silica gel (CH₂Cl₂/MeOH, 99:1). The title compound was obtained as a yellow solid (1.6 g, 36%); [APCl MS] m/z=295 (MH⁺).
Intermediate 45: 2-[2-(4-(morpholin-4-yl)phenyl)-pyridin-4-yl]-1-pyridin-2-yl-ethanone

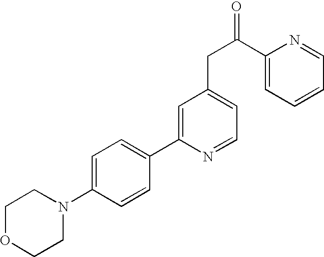

To a solution of Intermediate 38 (2.6 g, 10.24 mmol ) in dry THF (100 ml) under argon, was added dropwise a solution of sodium bis-(trimethylsilyl)amide 1M in THF (22.52 ml, 2.2 eq, 22.53 mmol). The solution was stirred room temperature for 0.5 h, then a solution of ethyl picolinate (1.66 ml, 1.2 eq, 12.3 mmol) in dry THF (20 ml) was added dropwise and the reaction mixture stirred at room temperature for 4 h. The solvent was evaporated under reduced pressure and the solid precipitated with diisopropyl ether. The brown solid was then taken up in saturated NH₄Cl solution and the aqueous phase extracted with CH₂Cl₂. The organic layer was dried over sodium sulfate and concentrated under reduced pressure to leave a residue which was purified by chromatography on silica gel (CH₂Cl₂ then CH₂Cl₂/MeOH gradient from 99:1 to 97:3). The title compound was obtained as an orange oil (1.42 g, 38.64%); ¹H NMR (300 MHz, CDCl₃, ppm) δ: 8.7 (d, 1H), 8.55 (d, 1H), 8.05 (d, 1H), 7.9 (d, 2H), 7.8 (m, 1H), 7.5 (m, 1H), 7.15 (m, 1H), 6.95 (m, 3H), 4.55 (s, 2H), 3.85 (m, 4H), 3.2 (m, 4H).
Intermediate 46: 2-[2-(4-(methanesulfonyl)phenyl)-Pyridin-4-yl]-1-(6-methylpyridin-2-yl)-ethanone

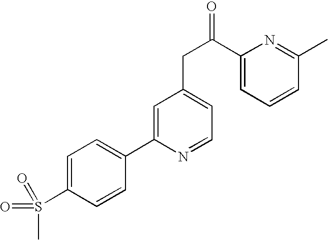

To a solution of intermediate 40 (2 g, 6.87 mmol) in DME (80 ml) was added 4-(methanesulfonyl)-phenyl boronic acid (2.1 g, 10.31 mmol), tetrakis(triphenylphosphine) palladium(0) (0.4 g, 0.35 mmol) and Na₂CO₃ (solution 2M, 22 ml) and the mixture was heated under reflux overnight and then poured into water. After extraction with CH₂Cl₂, the organic phase was dried over Na₂SO₄, and concentrated under reduced pressure. The residue was purified by chromatography on silica gel eluting with CH₂Cl₂/MeOH (95:5). The title compound was obtained as a yellow oil (1.1 g, 43.73%); [APCl MS] m/z=367 (MH⁺).

The following compounds of formula (IIEa) were prepared by methods analogous to that described for intermediate 46 using the appropriate boronic acid derivative (see Table 2).

TABLE 2

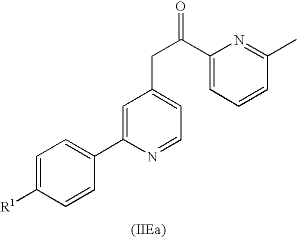

| Int. | R¹ | Physical data |
|---|---|---|
| 47 | 4-ethylpiperazin-1-yl | APCI MS m/z 387 (MH+) |
| 48 | morpholin-4-yl | APCI MS m/z 374 (MH+) |
| 49 | methoxycarbonyl | APCI MS m/z 347 (MH+) |
| 50 | formyl | APCI MS m/z 317 (MH+) |
| 51 | methoxy | APCI MS m/z 319 (MH+) |
| 52 | trifluoromethoxy | m.p. 76-78° C. |
| 53 | 2-(pyrolidin-1-yl)ethoxy | APCI MS m/z 402 (MH+) |
| 54 | aminocarbonylmethoxy | m.p. 144-146° C. |
| 55 | (1-methyl-imidazol-4-yl)methoxy | APCI MS m/z 399 (MH+) |

Intermediate 56: 2-[2-(4(carboxy)-phenyl)-pyridin-4-yl]-1-(6-methylpyridin-2-yl)-ethanone

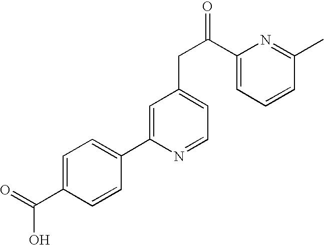

To a solution of intermediate 49 (1.2 g, 3.47 mmol) in MeOH (100 ml) was added sodium hydroxide (solution 1N, 5 ml, 5.2 mmol) and the mixture was heated under reflux for 48 hours. After cooling, a solution of HCl 1N (5 ml) was added and the precipitate was filtered and dried. The title compound was obtained as an orange solid (0.8 g, 69.5 %); [APCl MS] m/z=333 (MH⁺).
Intermediate 57: 2-[2-(4-((morpholin-4-yl)carbonyl)-phenyl)-pyridin-4-yl-1-(6-methylpyridin-2-yl)-ethanone

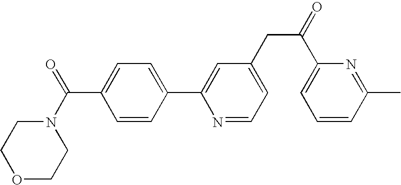

To a solution of intermediate 56 (0.8 g, 2.41 mmol) in CH₂Cl₂ (50 ml) were added morpholine (0.32 ml, 3.61mmol), HOBT (0.49 g, 3.61 mmol), EDCl (0.63 g, 3.61 mmol), triethylamine (0.84 ml, 6 mmol) and the mixture was stirred at room temperature for 24 hours and then poured into water. After extraction with CH₂Cl₂, the organic phase was dried over Na₂SO₄, and concentrated under reduced pressure. The residue was purified by chromatography on silica gel eluting with CH₂Cl₂/MeOH (9:1). The title compound was obtained as an orange oil (0.8 g, 85.35%); [APCl MS] m/z=390 (MH⁺).
Intermediate 58: 2-{2-[4-((tetrahydropyran-4-yl)-aminocarbonyl)-phenylpyridin-4-yl}-1-(6-methylpyridin-2-yl)-ethanone

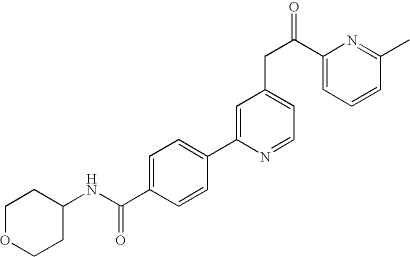

Intermediate 56 (1 g, 3 mmol) and 4-amino-tetrahydropyran (340 mg, 3.3 mmol) were reacted as described for intermediate 56 to afford, after chromatography on silica gel (CH₂Cl₂/MeOH, 9/1), the title compound as a yellow oil (0.3 g, 24%); ¹H NMR (300 MHz, CDCl₃, ppm) δ: 8.58 (d, 1H), 7.98 (m, 2H), 7.8 (m, 3H), 7.66 (m, 2H), 7.29 (m, 1H), 7.19 (m, 1H), 6.01 (m, 1H), 4.57 (s, 2H), 4.15 (m, 1H), 3.93 (m, 2H), 3.48 (m, 2H), 2.6 (s, 3H), 1.95 (m, 2H), 1.55 (m, 2H).
Intermediate 59: 2-[2-(4-((morpholin-4-yl)methyl)-phenyl)-pyridin4-yl]-1-(6-methylpyridin-2-yl)-ethanone

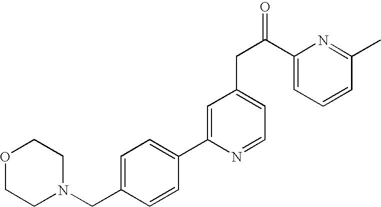

To a solution of intermediate 50 (1 g, 3.2 mmol) in CH₂Cl₂ (100 ml) were added morpholine (0.36 g, 4.1 mmol) and sodium triacetoxyborohydride (0.88 g, 4.1 mmol) and the mixture was stirred at room temperature for 3 hours and then poured into a saturated solution of NaHCO₃. After extraction with CH₂Cl₂, the organic phase was dried over Na₂SO₄ and concentrated under reduced pressure. The titled compound was obtained as a yellow oil (1.1 g, 89.82%); [APCl MS] m/z=388 (MH⁺).
Intermediate 60: [(2-(4-ethanesulfonylphenyl)-pyridin-yl)-(phenylamino)-methyl]-phosphonic acid diphenylester

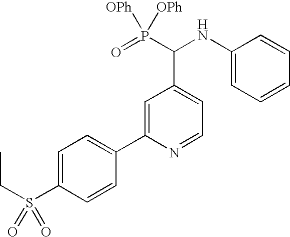

To a solution of 2-chloro-pyridine-4-carboxaldehyde (1 g, 7.06 mmol) in DME (50 ml) was added 4-(ethanesulfonyl)phenyl boronic acid (1.97 g, 9.18 mmol), tetrakis(triphenylphosphine) palladium(0) (0.816 g, 0.7 mmol) and Na₂CO₃ (solution 2M, 7 ml) and the mixture was heated under reflux overnight and then poured into water. After extraction with CH₂Cl₂, the organic phase was dried over Na₂SO₄, and concentrated under reduced pressure. The residue was purified by chromatography on silica gel eluting with CH₂CL₂/MeOH (99/1) to afford 2-(4-ethanesulfonylphenyl)-pyridine-4-carboxaldehyde as a yellow oil (1.94 g, 98%). To a solution of 2-(4-ethanesulfonylphenyl)-pyridine-4-carboxaldehyde (1.94 g, 7.06 mmol) in iPrOH were added aniline (0.772 ml, 8.47 mmol) and diphenylphosphite (1.91 ml, 9.9 mmol) and the mixture was stirred at room temperature for 18 hours and then concentrated under reduced pressure. The residue was treated with water and extracted with CH₂Cl₂, the organic phase was dried over Na₂SO₄ and concentrated. After chromatography on silica gel (CH₂Cl₂), the title compound was obtained as a yellow oil (1.45 g, 35.13%); [APCl MS] m/z 585 (MH⁺).
Intermediate 61: 1-[2-(4-ethanesulfonylphenyl)-pyridin-4-yl]-2-[6-methyl-pyridin-2-yl]-ethanone

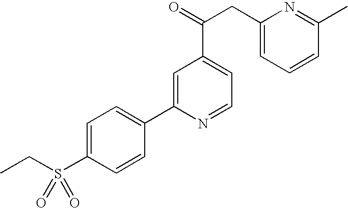

To a solution of intermediate 60 (1.45 g, 2.48 mmol) in THF/iPrOH were added 6-methyl-pyridine-2-carboxaldehyde (0.251 g, 2.07 mmol) and cesium carbonate (1.35 g, 4.14 mmol) and the mixture was stirred at room temperature for 18 hours and then neutralised with a solution of NaHCO₃. After concentration under reduced pressure, the residue was treated with water and extracted with CH₂Cl₂. The organic phase was dried over Na₂SO₄ and concentrated under reduced pressure. After chromatography on silica gel (CH₂Cl₂/MeOH, 99:1), the title compound was obtained as a yellow oil (0.321 g, 34.02%); [APCl MS] m/z 381 (MH⁺).
Intermediate 62: 2-bromo-N-methoxy-N-methyl-4-pyridinecarboxamide

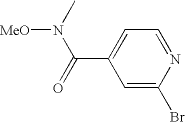

To a suspension of 2-bromo-4pyridinecarboxylic acid (23.5g, 116 mmol) in CH₂Cl₂ (600 mL) were added under nitrogen HOBT (17.3 g, 128 mmol), EDCl (24.5 g, 128 mmol), triethylamine (46.85 g, 464 mmol) and N,O-dimethylhydroxylamine hydrochloride (17.02 g, 175 mmol). The reaction mixture was stirred at room temperature for 3 hours and then partitioned between water and CH₂Cl₂. The organic phase was dried over Na₂SO₄, filtered and evaporated under reduced pressure to afford the title compound as a white solid (17 g, 59.64%); [APCl MS] m/z 246 (MH⁺).
Intermediate 63: 1-[2-bromo-pyridin-4-yl]-2-pyridin-2-yl]-ethanone

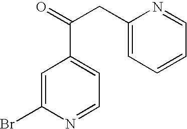

To a solution of 6-methylpyridine (2.79 g; 30 mmol) in dry THF (20 ml) under nitrogen cooled at −80° C., was added dropwise NaHMDS (solution 1M/THF, 36 ml, 36 mmol). and the mixture was stirred for 1 hour at −80° C. A solution of intermediate 62 (7.35 g; 30 mmol) in dry THF (10 mL) was added dropwise and the mixture was then stirred at room temperature overnight and then concentrated under reduced pressure. The residue was treated with hexane and the resulting precipitate was filtered. The solid was then diluted with saturated NH₄Cl solution and the aqueous phase extracted with EtOAc. The organic layer was dried over sodium sulfate and concentrated. After chromatography on silica gel (CH₂Cl₂/MeOH, 98:2), the title compound was obtained as a yellow solid (4.1 g, 49.34%); m.p. 96° C.
Intermediate 63: 1-[2-bromo-pyridin-4-yl]-2-[6methyl-pyridin-2-yl-ethanone

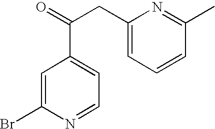

To a solution of 2,6-lutidine (4.28 g; 40 mmol) was dissolved in dry THF (100 mL) under nitrogen and the solution was cooled to −30° C. 2.5M n-Butyllithium in hexanes (16 mL; 40 mmol) was added at −30° C., then the mixture was stirred 1.5 h at ambient temperature before being cooled to −30° C to −40° C. A solution of intermediate 62 (4.9 g; 20 mmol) in dry THF (20 mL) was added at −40° C. and the reaction stirred for 2 h. Saturated aqueous ammonium chloride was added and the mixture was extracted with EtOAc. The organic phase was dried over Na₂SO₄, filtered and evaporated under reduced pressure . The residue was purified by chromatography on silica gel 60 (CH₂Cl₂/MeOH, 99/1) to give the title compound (3.42 g; 58%) as a yellow solid; m.p. 126° C.; [MS APCl] m/z: 292 (MH⁺).

The following compounds of formula (IIIEa) were prepared by methods analogous to that described for intermediate 46 using the starting materials indicated (see Table 3).

TABLE 3

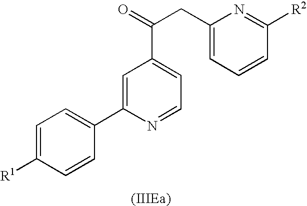

| | | | From | |
|---|---|---|---|---|
| Int. | R¹ | R² | Int. | Physical data |
| 65 | (tetrahydopyran-4- | H | 63 + 33 | APCI MS m/z 402 (MH+) |
| | yl)aminocarbonyl | | | |
| 66 | morpholln-4-yl | H | 63 + 27 | APCI MS m/z 360 (MH+) |
| 67 | chloro | CH₃ | 64 | APCI MS m/z 323 (MH+) |
| 68 | trifluoromethoxy | CH₃ | 64 | APCI MS m/z 373 (MH+) |
| 69 | (morpholin-4-yl) | CH₃ | 64 + 35 | APCI MS m/z 402 (MH+) |
| | carbonyl | | | |
| 70 | (4-ethylpiperazin-1-yl) | CH3 | 64 + 36 | APCI MS m/z 429 (MH+) |
| | carbonyl | | | |
| 71 | (tetrahydopyran-4- | CH₃ | 64 + 33 | APCI MS m/z 416 (MH+) |
| | yl)aminocarbonyl | | | |
| 72 | morpholin-4-yl | CH₃ | 64 + 27 | APCI MS m/z 374 (MH+) |
| 73 | 2-(pyrolidin-1-yl) | CH₃ | 64 + 29 | APCI MS m/z 402 (MH+) |
| | ethoxy | | | |
| 74 | aminocarbonyl- | CH₃ | 64 + 30 | APCI MS m/z 362 (MH+) |
| | methoxy | | | |
| 75 | (morpholin-4- | CH₃ | 64 + 32 | APCI MS m/z 432 (MH+) |
| | yl)carbonylmethoxy | | | |
| 76 | formyl | CH₃ | 64 | APCI MS m/z 317 (MH+) |

Intermediate 77: 2-(6-methylpyridin-2-yl)-1-[2-(4-((morpholin-4-yl)methyl)-phenyl)-pyridin-4-yl]-ethanone

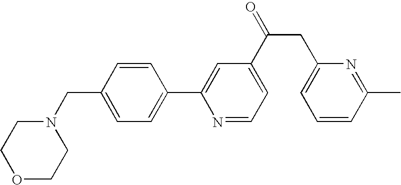

To a solution of intermediate 76 (0.984 g, 3 mmol) in 1,2-dichloroethane (40 ml) were added morpholine (0.34 g, 3.9 mmol), sodium triacetoxyborohydride (0.826 g, 3.9 mmol) and acetic acid (0.216 g, 3.6 mmol) and the mixture was stirred at room temperature for 3 hours and then poured into water. After extraction with CH₂Cl₂, the organic phase was dried over Na₂SO₄ and concentrated under reduced pressure. The title compound was obtained as an oil (1.1 g, 91%); [APCl MS] m/z 388 (MH⁺).
Intermediate 78: 2-(6-methylpyridin-2-yl)-1-[2-(4-((pyrolidin-1-yl)methyl)-phenyl)-pyridin-4-yl-ethanone

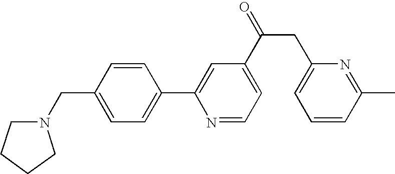

Intermediate 76 (0.7 g, 2.2 mmol) and pyrolidine (0.203 g, 2.8 mmol) were reacted as described for intermediate 76, to afford after chromatography on silica gel (CH₂Cl₂/MeOH, 9:1), the title compound as a yellow gum (0.5 g, 60.84%); [APCl MS] m/z 372 (MH⁺).
Intermediate 79: 2-(6-methylpyridin-2-yl)-1- [2-(4-((dimethylamino)methyl)-phenyl)-pyridin-4-yl]-ethanone

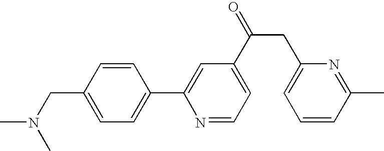

Intermediate 76 (0.7 g, 2.2 mmol) and dimethylamine (solution 2M in THF, 1.4 ml, 2.86 mmol) were reacted as described for intermediate 76, to afford after chromatography on silica gel (CH₂Cl₂/MeOH, 9:1), the title compound as a yellow gum (0.4 g, 52.34%); [APCl MS] m/z 346 (MH⁺).
Intermediate 80: 2-Bromo-4-(3-(6-methyl-pyridin-2-yl)-1H-pyrazol-4-yl)pyridine

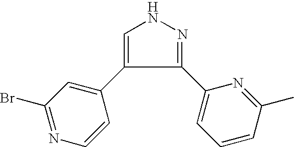

A solution of intermediate 40 (5.84 g, 20 mmol) in dry DMF (20 ml) under nitrogen was treated with glacial acetic acid (2.4 eq, 2.76 ml) over 2 min. DMF.DMA (1.5 eq., 4 ml) was added dropwise and the mixture stirred at room temperature under nitrogen for 1 hour. Hydrazine monohydrate (7.5 eq, 91 ml, 1.876 mol) was added dropwise at room temperature and the resulting mixture heated at 50° C. for 3 hours. The reaction mixture was poured into water (300 ml) and extracted with CH₂Cl₂. The organic phases were combined, dried over Na₂SO₄ and filtered. The solvent was evaporated under reduced pressure to afford a brown oil which after purification by chromatography on silica gel (eluent: CH₂Cl₂/CH₃OH 98:2) gave the title compound as a yellow solid (3.07 g, 49%); [APCl MS] m/z 315 (MH+).
Intermediate 81: 2-bromo-4-[5-methyl-3-(6-methylpyridin-2-yl)-1H-pyrazol-4-yl]pyridine

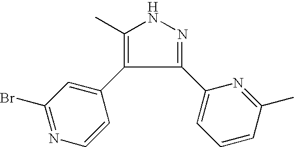

Intermediate 40 (2 g, 6.9 mmol) was reacted with N,N-dimethylacetamide dimethylacetal (1.38 g, 10 mmol) as described for intermediate 80 to afford the title compound as a brown solid (0.9 g, %); [APCl MS] m/z 328 (MH⁻).
Intermediate 82: 2-bromo-4-[3-(6-methylpyridin-2-yl)-1-trityl-1H-pyrazol-4-yl]pyridine

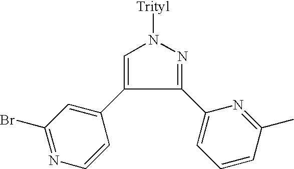

Intermediate 80 (3.07 g, 9.8 mmol) and trityl chloride (1.5 eq, 4.1 g, 14.7 mmol) were reacted with potassium carbonate (3 eq, 29.4 mmol) in acetone (100 ml). The reaction mixture was subsequently heated to reflux and stirred for 24 hours. The reaction mixture was filtered, the filtrate concentrated and then partitioned between CH₂Cl₂ and H₂O. The organic phase was dried over Na₂SO₄ and concentrated. The resulting crude material was purified by flash chromatography on silica gel, eluting with CH₂Cl₂/MeOH (98:2) to give the title compound as the major isomer of a mixture of the two isomers, as a light yellow solid (4.9 g, 90%); [APCl MS] m/z: 558 (MH⁺).
Intermediate 83: 2-bromo-4-[5-methyl-3-(6-methylpyridin-2-yl)-1-trityl-1H-pyrazol-4-yl]pyridine

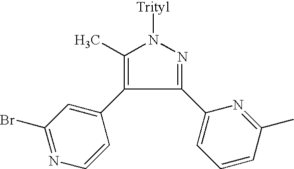

Intermediate 81 (0.9 g , 2.74 mmol) and trityl chloride (0.84 g, 3 mmol) were reacted as described for intermediate 82 to afford the title compound as a mixture of the two isomers, as a white powder (1.5 g, 95.87%); [APCl MS] m/z 329 (MH⁺ loss of trityl).
Intermediate 84: 3-(2-bromo-pyridin-4-yl)-2-(pyridin-2-yl)-imidazo[1,2-a]pyridine

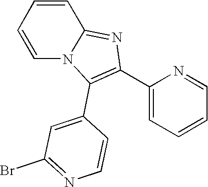

To a solution of intermediate 39 (5 g, 18.05 mmol) in CH₂Cl₂ (30 ml) was added bromine-polymer-supported (11.28 g, 18.05 mmol) and the suspension was stirred at room temperature for 5 hours. The resin was removed by filtration, with the filtrate being added directly to 2-amino-pyridine (3.4 g , 36.06 mmol) and the resin washed many times with ethanol. The filtrate was heated under reflux for 18 hours, allowed to cool and then concentrated. The residue was treated with water and extracted with CH₂Cl₂. The organic phase was dried over Na₂SO₄ and evaporated under reduced pressure to give a crude solid which was precipitated from diisopropyl ether to afford the title compound as a brown powder (3.05 g; 48%); m.p. 227° C.

The following intermediates (see Table 4) were prepared by methods analogous to that described for intermediate 84 from the starting materials indicated.

TABLE 4

| Int. | structures | From int. | Physical data |
|---|---|---|---|
| 85 | 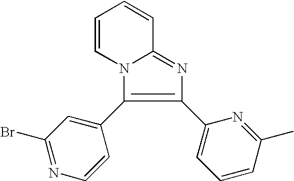 | 40 | ¹H NMR (300 MHz, CDCl₃, ppm) δ: 8.47 (d, 1H), 8.12 (d, 1H), 7.8 (m, 2H), 7.7 (d, 1H), 7.6 (t, 1H), 7.47 (d, 1H), 7.29 (t, 1H), 7.05 (d, 1H), 6.85 (t, 1H), 2.39 (s, 3H) |
| 86 | 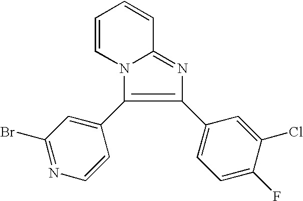 | 41 | APCI MS m/z 404 (MH⁺) |
| 87 | 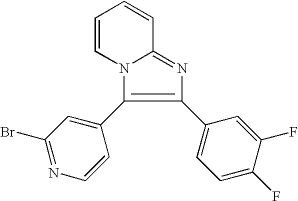 | 42 | APCI MS m/z 386 (MH⁺) |
| 88 | 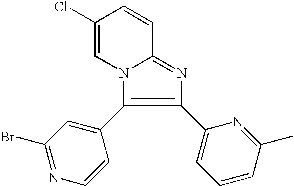 | 40 | ¹H NMR (300 MHz, CDCl₃, ppm) δ: 8.5 (d, 1H), 8.09 (d, 1H), 7.82 (s, 2H), 7.65 (t, 2H), 7.45 (d, 1H), 7.27 (d, 1H), 7.08 (d, 1H), 2.39 (s, 3H) |
| 89 | 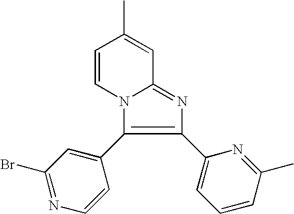 | 40 | ¹H NMR (300 MHz, CDCl₃, ppm) δ: 8.43 (d, 1H), 8.00 (d, 1H), 7.82 (s, 1H), 7.78 (d, 1H), 7.60 (t, 1H), 7.44 (m, 2H), 7.05 (d, 1H), 6.70 (d, 1H), 2.43 (s, 3H), 2.40 (s, 3H) |
| 90 | 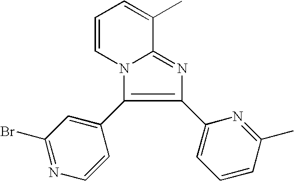 | 40 | ¹H NMR (300 MHz, CDCl₃, ppm) δ: 8.55 (d, 1H), 8 (d, 1H), 7.85 (d, 1H), 7.8 (s, 1H), 7.65 (t, 1H), 7.45 (d, 1H), 7.1 (m, 2H), 6.8 (t, 1H), 2.7 (s, 3H), 2.4 (s, 3H) |
| 91 | 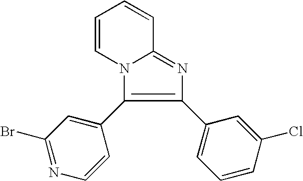 | 43 | APCI MS m/z 384 (MH+) |
| 92 | 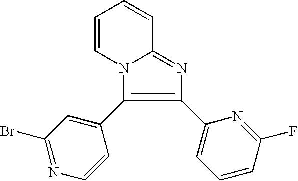 | 44 | APCI MS m/z 369 (MH+) |

Intermediate 93: 4-{4-[3-(6-Methylpyridin-2-yl)-1-trityl-1H-pyrazol-4-yl]pyridin-2-yl}-phenol

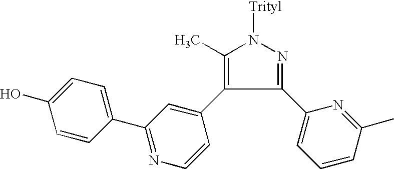

To a solution of intermediate 82 (2 g, 3.6 mmol) in a mixture of DME (36 ml) and water (18 ml) were added tetrakis-triphenylphosphine palladium (0.2 g), Na₂CO₃ (0.99 g) and 4-hydroxyphenyl boronic acid, pinacol ester (1.4 eq, 1.15 g, 4.32 mmol) and the resulting mixture was heated under reflux overnight. The cooled mixture was poured into water and extracted with CH₂Cl₂. The organic phase was washed with water, dried over Na₂SO₄ and filtered. Evaporation of the solvent in vacuo gave a crude oil which was purified by chromatography on silica gel (CH₂Cl₂/MeOH 95:5) to give the title compound as a white solid (1.7 9 g, 83%), which contained the 2-trityl isomer as a minor component; [APCl MS] m/z 571 (MH+).
Intermediate 94: 4-{4-[3-(6-Methylpyridin-2-yl)-1-trityl-1H-pyrazol-4-yl]pyridin-2-yl}-benzoic Acid 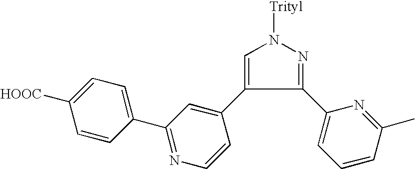
Intermediate 82 (1 g, 1.8 mmol) and 4-carboxybenzene boronic acid (0.36 g, 2.52 mmol) were coupled and treated as described for intermediate 93 to afford the title compound as a white solid (600 mg, 61%) containing the 2-trityl isomer as a minor component; [APCl MS] m/z 599 (MH+).
Intermediate 95: 4-{4-[3-(6-methylpyridin-2-yl)-1-trityl-1H-pyrazol-4-yl]pyridin-2-yl}benzaldehyde

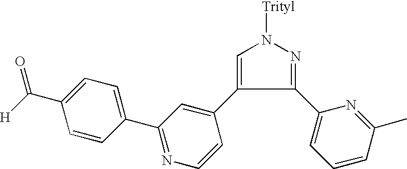

Intermediate 82 (1 g, 1.8 mmol) and 4formylphenylboronic acid (0.35 g, 2.3 mmol) were coupled and treated as described for intermediate 93 to afford the title compound as a grey solid (1 g, 96%) containing the 2-trityl isomer as a minor component; [APCl MS] m/z 583 (MH+).
Intermediate 96: 2-(4-bromophenyl)-4-[3-(6-methylpyridin-2-yl)-1-(triphenylmethyl)-1H-pyrazol-4-yl]pyridine

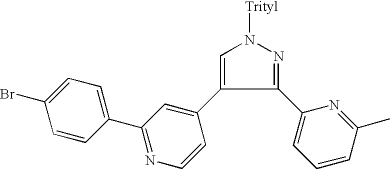

Intermediate 82 (2 g, 3.6 mmol) and 4-bromophenylboronic acid (0.755 g, 3.78 mmol) were coupled and treated as described for intermediate 93 to afford the title compound as a white solid (2.1 g, 92%) containing the 2-trityl isomer as a minor component; [APCl MS] m/z 633/635 (MH+).
Intermediate 97: (4-{4-[3-(6-methylpyridin-2-yl)-1-(triphenylmethyl)-1H-pyrazol-4-yl-pyridin-2-yl}phenyl)amine

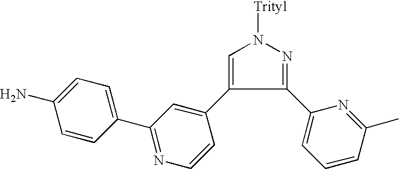

Intermediate 82 (1 g, 1.8 mmol) and 4-(4,4,5,5-tetramethyl-1,3,2-dioxaborolan-2-yl)aniline (0.512 g, 2.3 mmol) were coupled and treated as described for intermediate 93 to afford the title compound as a yellow solid (1 g, 98%) containing the 2-trityl isomer as a minor component; [APCl MS] m/z 570 (MH⁺).
Intermediate 98: 4-{4-[5-methyl-3-(6-methylpyridin-2-yl)-1-(triphenylmethyl)-1H-pyrazol-4-yl]-2-pyridinyl}benzaldehyde

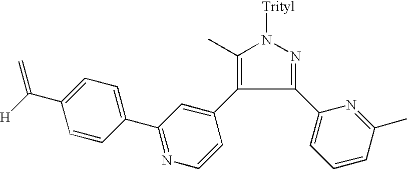

Intermediate 83 (1.5 g, 2.62 mmol) and 4-formylphenylboronic acid (0.48 g, 3.2 mmol) were coupled and treated as described for intermediate 93 to afford the title compound as a yellow oil (1.6 g, quantitative) containing the 2-trityl isomer as a minor component; [APCl MS] m/z 355 (MH⁺, loss of trityl).
Intermediate 99: 3-(2-(4-formyl-phenyl)-pyridin-4-yl)-2-(6-methyl-pyridin-2-yl)-imidazo[1,2-a]pyridine

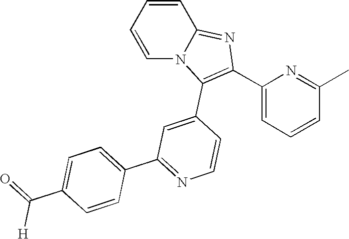

A solution of intermediate 85 (500 mg, 1.37 mmol) in DME (50 mL) was treated with tetrakis (triphenylphosphine)palladium(0) (158 mg, 10%mol) and stirred at room temperature for 30 min. Na₂CO₃ (2M) (4.2 ml) was added to the reaction mixture, followed by 4-formylphenyl boronic acid (267 mg, 1.78 mmol). The resulting mixture was heated under reflux overnight. The cooled mixture was poured into ice and extracted with CH₂Cl₂. The organic phase was washed with water, dried over Na₂SO₄ and filtered. Evaporation of the solvent in vacuo gave a crude oil which was purified by chromatography on silica gel (CH₂Cl₂/MeOH 95:5). The title compound was obtained as a cream powder (310 mg, 58%); ¹H NMR (300 MHz, CDCl₃, ppm) δ: 10.08 (s, 1H) ; 8.86 (d, 1H); 8.10-8.20 (m, 4H); 7.98 (d, 1H); 7.83 (d, 1H); 7.75 (d, 1H); 7.61 (t, 1H); 7.51 (m, 1H); 7.30 (t, 1H); 7.04 (d, 1H) ; 6.85(t, 1H); 2.31 (s, 3H).

The following intermediates (see Table 5) were prepared by methods analogous to that described for intermediate 99.

TABLE 5

| Int. | structures | From int. | Physical data |
|---|---|---|---|
| 100 | 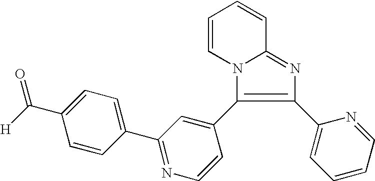 | 84 | APCI MS m/z 377 (MH+) |
| 101 | 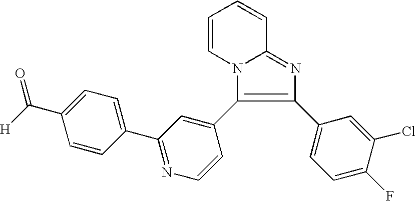 | 86 | APCI MS m/z 428 (MH+) |
| 102 | 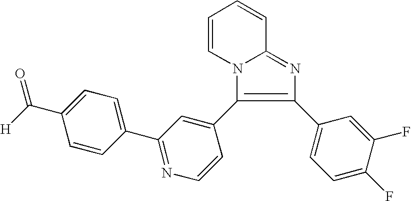 | 87 | APCI MS m/z 412 (MH+) |
| 103 | 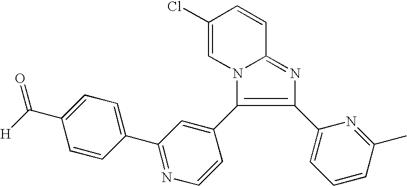 | 88 | APCI MS m/z 425 (MH+) |
| 104 | 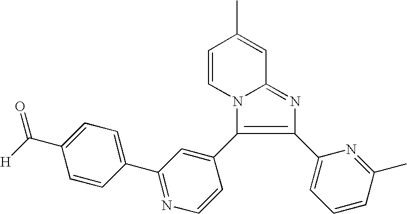 | 89 | APCI MS m/z 405 (MH+) |
| 105 | 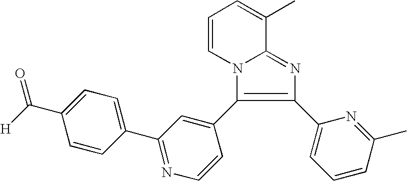 | 90 | ¹H NMR (300 MHz, CDCl₃, ppm) δ: 10.1 (s, 1H), 8.9 (d, 1H), 8.2 (d, 2H), 8.15 (s, 1H), 8.1 (d, 1H), 8 (d, 2H), 7.85 (d, 1H), 7.6 (m, 2H), 7.1 (m, 2H), 6.8 (t, 1H), 2.8 (s, 3H), 2.4 (s, 3H) |
| 106 | 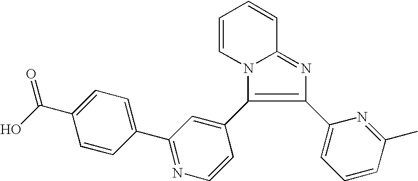 | 85 | APCI MS m/z 407 (MH+) |
| 107 | 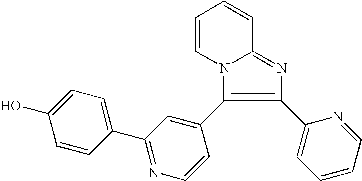 | 84 | APCI MS m/z 365 (MH+) |
| 108 | 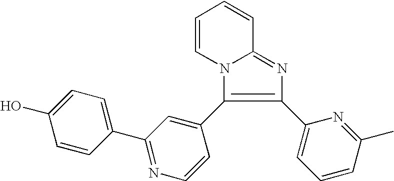 | 85 | APCI MS m/z 379 (MH+) |
| 109 | 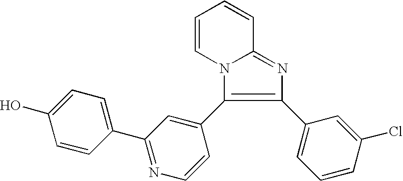 | 91 | ¹H NMR (300 MHz, CDCl₃, ppm) δ: 9.05 (s, 1H), 8.55 (d, 1H), 7.95 (d, 1H), 7.6 (m, 3H), 7.5 (m, 2H), 7.25 (m, 1H), 7.1 (m, 4H), 6.7 (m, 3H) |
| 110 | 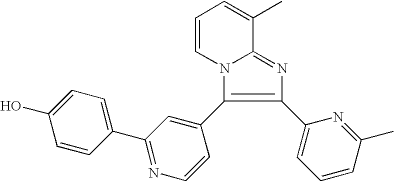 | 90 | APCI MS m/z 393 (MH+) |
| 111 | 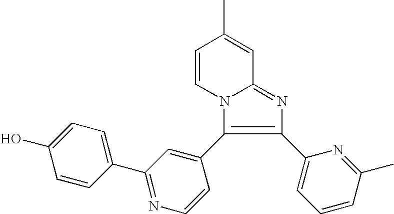 | 89 | APCI MS m/z 393 (MH+) |
| 112 | 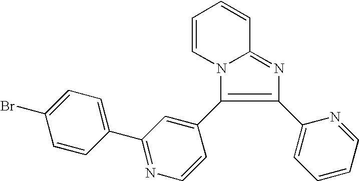 | 84 | APCI MS m/z 428 (MH+) |

Intermediate 113: 3-{2-[4-(2-bromo-ethoxy)-phenyl]-pyridin-4-yl}-2-pyridin-2-yl-imidazo[1,2-a]pyridine

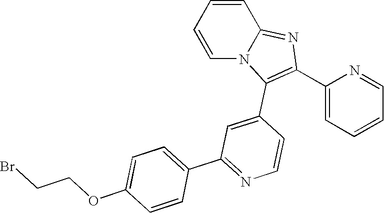

To a solution of intermediate 107 (0.38 g, 1.04 mmol) in acetone (20 ml) was added cesium carbonate (0.68 g, 2.08 mmol) and 1,2-dibromoethane (0.9 ml, 10.4 mmol) and the mixture was heated under reflux for 2 days. After cooling, the reaction was filtered and the solvent was removed in vacuo. After purification by chromatography on silica gel (CH₂Cl₂/MeOH, 90:10), the title compound was obtained as a yellow gum (140 mg, 28%); ¹H NMR (CDCl₃, 300 MHz) δ ppm: 8.78 (d, 1H), 8.49 (d, 1H), 8.14 (d, ₁H), 7.93 (m, 4H), 7.72 (t, 2H), 7.34 (m, 2H), 7.17 (m, 1H), 7.00 (d, 2H), 6.83 (t, 1H), 4.33 (t, 2H), 3.65 (t, 3H).

The following intermediates (see Table 6) were prepared by methods analogous to that described for intermediate 113.

TABLE 6

| | | From | |
|---|---|---|---|
| Int. | structures | int. | Physical data |
| 114 | 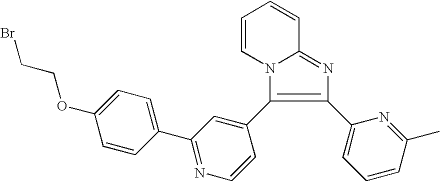 | 108 | ¹H NMR (300 MHz, CDCl₃, ppm) δ: 8.75 (d, 1H), 8.15 (d, 1H), 7.93 (m, 3H), 7.71 (t, 2H), 7.56 (t, 2H), 7.35 (d, 1H), 7.26 (m, 1H), 7.00 (m, 3H), 6.82 (t, 1H), 4.33 (t, 2H), 3.65 (t, 2H), 2.37 (s, 3H) |
| 115 | 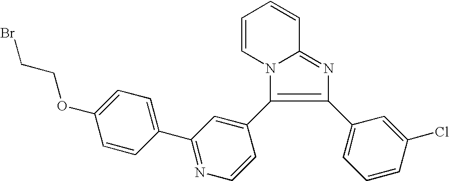 | 109 | APCI MS m/z 505 (MH+) |
| 116 | 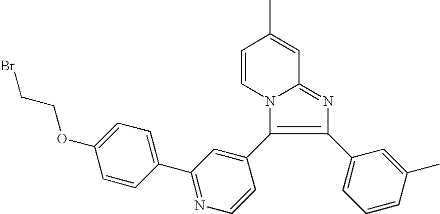 | 111 | APCI MS mlz 500 (MH+) |
| 117 | 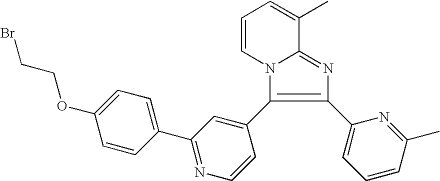 | 110 | ¹H NMR (300 MHZ, CDCl₃, ppm) δ: 8.75 (d, 1H), 8.05 (d, 1H), 7.95.(s, 1H), 7.9 (d, H), 7.7 (d, 1H), 7.55 (t, 7.35 (d, 1H), 7 (m, 2H), 6.9 (d, 2H), 6.75 (t, 1H), 4.35 (t, 2H), 3.65 (t, 2H , 2.75 (s, 3H), 2.35 (s, 3H) |

Intermediate 118: 2-Hydroximino-2-[2-Bromo-pyridin-4-yl]-1-(6-methyl-pyridin-2-yl)-ethan-1-one

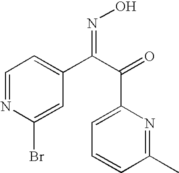

A solution of intermediate 40 (20 g, 68.7 mmol) in aqueous HCl 18% (360 ml) was cooled to 0° C. using a dry ice bath. To this solution was added sodium nitrite (5.6 g, 82.44 mmol), the reaction temperature was maintained at 0° C. during this addition. After addition was complete, the dry ice bath was removed and the reaction allowed to warm and stirred at room temperature for 30 min. The reaction mixture was basified with aqueous NaOH 35%. The resulting precipitate was filtered, washed with water and dried to give the title compound (mixture of two isomers) as a pink solid (20.53 g, 93%). This compound was used in the next step without purification; [APCl MS] m/z 321 (MH+).
Intermediate 119: 2-Hydroximino-2-[2-Bromo-pyridin-4-yl]-1-(pyridin-2-yl)-ethan-1-one

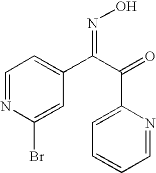

The title compound was obtained from intermediate 39, as described for intermediate 118, as a solid (36 g, 98%); m.p. 200° C.; [APCl MS] m/z 307 (MH+).
Intermediate 120: 2-tert-butyl-4-(6-methyl-pyridin-2-yl)-5-(2-bromo-pyridin-4-yl)-imidazole

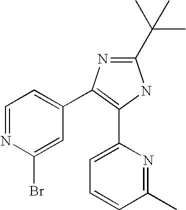

Intermediate 118 (6 g, 18.7 mmol) was dissolved in acetic acid (50 mL) and treated with ammonium acetate (4.33 g, 56.1 mmol) and pivalaldehyde (2.7 g, 37.4 mmol). The resulting mixture was heated at reflux for 1 hour, then allowed to cool at room temperature and was concentrated. The residue was dissolved into water and extracted with CH₂Cl₂. The organic phase was dried over Na₂SO₄ and evaporated to dryness under reduced pressure to give 2-tert-butyl-4-(6-methyl-pyridin-2-yl)-N-1-hydroxy-5-(2-bromo-pyridin-4-yl)-imidazole; (6.31 g, 87%). 2-tert-Butyl-4-(6-methyl-pyridin-2-yl)-N-1-hydroxy-5-(2-bromo-pyridin-4-yl)-imidazole (6.31 g, 16.21 mmol) was dissolved in DMF (60 mL), treated with triethyl phosphite (2.78 mL, 16.21 mmol) and the resulting mixture was heated at 130° C. for 5 h. To complete the reaction triethyl phosphite (0.2 eq) was added and the mixture was stirred at 130° C. for 18 h. The reaction mixture was poured into water and extracted with EtOAc. The organic layer was washed with water, dried over Na₂SO₄ and concentrated under reduced pressure. The crude oil was precipitated with diisopropyl ether to afford the title compound as a brown solid (3.88 g, 65%); m.p. 200° C.; [APCl MS] m/z 372 (MH+).

The following compounds of formula (IVDa) were prepared by methods analogous to that described for intermediate 120 using the starting materials indicated (see Table 7).

TABLE 7

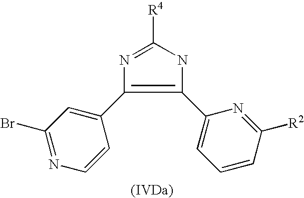

| Int. | R² | R⁴ | From Int. | Physical data |
|---|---|---|---|---|
| 121 | H | t-butyl | 119 | [APCI MS] m/z 358 (MH+) |
| 122 | methyl | i-propyl | 118 | [APCI MS] m/z 358 (MH+) |
| 123 | H | i-propyl | 119 | [APCI MS] m/z 343/345 (MH+) |
| 124 | methyl | methyl | 118 | [APCI MS] m/z 330 (MH+) |

Intermediate 125: 4-{4-[2-tert-Butyl-5-(6-methyl-pyridin-2-yl)-1H-imidazol4-yl]-pyridin-2-yl}-benzaldhyde

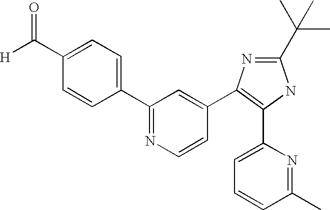

Intermediate 120 (2 g, 5.4 mmol) and 4-formylphenyl boronic acid (1.13 g, 7.56 mmol) were reacted as described for intermediate 99 to afford the title compound as a yellow solid (2.37 g, quantitative); [APCl MS] m/z 397 (MH+).
Intermediate 126: 4-{4-[2-tert-Butyl-5-(6-methyl-pyridin-2-yl)-1H-imidazol-4-yl]-pyridin-2-yl}-phenol

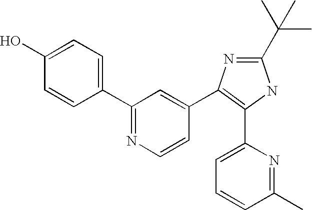

Intermediate 120 (2.5 g, 6.7 mmol) and 4-hydroxyphenyl boronic acid (1.3 g, 9.38 mmol) were reacted as described for intermediate 99, to afford the title compound as a brown solid (1.57 g, 61%); ¹H NMR (350 MHz; CDCl₃, ppm) δ: 8.37 (1H, d), 7.70 (1H, s), 7.46 (2H, d), 7.30 (1H, t), 7.20-7.10 (2H, m), 6.83(1H, d), 6.60(1H, d), 3.85-3.23 (4H, brd), 2.27(3H, s), 1.27 (9H, s); [APCl MS] m/z 385 (MH+).
Intermediate 127: 4-{4-[2-isopropyl-5-(6-methyl-pyridin-2-yl)-1H-imidazol-4yl]-pyridin-2-yl}-benzaldhyde

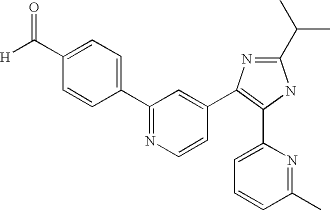

Intermediate 122 (2.05 g, 5.74 mmol) and 4-formylphenyl boronic acid (1.2 g, 8.04 mmol) were reacted as described for intermediate 99 to afford the title compound as a pale yellow solid (1.22 g, 56%); ¹H NMR (300 MHz; CDCl₃, ppm) δ: 9.90 (1H, s), 8.54 (1H, d), 8.02 (2H, d), 7.79 (2H, d), 7.47-7.16 (3H, m), 6.87 (1H, d), 3.12-2.95 (1H, m), 2.39(3H, s), 1.25 (6H, d); [APCl MS] m/z 383 (MH+).
Intermediate 128: Polymer supported 5-(2-bromo-4-pyridinyl)-4-(2-pyridinyl)-1,3-thiazol-2-amine

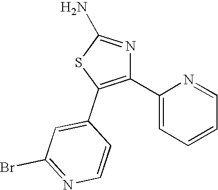

Step 1: Rink Argopore resin (12 g, 0.58 mmol/g substitution) was placed into a peptide vessel and washed with CH₂Cl₂ (3×100 ml). The resin was then treated for 10 min with a solution of piperidine 20% in DMF (3×40 mL). After washing with DMF (3×100 mL) and CH₂Cl₂ (3×100 mL), the resin was treated with a solution of Fmoc-NCS (0.2M) in CH₂Cl₂ (170 mL) under argon at room temperature for 1 h. The resin was washed with DMF (3×100 mL), EtOH (3×100 mL) and CH₂Cl₂ (3×100 mL) and subsequently stirred for 10 min with a solution of piperidine 20% in DMF (3×40 mL) to give after washing with DMF (3×100 mL) and CH₂Cl₂ (3×100 mL) the resin bound thiourea.

Step 2: To a solution of intermediate 39 (8.5 g, 29 mmol) in dioxane (145 mL) was added under argon polymer-supported pyridinium perbromide (1.8 mmol/g, 16 g). The suspension was shaken under argon at room temperature overnight The resin was removed by filtration and washed with dioxane (25 mL) to give 2-bromo-2-(2-bromo-4-pyridinyl)-1-(2-pyridinyl)ethanone which was used in solution in dioxane without purification in the next step.

Step 3: The product from step 1 was stirred with the product from step 2 (0.18 M) in dioxane (175 mL) for 4 h at room temperature under argon. The resin was washed with dioxane (3×100 mL). A second exposure with the product from step 2 (0.18M in dioxane, 175 mL) was performed. The resin was washed with DMF (3×100 mL), EtOH (3×100 mL), CH₂Cl₂ (3×100 mL) and dried under a stream of nitrogen overnight. 2 mg of the obtained resin were cleaved with a solution of TFA 20% in CH₂Cl₂ to give the title compound which was characterised by LC-MS (purity>96%); [APCl MS] m/z 333, 335, 336 (MH+).
Intermediate 129: Polymer supported 5-(2-bromo-4-pyridinyl)-4-(6-methyl-2-pyridinyl)-1,3-thiazol-2-amine

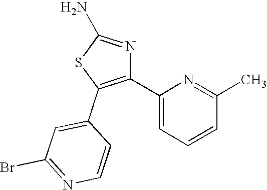

Intermediate 129 was prepared in analogous fashion to intermediate 128 starting from intermediate 40. After step 3, 2 mg of the obtained resin were cleaved with a solution of TFA 20% in CH₂Cl₂ to give the title compound which was characterised by LC-MS (purity>96%); [APCl MS] m/z 347/349/350 (MH+).

### PYRAZOLE EXAMPLES

### Example 1

### 2-{4-[(1-methyl-1H-imidazol-4-yl)methoxy]phenyl}-4-[3-(6-methylpyridin-2-yl)-1H-pyrazol-4-yl]pyridine

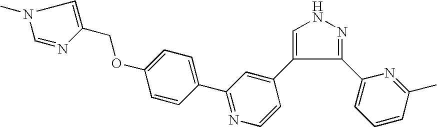

To an ice-cooled solution of intermediate 93 (4 g, 7 mmol) in DMF (80 ml) was added portionwise sodium hydride (0.6 g, 3 eq, 21 mmol) and the mixture then stirred at room temperature for 30 mins. Intermediate 22 (1.6 g, 10 mmol) was added and the mixture stirred at room temperature overnight and then poured into water and extracted with CH₂Cl₂. The organic layer was dried over Na₂SO₄ and filtered. Evaporation of the solvent in vacuo gave a crude oil which was purified by chromatography on silica gel (CH₂Cl₂/MeOH 97:3) to give the trityl compound as an oil (3 g). This compound was dissolved in methanol (60 ml) and HCl (1N, 40 ml) and the solution was heated under reflux for 2 hours and then concentrated in vacuo. The residue was dissolved in water and washed with CH₂Cl₂. The aqueous layer was basified with NaOH (1N) and extracted with CH₂Cl₂. The organic extract was washed with water and dried over Na₂SO₄, filtered and evaporated to give a solid which was crystallised from EtOH to give the title compound as white crystals (1.1 g, 37%); m.p. 191° C.; TOF MS ES⁺ exact mass calculated for C₂₅H₂₂N₆O: 423.1933 (MH+). Found: 423.1928 (MH+).

### Example 2

### 2-[4-(Ethylsulfonyl)phenyl]-4-[3-(6-methylpyridin-2-yl)-1H-pyrazol-4-yl]pyridine

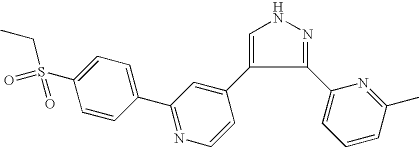

To a solution of intermediate 82 (0.5 g, 0.9 mmol) in a mixture of DME (18 ml) and water (9 ml) was added 4-(ethylsulfonyl)phenyl boronic acid (1.3 eq, 0.25 g, 1.17 mmol), tetrakis (triphenylphosphine)palladium(0) (0.05 g) and Na₂CO₃ (3 eq, 0.28 g, 2.69 mmol) and the reaction mixture was heated under reflux overnight. The cooled mixture was poured into ice and extracted with CH₂Cl₂. The organic layer was washed with water, dried over Na₂SO₄ and filtered. Evaporation of the solvent in vacuo gave an oil which was dissolved in MeOH (30 ml) and HCl (1N, 20 ml). The solution was heated under reflux for 3 hours and then concentrated under reduced pressure. The residue was dissolved in water and washed with CH₂Cl₂. The aqueous layer was basified with NaOH (1N) and extracted with CH₂Cl₂. The organic extract was washed with water, dried over Na₂SO₄, filtered and evaporated under reduced pressure. After chromatography on silica gel (CH₂Cl₂/MeOH, 95:5) and crystallisation from DMF, the title compound was obtained as white crystals (166 mg, 45.7%); m.p. 244° C.; [APCl MS] m/z 405 (MH+).

The following compounds of formula (IAb) were prepared by methods analogous to that described for Example 2 using the starting materials indicated (see Table 9).

TABLE 9

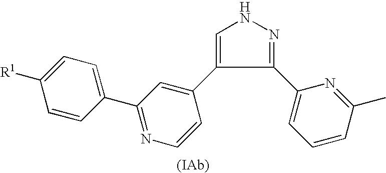

| Ex | R¹ | From Int. | Physical data |
|---|---|---|---|
| 3 | cyano | 82 | TOF MS ES⁺ exact mass calculated |
| | | | for C₂₁H₁₅N₅(MH+): 338.1406, |
| | | | found: 338.1408; m.p. 198° C. |
| 4 | trifluoromethoxy | 82 | TOF MS ES⁺ exact mass calculated |
| | | | for C₂₁H₁₅F₃N₄O(MH+): 397.1276, |
| | | | found: 397.1269; m.p. 131° C. |
| 5 | chloro | 82 | TOF MS ES⁺ exact mass calculated |
| | | | for C₂₀H₁₅CIN₄(MH+): 347.1063, |
| | | | found: 347.1057; m.p. 190° C. |
| 6 | methoxy | 82 | TOF MS ES⁺ exact mass calculated |
| | | | for C₂₁H₁₈N₄O(MH+): 343.1600, |
| | | | found: 343.1600 |
| 7 | methanesulfonyl | 82 | ¹H NMR (300 MHz, CDCl₃, ppm) δ: |
| | | | 8.84 (d, 1H), 8.52 (d, 2H), 8.44 (s, |
| | | | 1H), 8.26 (d, 2H), 8.01 (t, 1H), 7.89- |
| | | | 7.68 (m, 2H), 7.50 (d, 1H), 3.51 (s, |
| | | | 3H), 2.64 (s, 3H); m.p. 232-234° C. |

### Example 8

### 4-[3-(6-methylpyridin-2-yl)-1H-pyrazol-4-yl]-2-[4-(pyrrolidin-1-ylmethyl)phenyl]pyridine

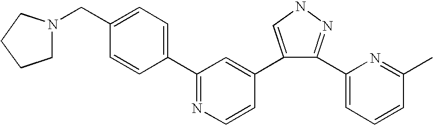

To a solution of intermediate 95 (0.29 g, 0.5 mmol) and pyrrolidine (4 eq, 0.142 g) in dry dichloroethane (20 mL) was added acetic acid (1.5 eq, 0.05 g) followed by sodium triacetoxyborohydride (2 eq, 0.224 g). The mixture was stirred at room temperature overnight, diluted with water, extracted with CH₂Cl₂ and dried over Na₂SO₄ . The solvent was removed under reduced pressure and the resulting product was treated with a mixture of MeOH/HCl 1N (3:2, 50 ml) at reflux for 3 h. The reaction mixture was concentrated to dryness to give a residue which was dissolved in water and washed with CH₂Cl₂. The aqueous phase was basified with NaOH (1N), extracted with CH₂Cl₂ and dried over Na₂SO₄. Concentration to dryness gave a solid which was precipitated from a mixture CH₂Cl_{2/}hexane to give the title compound as (0.095 g, 48%); ¹H NMR (CDCl₃) δ 8.62 (d, 1H); 7.88 (d, 2H); 7.71 (d, 2H); 7.50-7.39 (m, 3H); 7.26-7.20 (m, 2H); 7.05 (d, 1H); 3.76 (brs, 2H); 2.79-2.56 (m, 4H); 2.53 (s, 3H); 1.91-1.75 (m, 4H); TOF MS ES⁺ exact mass calculated for C₂₅H₂₅N₅: 396.2188(MH+):. Found: 396.2174(MH+).

The following compounds of formula (IAa) were prepared by methods analogous to that described for Example 8 using the starting materials indicated (see Table 10).

TABLE 10

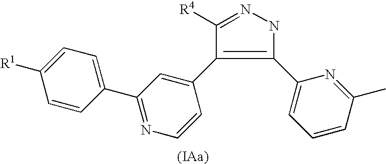

| | | | From | |
|---|---|---|---|---|
| Ex | R¹ | R⁴ | Int. | Physical data |
| 9 | (morpholin-4- | H | 95 | TOF MS ES⁺ exact mass calculated for |
| | yl)methyl | | | C₂₅H₂₅N₅O(MH+): 412.2137, found: |
| | | | | 412.2150; ¹H NMR (300 MHz, CDCl₃, ppm) δ: |
| | | | | 8.7 (d, 1H), 7.98 (d, 2H), 7.81 (d, 2H), 7.58- |
| | | | | 7.41 (m, 3H), 7.37-7.3 (m, 2H), 7.14 (d, 1H), |
| | | | | 3.89-3.7 (m, 4H), 3.62 (brs, 2H), 2.62 (s, 3H), |
| | | | | 2.6-2.47 (m, 4H) |
| 10 | (N-methyl-2- | H | 95 | ¹H NMR (300 MHz, CDCl₃, ppm) δ: 8.59 (d, |
| | methoxyethylamino) | | | 1H), 7.83 (d, 2H), 7.71 (s, 1H), 7.66 (s, 1H), |
| | methyl | | | 7.47-7.29 (m; 3H), 7.24-7.18 (m, 2H), 7.03 (d, |
| | | | | 1H), 3.59-3.5 (m, 2H), 3.45 (brt, 2H), 3.25 (s, |
| | | | | 3H), 2.56 (brt, 3H), 2.60-2.47 (m, 4H), 2.5 (s, |
| | | | | 3H) |
| 11 | (4-methoxy- | H | 95 | TOF MS ES⁺ exact mass calculated for |
| | piperidin-1- | | | C₇H₂₉N₅O: 440.2450(MH+), found: |
| | yl)methyl | | | 440.2438(MH+); ¹H NMR (300 MHz, CDCl₃, |
| | | | | ppm) δ:8.64 (d, 1H), 7.88 (d, 2H), 7.76 (s, |
| | | | | 1H), 7.72 (s, 1H), 7.47, (t, 1H), 7.44-7.35 (m, |
| | | | | 2H), 7.29-7.23 (m, 2H), 7.09 (d, 1H), 3.61- |
| | | | | 3.50 (m, 2H), 3.29 (s, 3H), 3.26-3.14 (m, 1H), |
| | | | | 2.81-2.66 (m, 2H), 2.56 (5, 3H), 2.30-2.08 (m, |
| | | | | 2H), 2.01-1.82 (m, 2H), 1.69-1.52 (m, 2H) |
| 12 | (morpholin-4- | methyl | 98 | [APCI MS] m/z 426 (MH+) |
| | yl)methyl | | | ¹H NMR (300 MHz, CDCl₃, ppm) δ: 8.66 (d, |
| | | | | 1H), 7.86 (d, 2H), 7.64 (s, 1H), 7.44-7.30 (m, |
| | | | | 3H), 7.15-7.11 (m, 1H), 7.03-6.89 (m, 2H), |
| | | | | 3.74-3.60 (m, 4H), 3.51 (brs, 2H), 2.50 (s, |
| | | | | 3H), 2.40-2.34 (m, 4H), 2.25 (s, 3H) |

### Example 13

### 4-(4-{4-[3-(6-methylpyridin-2-yl)-1H-pyrazol-4-yl]pyridin-2-yl}benzoyl)morpholine

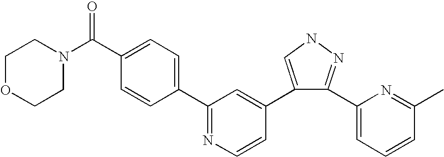

To a solution of intermediate 94 (0.2 g, 0.34 mmol, 1 eq.) in CH₂Cl₂ (50 mL) were added morpholine (0.035 g, 0.4 mmol), HOBT (0.59 g, 1.3 eq.), EDCl (0.83 g, 1.3 eq.) and Et₃N (0.04 g, 2.3 eq.) and the reaction mixture was stirred at room temperature overnight. The reaction was hydrolysed and extracted with CH₂Cl₂. The solvent was removed under reduced pressure. The residue was treated with MeOH/HCl 1N (3/2, 30 ml) at reflux for 1 h. After removal of the solvent under reduced pressure, the residue was dissolved in water and washed with CH₂Cl₂. The aqueous phase was basified with NaOH 1N and extracted with CH₂Cl₂ The organic phase was dried, filtered, and evaporated to dryness to give a crude solid which was precipitated with a mixture CH₂Cl_{2/}hexane to give the title compound (0.075 g; 52%); ¹H NMR (300 MHz, CDCl₃) δ 8.63 (1H, d); 7.95 (2H, d); 7.76 (2H, s); 7.52-7.42 (3H, m); 7.27 (2H, d); 7.12-7.01 (1H, m); 3.50-3.32 (8H, m); 2.51 (3H, brs) ; TOF MS ES⁺ exact mass calculated for C₂₅H₂₃N₅O₂: 426.1930(MH+). Found: 426.1931(MH+).

### Example 14

### 4-{4-[3-(6-methylpyridin-2-yl)-1H-pyrazol-4-yl]pyridin-2-yl}-N-(tetrahydro-2H-pyran-4-yl)benzamide

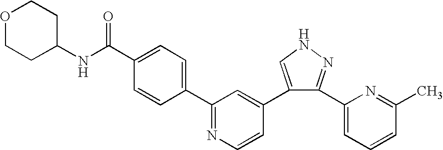

Intermediate 94 (0.4 g, 0.67 mmol) and 4-aminotetrahydropyran (0.081 g, 0.8 mmol) were reacted as was described for example 13 to give the title compound (0.2 g, 68%); m.p. 148° C.; TOF MS ES⁺ exact mass calculated for C₂₆H₂₅N₅O₂: 440.2086(MH+). Found: 440.2060(MH+).

### Example 15

### N-(4-{4-[3-(6-methylpyridin-2-yl)-1H-pyrazol-4-yl]pyridin-2-yl}phenyl)-2-morpholin-4-ylacetamide

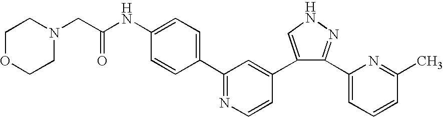

Intermediate 97 (0.38 g, 0.67 mmol) and 4-morpholinylacetic acid hydrochloride (0.156 g, 0.86 mmol) were reacted as was described for example 13 to give the title compound as an off-white solid (0.115 g, 38%); ¹H NMR (300 MHz, CDCl₃) δ 9.10 (1H, s); 8.58(1H, d); 7.87 (2H, d); 7.70 (2H, s); 7.59 (2H, d); 7.28-7.18 (2H, m); 7.10-6.97 (1H, m); 3.71 (4H, t); 3.08 (3H, s); 2.56 (4H, t); 2.50 (3H, s); TOF MS ES⁺ exact mass calculated for C₂₆H₂₆N₆O₂: 455.2195 (MH+). Found: 455.2195 (MH+).

### Example 16

### 4-(4-{4-[3-(6-methylpyridin-2-yl)-1H-pyrazol-4-yl]pyridin-2-yl}phenyl)morpholine

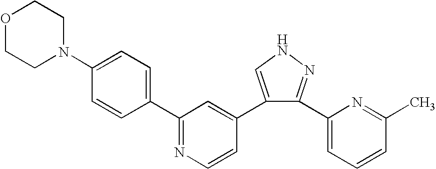

Step 1: To a solution of intermediate 96 (0.633 g, 1 mmol) in toluene (10 ml) were added morpholine (0.348 g, 4 mmol, 4 eq), Pd₂(dba)₃ (0.045 g, 0.049 mmol, 0.05 eq), binap (0.062 g, 0.1 mmol, 0.1 eq) and t-BuOK (0.134 g, 1.4 mmol, 1.4 eq) and the reaction mixture was refluxed for 5 hours. The mixture was then poured into ice and extracted with EtOAc. The organic phase was washed with water and dried over Na₂SO₄. Concentration to dryness gave a crude product that was purified by chromatography on silica gel (CH₂Cl₂/CH₃OH 98:2) to afford 4-(4-4-{4-[3-(6-methyl-2-pyridinyl)-1-(triphenylmethyl)-1H-pyrazol-4-yl]-2-pyridinyl}phenyl)morpholine.

Step 2: 4-(4-[3-(6-Methyl-2-pyridinyl)-1-(triphenylmethyl)-1H-pyrazol-4-yl]-2-pyridinyl}phenyl)morpholine was treated with a mixture of MeOH/HCl 1N (3:2, 50 ml) under reflux for 2 hours. The reaction mixture was poured into water and extracted with CH₂Cl₂. The aqueous phase was basified with NaOH (1N) and extracted with CH₂Cl₂. The organic phase was washed with water, dried and evaporated to dryness to give a crude product which was precipitated with a mixture of CH₂Cl₂/hexane to afford the title compound as a yellow solid (0.31 g, 78%); TOF MS ES⁺ exact mass calculated for C₂₄H₂₃N₅O: 398.1981 (MH+). Found 398.1961(MH+).

### Example 17

### 2-[-4-(2-methyl-1H-imidazol-1-yl)phenyl]-4-[3-(6-methylpyridin-2-yl)-1H-pyrazol-4-yl]pyridine

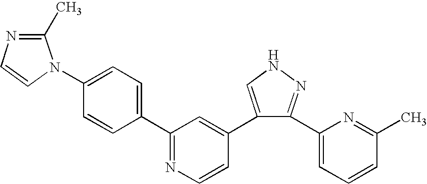

Intermediate 96 (0.308 g, 0.5 mmol) and 2-methyl-1H-imidazole (0.82 g, 1 mmol) were reacted as described for example 16 to give the title compound as a white solid (0.013 g, 6%); m.p. 128° C.; ¹H NMR (300 MHz; CDCl₃) δ: 8.64 (1H, d), 8.02 (2H, d), 7.75 (2H, d), 7.48 (1H, t), 7.37-7.20 (4H, m), 7.19 (1H, s), 7.08 (1H, d), 6.99 (2H, d), 2.52 (3H, s), 2.35 (3H, s).

### Example 18

### 3-[2-(4-((tetrahydropyran-4-yl)aminocarbonyl)phenyl)pyridin-yl]-4-[6-methylpyridin-2-yl]-1H-pyrazole

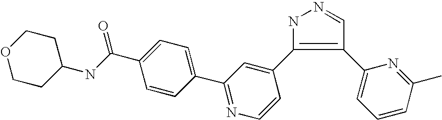

To a solution of intermediate 71 (0.6 g, 1.44 mmol) in DMF (10 m) and acetic acid (0.2 ml, 3.47 mmol) was added DMF.DMA (0.258 g, 2.16 mmol) and the mixture was stirred at room temperature for 2 h. Hydrazine hydrate (3 ml) was added and the mixture was stirred at room temperature overnight, then was heated at 40° C. for 2 h and then poured into water. The aqueous phase was extracted with CH₂Cl₂, the organic phase dried over Na₂SO₄ and concentrated under reduced pressure. The residue was purified by chromatography on silica gel, eluting with CH₂C₂/MeOH (95/5). After crystallisation from EtOAc, the title compound was obtained as white crystals (0.2 g, 31.51%); m.p. 170° C.; TOF MS ES⁺ exact mass calculated for C₂₆H₂₅N₅O₂: 440.2086 (MH+). Found 440.2065 (MH+).

The following compounds of formula (IAb) were prepared by methods analogous to that described for Example 18 using the starting materials indicated (see Table 11).

TABLE 11

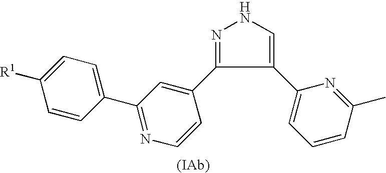

| | | From | |
|---|---|---|---|
| Ex | R¹ | Int. | Physical data |
| 19 | (4-ethylpiperazin-1- | 70 | TOF MS ES⁺ exact mass |
| | yl)carbonyl | | calculated for C₂₇H₂₈N₆O(MH+): |
| | | | 453.2403, found: 453.2394 |
| | | | m.p. 126° C. |
| 20 | morpholin-4-yl | 72 | TOF MS ES⁺ exact mass |
| | | | calculated for C₂₄H₂₃N₅O(MH+): |
| | | | 398.1981, found: 398.1955 |
| | | | m.p. 210° C. |
| 21 | (morpholin-4-yl)methyl | 77 | TOF MS ES⁺ exact mass |
| | | | calculated for C₂₅H₂₅N₅O(MH+): |
| | | | 412.2137, found: 412.2120 |
| | | | m.p. 128° C. |
| 22 | 2-(pyrolidin-1-yl)ethoxy | 73 | TOF MS ES⁺ exact mass |
| | | | calculated for C₂₆H₂₇N₅O(MH+): |
| | | | 426.2294, found: 426.2254 |
| | | | m.p. 96° C. |
| 23 | (morpholin-4- | 75 | TOF MS ES⁺ exact mass |
| | yl)carbonylmethoxy | | calculated for C₂₆H₂₅N₅O₃(MH+): |
| | | | 456.2036, found: 456.2012 |
| | | | m.p. 170° C. |

### Triazole Examples

### Example 24

### 2-(4-Methanesulfonylphenyl)-4-(5-(6-methyl)-pyridin-2-yl-3H-[1,2,3]triazol-4-yl)-pyridine

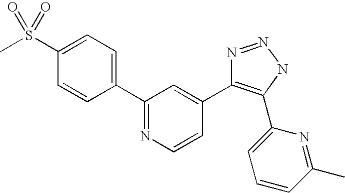

To a solution of intermediate 5 (700 mg, 2 mmol) in dry DMF (13 ml) was added azidotrimethylsilane (8 mmol, 930 mg) and the reaction mixture was stirred at 100° C. overnight. The reaction mixture was hydrolysed with water and extracted with CH₂Cl₂. The organic phase was washed with water, dried over Na₂SO₄ and filtered. Evaporation of the solvent in vacuo gave a crude product which was purified by chromatography on silica gel (toluene/isopropylamine 95:5). The crude oil was precipitated in a mixture CH₂Cl_{2/}hexane to give the title compound as a yellow powder (260 mg, 33.2%), gummy at 150° C.; ¹H NMR (300 MHz, CDCl₃) δ: 8.70 (1H, d), 8.28 (1H, s), 8.15 (2H, d), 7.95 (2H, d), 7.70-7.57 (2H, m), 7.50 (1H, d), 7.15 (1H, d), 3.00 (3H, s), 2.50 (3H, s), NH triazole not observed ; TOF MS ES⁺ exact mass calculated for C₂₀H₁₇N₅O₂S: 392.1181(MH+). Found: 392.1218(MH+) .

The following compounds of formula (IBa) were prepared by methods analogous to that described for Example 24 using the starting materials indicated (see Table 11).

TABLE 11

| | |
|---|---|
| | (IBa) |
| 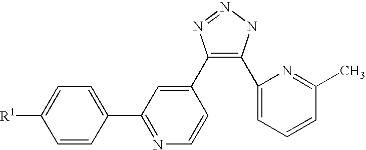 | |

| | | From | |
|---|---|---|---|
| Ex | R¹ | Int. | Physical data |
| 25 | methoxy | 6 | TOF MS ES⁺ exact mass calculated for |
| | | | C₂₀H₁₇N₅O (MH+)/344.1511. |
| | | | Found: 344.1506. |
| 26 | 2-(N,N- | 16 | TOF MS ES⁺ exact mass calculated for |
| | dimethylamino)- | | C₂₃H₂₄N₆O (MH+): 401.2090. |
| | ethoxy | | Found: 401.2063. m.p. 143° C. |
| 27 | morpholinomethyl | 15 | TOF MS ES⁺ exact mass calculated for |
| | | | C₂₄H₂₄N₆O (MH+): 413.2090. |
| | | | Found: 413.2110. m.p. 110° C. |
| 28 | ethyl | 9 | [APCI MS] m/z 342 (MH+) |
| | | | TOF MS ES⁺ exact mass calculated for |
| | | | C₂₁H₁₉N₅ (MH+): 342.1718. |
| | | | Found: 342.1716. |
| 29 | tetrahydropyran-4- | 7 | TOF MS ES⁺ exact mass calculated for |
| | ylaminocarbonyl | | C₂₅H₂₄N₆O (MH+): 441.2039. |
| | | | Found: 441.2032. |
| 30 | chloro | 10 | TOF MS ES⁺ exact mass calculated for |
| | | | C₁₉H₁₄ClN₅ (MH+): 348.1016 |
| | | | Found: 348.1000; m.p. 144° C. |
| 31 | trifluoromethoxy | 11 | TOF MS ES⁺ exact mass calculated for |
| | | | C₂₀H₁₄F₃N₅O (MH+): 398.1229. |
| | | | Found: 398.1194; m.p. 128° C. |
| 32 | 2-(pyrolidin-1- | 12 | TOF MS ES⁺ exact mass calculated for |
| | yl)ethoxy | | C₂₅H₂₆N₆O (MH+): 427.2246. |
| | | | Found: 427.2277. |
| 33 | fluoro | 13 | TOF MS ES⁺ exact mass calculated for |
| | | | C₁₉H₁₄FN₅ (MH+): 332.1311. |
| | | | Found: 332.1345. |

### Imidazopydidine Examples

### Example 34

### 3-[2-(4-methoxyphenyl)-pyridin-4-yl]-2-pyridin-2-yl-imidazo[1,2-a]pyridine

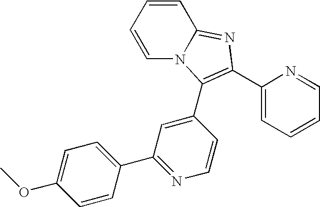

A solution of intermediate 84 (500 mg, 1.42 mmol) in toluene (10 ml) was treated with tetrakis(triphenylphosphine)palladium(0) (165 mg, 10% mol) and stirred at room temperature for 30 min. Na₂CO₃ (2M) (0.6 ml) was added to the reaction mixture, followed by 4-methoxyphenyl boronic acid (282 mg, 1.3 eq, 1.85 mmol). The resulting was heated under reflux overnight. The cooled mixture was poured into ice and reacted with toluene. The organic layer was washed with water, dried over Na₂SO₄ and filtered. Evaporation of the solvent in vacuo gave a crude oil which was purified by chromatography on silica gel (CH₂Cl₂/MeOH, 90/10) and triturated in CH₂Cl₂/pentane to give the title compound as a cream powder (68 mg, 13%); m.p. 222° C.; TOF MS ES⁺ exact mass calculated for C₂₄H₁₈N₄O: 379.1559(MH+). Found: 379.1540 (MH+).

The following compounds of formula (ICj) were prepared by methods analogous to that described for Example 34 using the starting materials indicated (see Table 12).

TABLE 12

| | |
|---|---|
| | (ICj) |
| 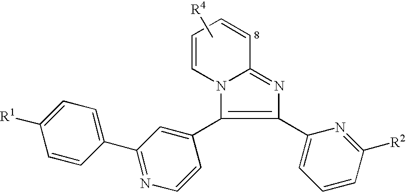 | |

| Ex | R¹ | R⁴ | R² | From Int. | Physical data |
|---|---|---|---|---|---|
| 35 | methoxy | H | methyl | 85 | [APCI MS] m/z: 393 (MH+) m.p. 174° C. |
| 36 | trifluoromethoxy | H | methyl | 85 | [APCI MS] m/z: 447 (MH+) m.p. 120° C. |
| 37 | cyano | H | methyl | 85 | [APCI MS] m/z: 388 (MH+) m.p. 214° C. |
| 38 | methanesulfonyl | H | H | 84 | [APCI MS] m/z: 427 (MH+) m.p. 242-244° C. |
| 39 | methanesulfonyl | H | methyl | 85 | [APCI MS] m/z: 441 (MH+) |
| | | | | | ¹H NMR (300 MHz, CDCl₃, ppm) |
| | | | | | δ: 8.85 (d, 1H), 8.2 (d, 1H), 8.14 (d, 1H), |
| | | | | | 8.09 (m, 2H), 7.82 (d, 1H), 7.74 (d, 1H), |
| | | | | | 7.58 (m, 2H), 7.53 (m, 1H), 7.44 (dd, 1H), |
| | | | | | 7.3 (t, 1H), 7.05 (d, 1H), 6.85 (t, 1H), |
| | | | | | 3.09 (s, 3H), 2.31 (s, 3H) |
| 40 | methanesulfonyl | 8-methyl | methyl | 90 | [APCI MS] m/z: 455 (MH+) |
| | | | | | ¹H NMR (300 MHz, CDCl₃, ppm) |
| | | | | | δ: 8.85 (d, 1H), 8.2 (d, 2H), 8.05 (m, 3H), |
| | | | | | 7.85 (d, 1H), 7.5 (m, 3H), 7.1 (m, 2H), 6.8 |
| | | | | | (t, 1H), 3.1 (s, 3H), 2.75 (s, 3H), 2.35 (s, 3H) |
| 41 | acetyl | H | H | 84 | [APCI MS] m/z: 391 (MH+) |
| | | | | | m.p. 214° C. |
| 42 | methylcarbonyl | H | H | 84 | [APCI MS] m/z: 406 (MH+) |
| | amino | | | | m.p. 133° C. |
| 43 | methylcarbonyl | H | methyl | 85 | TOF MS ES⁺ exact mass calculated for |
| | amino | | | | C₂₆H₂₁N₅O (MH+): 420.1824. |
| | | | | | Found: 420.1808. m.p. 257° C. |
| 44 | (tetrahydropyran- | H | H | 84 + 33 | [APCI MS] m/z: 476 (MH+) |
| | 4-yl)amino | | | | m.p. 179° C. |
| | carbonyl | | | | |
| 45 | (tetrahydropyran- | H | methyl | 85 + 33 | [APCI MS] m/z: 490 (MH+) |
| | 4-yl)amino | | | | m.p. 128° C. |
| | carbonyl | | | | |
| 47 | (morpholin-4- | 8-methyl | methyl | 90 + 35 | [APCI MS] m/z: 490 (MH+) |
| | yl)carbonyl | | | | |
| 48 | (4-ethylpiperazin- | 8-methyl | methyl | 90 + 36 | TOF MS ES⁺ exact mass calculated for |
| | 1-yl)carbonyl | | | | C₃₂H₃₂N₆O (MH+): 517.2715, |
| | | | | | found: 517.2751 m.p. 212° C. |
| 49 | (morpholin-4- | H | fluoro | 92 + 35 | TOF MS ES⁺ exact mass calculated for |
| | yl)carbonyl | | | | C₂₈H₂₂FN₅O₂ (MH+): 480.1836, |
| | | | | | found: 480.1756 m.p. 191° C. |

The following compounds of formula (ICk) were prepared by methods analogous to that described for Example 34 using the starting materials indicated (see Table 13).

TABLE 13

| | |
|---|---|
| | (ICk |
| 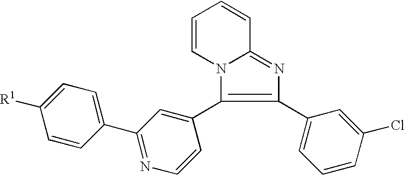 | |

| | | From | |
|---|---|---|---|
| Ex. | R¹ | Int. | Physical data |
| 50 | methanesulfonyl | 91 + 34 | TOF MS ES⁺ exact mass calculated for |
| | amino | | C₂₅H₁₉ClN₄O₂S (MH+): 475.0995, |
| | | | found: 475.0975; m.p. 60° C. (becomes |
| | | | gummy) |
| 51 | (tetrahydropyran- | 91 + 33 | ¹H NMR (300 MHz, CDCl₃, ppm) δ: |
| | 4-yl)amino | | 7.85 (d, 2H), 7.75 (d, 2H), 7.65-7.55 |
| | carbonyl | | (m, 4H), 7.45-7.35 (m, 6H), 7.2 (m, |
| | | | 1H), 6 (d, 1H), 4.2 (m, 1H), 4 (m, 2H), |
| | | | 3.5 (m, 2H), 2, (m, 2H), 1.6 (m, 2H); |
| | | | m.p. 234° C. |

### Example 52

### 2-(6-methyl-pyridin-2-yl)-3-{2-[4-amino-phenyl]-Pyridin-4-yl}-imidazo[1,2-a]pyridine

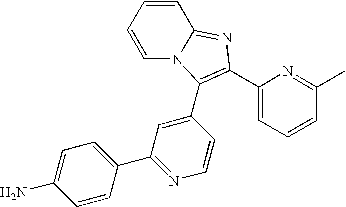

A solution of example 43 (2.3 g, 5.48 mmol) in MeOH (50 ml) and HCl 1N (50 ml) was stirred at room temperature for 18 hours and then basified with a solution of NaOH 1N. After extraction with CH₂Cl₂, the organic phase was dried over Na₂SO₄ and concentrated under reduced pressure. The title compound was obtained as a yellow solid (0.79 g, 38%); [APCl MS] m/z: 378 (MH⁺); ¹H NMR (300 MHz, CDCl₃) δ ppm: 8.7 (d, 1H), 8.1 (d, 1H), 7.85 (m, 3H), 7.7 (d, 1H), 7.6 (d, 1H), 7.45 (t, 1H), 7.25 (m, 2H), 7 (d, 1H), 6.8 (t, 1H), 6.7 (d, 2H), 3.85 (m, 2H), 2.4 (s, 3H).

### Example 53

### 2-(6-methyl-pyridin-2-yl)-3-{2-[4-(trifluoromethylsulfonylamino)phenyl]-pyridin-4-yl}-imidazo[1,2-a]pyridine

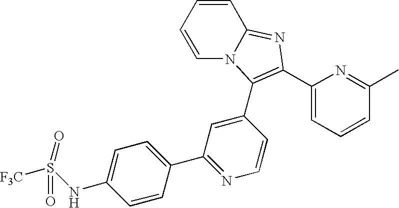

To a solution of example 52 (390 mg, 1.03 mmol) in CH₂Cl₂ (10 ml) were added trifluoromethanesulfonic anhydride (0.2 ml, 8.55 mmol) and triethylamine (0.17 ml, 1.24 mmol) and the mixture was stirred at room temperature for 3 days and then poured into water. After extraction with CH₂Cl₂, the organic phase was dried over Na₂SO₄ and concentrated under reduced pressure. The residue was purified by chromatography on silica gel, eluting with CH₂Cl₂/MeOH (9/1). The title compound was obtained as a yellow foam (178 mg, 33.8%); m.p. 135° C.; TOF MS ES⁺ exact mass calculated for C₂₅H₁₈F₃N₅O₂S: 510.1212(MH+). Found: 510.1229(MH+).

### Example 54

### 3-[2-(4-(morpholin-4-yl)-phenyl)-pyridin-4-yl]-2-pyridin-2-yl-imidazo[1,2-a]pyridine

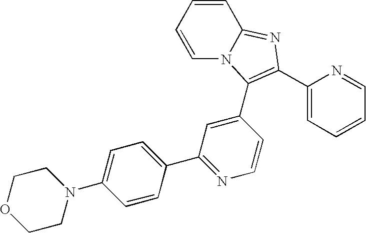

A mixture of intermediate 112 (400 mg, 0.93 mmol), morpholine (1.2 eq, 0.1 ml, 1.1 mmol); Pd₂(dba)₃ (0.05 eq, 43 mg, 0.05 mmol), BINAP (0.15 eq, 88 mg, 0.14 mmol) and potassium tert-butoxide (1.4 eq, 126 mg, 1.31 mmol) in toluene (50 ml) was heated under reflux for 2 hours. After dilution with CH₂Cl₂, the organic phase was washed with water and dried (Na₂SO₄). The solvent was removed under reduced pressure and the resulting residue purified by chromatography on silica gel eluting with CH₂Cl₂/MeOH (98:2, 95:5 and then 93:7). The resulting oil was crystallised from CH₂Cl₂/pentane to give the title compound as a yellow solid (140 mg, 35%); m.p. 145° C. (becomes gummy); TOF MS ES⁺ exact mass calculated for C₂₇H₂₃N₅O: 434.1981 (MH+). Found: 434.1993(MH+).

### Example 55

### 3-{2-[4-(4-methylpiperazin-1-yl)-phenyl]-pyridin-4-yl}-2-pyridin-2-yl-imidazo[1,2-a]pyridine

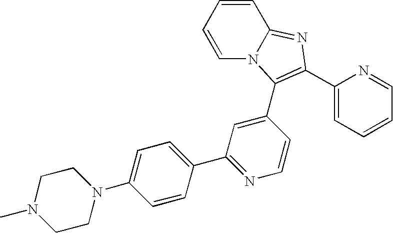

Intermediate 112 (400 mg, 0.94 mmol) and N-methyl-piperazine (0.125 ml, 1.2 eq, 1.13 mmol) were coupled and treated as described for example 54 to afford, after crystallisation in CH₂Cl₂/diisopropyl ether, the title compound as cream crystals (70 mg, 17%); m.p. 150° C. (become gummy); [APCl MS] m/z 447 (MH+).

### Example 56

### 2-(6-methyl-pyridin-2-yl)-3-[2-(4-(morpholin-4-ylmethyl)phenyl)-pyridin-4-yl]-imidazo[1,2-a]pyridine

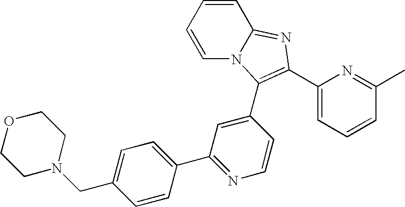

To a solution of intermediate 99 (310 mg, 0.79 mmol) and morpholine (1.5 eq, 0.1 ml, 1.2 mmol) in dry dichloroethane (30 ml) was added sodium triacetoxyborohydride (1.5 eq, 253 mg, 1.2 mmol) and the mixture was stirred for 3 hours at room temperature. The mixture was basified with NaOH 1N, the aqueous layer was extracted with CH₂Cl₂ and dried over Na₂SO₄. The resulting product was recrystallised from ethyl acetate to give the title compound as a white powder (194 mg, 53%); m.p. 156° C.; [APCl MS] m/z 462 (MH+).

The following compounds of formula (ICj) were prepared by methods analogous to that described for Example 56 using the starting materials indicated (see Table 14).

TABLE 14

| | |
|---|---|
| | (ICj) |
| 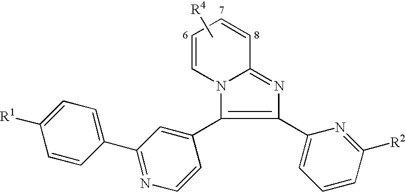 | |

| Ex. | R¹ | R⁴ | R² | From Int. | Physical data |
|---|---|---|---|---|---|
| 57 | (morpholin-4-yl)- | H | H | 100 | [APCI MS] m/z 448 (MH+) |
| | methyl | | | | m.p. 80° C. (degradation) |
| 58 | (morpholin-4-yl)- | 6-chloro | methyl | 103 | [APCI MS] m/z 496 (MH+) |
| | methyl | | | | m.p. 157° C. |
| 59 | (morpholin-4-yl)- | 7-methyl | methyl | 104 | TOF MS ES⁺ exact mass calculated |
| | methyl | | | | for C₃₀H₂₉N₅O (MH+): 476.2450. |
| | | | | | Found: 476.2445; m.p. 188° C. |
| 60 | (pyrolidin-1-yl)- | 6-chloro | methyl | 103 | TOF MS ES⁺ exact mass calculated |
| | methyl | | | | for C₂₉H₂₆ClN₅ (MH+): 480.1955. |
| | | | | | Found: 480.1900; m.p. 134° C. |
| 61 | (morpholin-4-yl)- | 8-methyl | methyl | 105 | [APCI MS] m/z 476 (MH+) |
| | methyl | | | | m.p. 122° C. |
| 62 | (pyrolidin-1-yl)- | 8-methyl | methyl | 105 | 1H NMR (300 MHz, DMSO d6, ppm) |
| | methyl | | | | δ: 8.95 (d, 1H), 8.45 (m, 2H), 8.2 (d, |
| | | | | | 2H), 7.95 (t, 1H), 7.8 (d, 2H), 7.75 (m, |
| | | | | | 3H), 7.5 (d, 1H), 7.25 (t, 1H), 4.4 (m, |
| | | | | | 2H), 3.3 (m, 2H), 3.05 (m, 2H), 2.95 |
| | | | | | (s, 3H), 2.5 (s, 3H), 2.05 (m, 2H), 1.9 |
| | | | | | (m, 2H); m.p: 197° C. |

The following compounds of formula (ICm) were prepared by methods analogous to that described for Example 56 using the starting materials indicated (see Table 15).

TABLE 15

| | |
|---|---|
| | (ICm) |
| 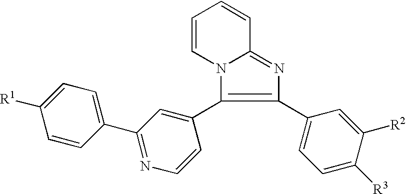 | |

| Ex. | R¹ | R² | R³ | From Int. | Physical data |
|---|---|---|---|---|---|
| 63 | (morpholin-4-yl)- | chloro | fluoro | 101 | [APCI MS] m/z 499 (MH+) |
| | methyl | | | | m.p. 189° C. |
| 64 | (pyrolidin-1-yl)- | fluoro | fluoro | 102 | TOF MS ES⁺ exact mass calculated |
| | methyl | | | | for C₂₉H₂₄F₂N₄ (MH+): 467.2047. |
| | | | | | Found: 467.2063 m.p. 155° C. |
| 65 | (morpholin-4-yl)- | fluoro | fluoro | 102 | TOF MS ES⁺ exact mass calculated |
| | methyl | | | | for C₂₉H₂₄F₂N₄O (MH+): 483.1996. |
| | | | | | Found: 483.2030 m.p. 205° C. |

### Example 66

### 2-(6-methyl-pyridin-2-yl)-3-{2-[4-((morpholin-4-yl)carbonyl)phenyl]-pyridin-4-yl}-imidazo[1,2-a]pyridine

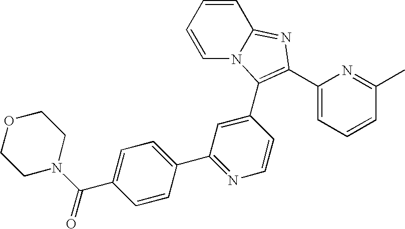

To a solution of intermediate 106 (500 mg, 1.23 mmol) in DMF (30 ml) were added morpholine (0.13 ml, 1.48 mmol), HOBT (200 mg, 1.48 mmol), EDCl (283 mg, 1.48 mmol) and triethylamine (0.2 ml; 1.48 mmol) and the mixture was stirred at room temperature overnight and then diluted with CH₂Cl₂. The organic phase was washed with sodium hydroxide solution 1N, then water, dried over Na₂SO₄, and concentrated under reduced pressure. The residue was purified by chromatography on silica gel, eluting with CH₂Cl₂/MeOH (95:5). After trituration with diisopropyl ether, the title compound was obtained as a pale yellow solid (147 mg, 25.13%); m.p. 110° C.; [APCl MS] m/z 476 (MH⁺).

### Example 67

### 2-(6-methyl-pyridin-2-yl)-3-{2-[4-((3-methoxypropylamino)carbonyl)phenyl]-pyridin-4-yl}-imidazo[1,2-a]pyridine

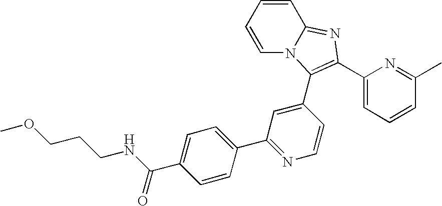

Intermediate 106 (400 mg, 0.98 mmol) and 3-methoxypropylamine (0.11 ml, 1.18 mmol) were coupled and treated as described for example 66 to afford, after trituration with CH₂Cl₂/pentane, the title compound as a pale yellow solid (210 mg, 44.69%); m.p. 165° C.; [APCl MS] m/z 478 (MH⁺).

### Example 68

### 2-(pyridin-2-yl)-3-{2-[4-(2-(pyrrolidin-1-yl)ethoxy)phenyl]-pyridin-4-yl}-imidazo[1,2-a]pyridine

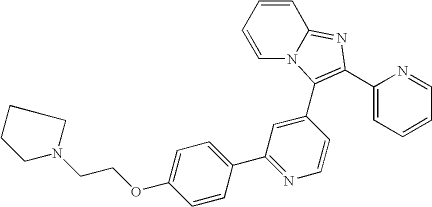

A solution of intermediate 113 (140 mg, 0.3 mmol) and pyrrolidine (0.75 ml, 9 mmol) in EtOH (5 ml) was heated under reflux for 6 days. After cooling, water was added and the product was extracted with CH₂Cl₂. The organic phase was dried over Na₂SO₄, filtered and the solvent was removed under reduced pressure. The resulting residue was purified by chromatography on silica gel eluting with CH₂Cl₂/MeOH/TEA (80/20/1%). The title compound was obtained as a brown gum (13 mg, 10%); [APCl MS] m/z 462 (MH+); ¹H NMR (300 MHz, CDCl₃) δppm: 8.75 (d, 1H), 8.5 (d, 1H), 8.15 (d, 1H), 7.9 (m, 3H), 7.85 (s, 1H), 7.7 (m, 2H), 7.3 (m, 2H), 7.2 (m, 1H), 7 (d, 2H), 6.85 (t, 1H), 4.2 (t, 2H), 3 (t, 2H), 2.75 (m, 4H), 1.85 (m, 4H).

The following compounds of formula (ICp) were prepared by methods analogous to that described for Example 68 using the starting materials indicated (see Table 16).

TABLE 16

(ICp)

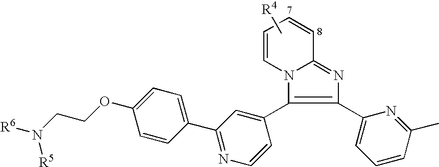

| | | | From | |
|---|---|---|---|---|
| Ex. | R⁵R⁶N | R⁴ | Int. | Physical data |
| 69 | dimethyl | H | 114 | [APCI MS] m/z: 450 (MH+) |
| | amino | | | ¹H NMR (300 MHz, CDCl₃, ppm) δ: |
| | | | | 8.8 (d, 1H), 8.2 (d, 1H), 7.95 (m, |
| | | | | 3H), 7.75 (m, 2H), 7.6 (t, 1H), 7.4 |
| | | | | (d, 1H), 7.3 (m, 1H), 7.05 (m, 3H), |
| | | | | 6.9 (t, 1H), 4.25 (t, 2H), 3 (t, 2H), |
| | | | | 2.55 (s, 6H), 2.4 (s, 3H) |
| 70 | pyrolidin- | 7- | 116 | TOF MS ES⁺ exact mass calculated |
| | 1-yl | methyl | | for C₃₁H₃₁N₅O (MH+): 490.2607. |
| | | | | Found: 490.2600; m.p. 163° C. |
| 71 | morpholin- | 7- | 116 | TOF MS ES⁺ exact mass calculated |
| | 4-yl | methyl | | for C₃₁H₃₁N₅O₂ (MH+): 506.2556. |
| | | | | Found: 506.2534; ¹H NMR (300 |
| | | | | MHz, CDCl₃, ppm) δ: 8.8 (d, 1H), |
| | | | | 8.05 (d, 1H), 7.9 (m, 3H), 7.7 (d, |
| | | | | 1H), 7.6 (t, 1H), 7.5 (s, 1H), 7.35 (d, |
| | | | | 1H), 7 (m, 3H), 6.7 (d, 1H), 4.2 (t, |
| | | | | 2H), 3.8 (m, 4H), 2.85 (t, 2H), 2.6 |
| | | | | (m, 4H), 2.45 (s, 3H), 2.4 (s, 3H) |
| 72 | pyrolidin- | 8- | 117 | [APCI MS] m/z: 490 (MH+); |
| | 1-yl | methyl | | ¹H NMR (300 MHz, CDCl₃, ppm) δ: |
| | | | | 8.75 (d, 1H), 8.05 (d, 1H), 7.9 (m, |
| | | | | 3H), 7.7 (d, 1H), 7.55 (t, 1H), 7.3 (d, |
| | | | | 1H), 7.05 (m, 2H), 6.95 (d, 2H), 6.7 |
| | | | | (t, 1H), 4.25 (t, 2H), 3.05 (t, 2H), |
| | | | | 2.9 (m, 4H), 2.7 (s, 3H), 2.4 (s, 3H), |
| | | | | 1.95 (m, 4H) |

The following compounds of formula (ICq) were prepared by methods analogous to that described for Example 68 using the starting materials indicated (see Table 17).

TABLE 17

(ICq)

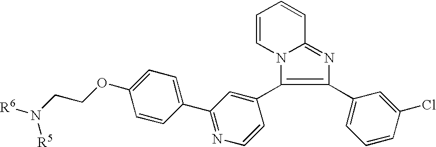

| | | From | |
|---|---|---|---|
| Ex. | R⁶R⁵N | Int. | Physical data |
| 73 | dimethylamino | 115 | TOF MS ES⁺ exact mass calculated for |
| | | | C₂₈H₂₅ClN₄O (MH+): 469.1795. Found: |
| | | | 469.1723; ¹H NMR (300 MHz, CDCl₃, ppm) |
| | | | δ: 8.8 (d, 1H), 8.1 (d, 1H), 7.9 (d, 2H), 7.8 |
| | | | (s, 1H), 7.7 (m, 2H), 7.45 (d, 1H), 7.25 (m, |
| | | | 3H), 7.2 (m, 1H), 7 (d, 2H), 6.85 (t, 1H), |
| | | | 4.35 (t, 2H), 3.25 (t, 2H), 2.7 (s, 6H) |
| 74 | pyrolidin-1-yl | 115 | TOF MS ES⁺ exact mass calculated for |
| | | | C₃₀H₂₇ClN₄O (MH+): 495.1952. Found: |
| | | | 495.1957; m.p. 298° C. |

### Example 75

### 7-methyl-2-(6-methyl-pyridin-2-yl)-3-{2-[4-((1-methyl-imidazol-4-yl)methyloxy)phenyl]-pyridin-4-yl}-imidazol[1,2-a]pyridine

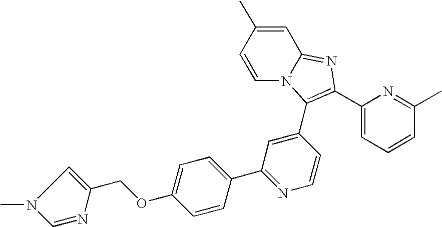

To a solution of intermediate 111 (400 mg, 1.02 mmol) in DMF (20 ml) was added portionwise sodium hydride (60% in mineral oil, 101 mg, 2.55 mmol) and the mixture was stirred at room temperature for 20 minutes. Intermediate 22 (173 mg, 1.32 mmol) was then added and the mixture was heated at 60° C. for 3 days and then poured into water. After extraction with CH₂Cl₂, the organic phase was dried over Na₂SO₄ and concentrated under reduced pressure. The residue was purified by chromatography on silica gel eluting with CH₂Cl₂/MeOH (90:10). After trituration with diisopropyl oxide, the title compound was obtained as a yellow solid (130 mg, 26%); m.p. 217° C.; TOF MS ES⁺ exact mass calculated for C₃₀H₂₆N₆O: 487.2246(MH+). Found: 487.2247(MH+).

### Example 76

### 2-(6-methyl-pyridin-2-yl)-3-{2-[4-((1-methyl-imidazol-4-yl)methyloxy)phenyl]-pyridin-4-yl}-imidazo[1,2-a]pyridine

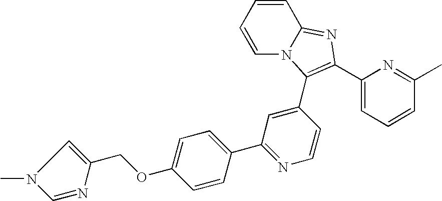

Intermediate 108 (400 mg, 1.06 mmol) and intermediate 22 (212 mg, 1.27 mmol) were coupled and treated as described for example 75 to afford, after trituration with diisopropyl oxide, the title compound as a white solid (200 mg, 40%); m.p. 120° C.; [APCl MS] m/z 473 (MH+).

### Example 77

### 7-methyl-2-(6-methyl-pyridin-2-yl)-3-{2-[4(aminocarbonylmethyloxy)phenyl]-pyridin-4-yl}-imidazo[1,2-a]pyridine

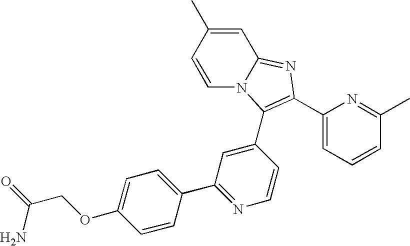

To a solution of intermediate 111 (500 mg, 1.27 mmol) in acetone (25 ml) were added cesium carbonate (623 mg, 1.91 mmol) and bromoacetamide (264 mg, 1.91 mmol) and the mixture was heated under reflux for 48 hours and then poured into water. After extraction with CH₂Cl₂, the organic phase was dried over Na₂SO₄ and concentrated under reduced pressure. The residue was purified by chromatography on silica gel eluting with CH₂Cl₂/MeOH (95/5). After trituration with pentane/ethyl acetate, the title compound was obtained as a pale yellow solid (133 mg, 23%); m.p. 213° C.; (APCl MS] m/z 450 (MH⁺).

### Example 78

### 8-methyl-2-(6-methyl-pyridin-2-yl)-3-{2-[4-(aminocarbonylmethyloxyl)phenyl]-pyridin-4-yl}-imidazo[1,2-a]pyridine

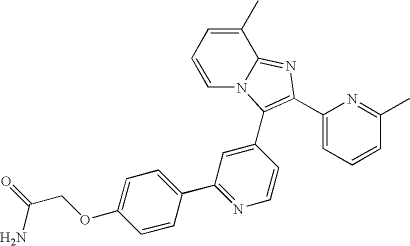

Intermediate 110 (500 mg, 1.27 mmol) and bromoacetamide (264 mg, 1.91 mmol) were coupled and treated as described for example 77 to afford, after trituration with diisopropyl oxide, the title compound as a cream solid (80 mg, 14%); m.p. 183° C.; [APCl MS] m/z 450 (MH+).

### Example 79

### 2-(6-methyl-pyridin-2-yl)-3-{2-[4-(morpholin-4-yl)phenyl]-pyridin-4yl}-imidazo[1,2-a]pyridine

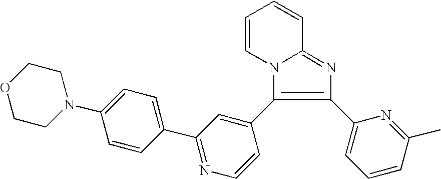

To a solution of intermediate 48 (3 g, 8.04 mmol) in CH₂Cl₂ (80 ml) was added bromine-polymer-supported (5.03 g, 8.04 mmol) and the suspension was stirred at room temperature for 3 hours. The resin was removed by filtration, with the filtrate being added directly to 2-amino-pyridine (1.51 g, 16.08 mmol) and the resin washed many times with ethanol. The filtrate was heated at reflux for 18 hours, allowed to cool and concentrated. The residue was treated with water and extracted with CH₂Cl₂. The organic phase was dried over Na₂SO₄, filtered, and concentrated under reduced pressure. The residue was purified by chromatography on silica gel (CH₂Cl₂/MeOH, 98/2 then 95:5) to give an oil which crystallised by trituration from diisopropyl oxide. The title compound was obtained as cream crystals (1.2 g; 33.38%); m.p. 190° C.; TOF MS ES⁺ exact mass calculated for C₂₈H₂₅N₅O (MH+): 448.2137. Found: 448.2081

### Example 80

### 7-methyl-2-(6-methyl-pyridin-2-yl)-3-{2-[4-(morpholin-4-yl)phenyl]-pyridin-4-yl}-imidazo[1,2-a]pyridine

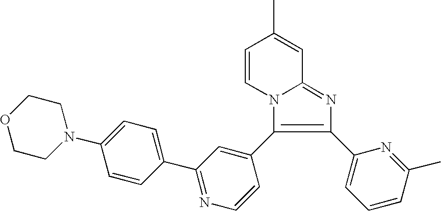

Intermediate 48 (1.27 g, 3.4 mmol) was reacted as described for example 79, to afford after trituration with diisopropyl oxide, the title compound as cream crystals (0.6 g, 38.22%); m.p. 208° C.; TOF MS ES⁺ exact mass calculated for C₂₉H₂₇N₅O (MH+): 462.2294. Found: 462.2263

### Imidazole Examples

### Example 81

### N-(tetrahydropyran--4-yl)-(4-(4-{2-tert-Butyl-5-[6-methyl-pyridin-2-yl]-1H-imidazol-4-yl}pyridin-2-yl)-benzamide

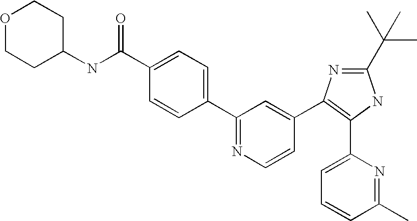

To a solution of intermediate 120 (0.95 g, 2.56 mmol) in a mixture of DME (30 ml) and water (15 ml) were added intermediate 33 (0.93 g, 2.81 mmol), tetrakis(triphenylphosphine) palladium(0) (0.1 g, 0.086 mmol) and Na₂CO₃ (solution 2M, 5 ml) and the mixture was heated under reflux overnight and then poured into water. After extraction with CH₂Cl₂, the organic phase was dried over Na₂SO₄, and concentrated under reduced pressure. The residue was recrystallised from EtOAc to afford the title compound as yellow crystals (0.77 g, 55.36%); m.p. 174° C. ; TOF MS ES⁺ exact mass calculated for C₃₀H₃₃N₅O₂: 496.2712(MH+). Found: 496.2662 (MH+).

The following compounds of formula (IDf) were prepared by methods analogous to that described for Example 81 using the starting materials indicated (see Table 18).

TABLE 18

| | |
|---|---|
| | (IDf) |
| 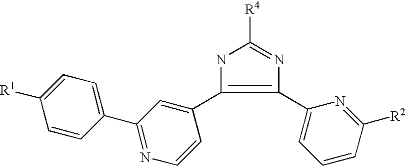 | |

| Ex. | R¹ | R⁴ | R² | From Int. | Physical data |
|---|---|---|---|---|---|
| 82 | (tetrahydro- | t-butyl | H | 121 + 33 | TOF MS ES⁺ exact mass calculated |
| | pyran-4-yl)- | | | | for C₂₉H₃₁N₅O₂ (MH+): 482.2556. |
| | aminocarbonyl | | | | Found 482.2577; m.p. 182° C. |
| 83 | methoxy | t-butyl | H | 121 | TOF MS ES⁺ exact mass calculated |
| | | | | | for C₂₄H₂₄N₄O (MH+): 385.2028. |
| | | | | | Found: 385.2026; m.p. 143° C. |
| 84 | methane- | t-butyl | methyl | 120 | TOF MS ES⁺ exact mass calculated |
| | sulfonyl | | | | for C₂₅H₂₆N₄O₂S (MH+): 447.1855. |
| | | | | | Found: 447.1905; m.p. 144° C. |
| 85 | chloro | t-butyl | methyl | 120 | TOF MS ES⁺ exact mass calculated |
| | | | | | for C₂₄H₂₃ClN₄ (MH+): 403.1689. |
| | | | | | Found: 403.1637; m.p. 122° C. |
| 86 | morpholin-4-yl | t-butyl | methyl | 120 + 27 | TOF MS ES⁺ exact mass calculated |
| | | | | | for C₂₈H₃₁N₅O (MH+): 454.2607. |
| | | | | | Found: 454.2576; m.p. 238° C. |
| 87 | trifluoro- | t-butyl | methyl | 120 | TOF MS ES⁺ exact mass calculated |
| | methoxy | | | | for C₂₅H₂₃F₃N₄O (MH+): 453.1902. |
| | | | | | Found: 453.1863; m.p. 179° C. |
| 88 | (morpholin-4- | t-butyl | methyl | 120 + 35 | TOF MS ES⁺ exact mass calculated |
| | yl) carbonyl | | | | for C₂₉H₃₁N₅O₂ (MH+): 482.2556. |
| | | | | | Found: 482.2517; m.p. 180° C. |
| 89 | (4-ethyl- | t-butyl | methyl | 120 + 36 | TOF MS ES⁺ exact mass calculated |
| | piperazin-1- | | | | for C₃₁H₃₆N₆O (MH+): 509.3029. |
| | yl)carbonyl | | | | Found: 509.3025; ¹H NMR (300 |
| | | | | | MHz; CDCl₃, ppm) δ: 8.53 (1H, d), |
| | | | | | 7.93-7.84 (3H, m), 7.40 (1H, d), |
| | | | | | 7.37-7.27 (3H, m), 7.18 (1H, d), 6.86 |
| | | | | | (1H, d), 3.85-3.23 (4H, brd), 2.53- |
| | | | | | 2.21 (9H, m), 1.31 (9H, s), 0.99 (3H, brs). |
| 90 | morpholin-4-yl | t-butyl | H | 121 + 27 | TOF MS ES⁺ exact mass calculated |
| | | | | | for C₂₇H₂₉N₅O (MH+): 440.2450. |
| | | | | | Found: 440.2401; ¹H NMR (300 |
| | | | | | MHz; CDCl₃, ppm) δ: 9.92 (1H, brs), |
| | | | | | 8.47 (1H, d), 8.38 (1H, d), 7.81 (2H, |
| | | | | | d), 7.77 (1H, s), 7.36 (2H, d), 7.27 |
| | | | | | (1H, d), 6.95 (1H, dd), 6.80 (2H, d), 3.70 |
| | | | | | (4H, brt), 3.06 (4H, brt), 1.30 (9H, s). |
| 91 | 4-ethyl- | t-butyl | H | 121 + 28 | TOF MS ES⁺ exact mass calculated |
| | piperazin-1-yl | | | | for C₂₉H₃₄N₆ (MH+): 467.2923. |
| | | | | | Found: 467.2880; m.p. 190-192° C. |
| 92 | 2-(pyrolidin-1- | t-butyl | H | 121 + 29 | TOF MS ES⁺ exact mass calculated |
| | yl)ethoxy | | | | for C₂₇H₃₃N₅O (MH+): 468.2763. |
| | | | | | Found: 468.2729; ¹H NMR (300 MHz; |
| | | | | | CDCl₃, ppm) δ: 10.02 (1H, brs), |
| | | | | | 8.63 (1H, d), 8.53 (1H, d), 7.94 (2H, d), |
| | | | | | 7.91 (1H, s), 7.53-7.48 (2H, m), 7.43 |
| | | | | | (1H, d), 7.12-7.06 (1H, m), 6.96 (2H, |
| | | | | | d), 4.18 (2H, brt), 2.95 (2H, brt), 2.68 |
| | | | | | (4H, brs), ), 1.82 (4H, brs), 1.45 (9H, s). |
| 93 | 4-ethyl- | t-butyl | methyl | 120 + 28 | TOF MS ES⁺ exact mass calculated |
| | piperazin-1-yl | | | | for C₃₀H₃₆N₆ (MH+): 481.3080. |
| | | | | | Found: 481.3092; m.p. 210° C. |
| 94 | methanesulfonyl | i-propyl | H | 123 | TOF MS ES⁺ exact mass calculated |
| | | | | | for C₂₃H₂₂N₄O₂S (MH+): 419.1542. |
| | | | | | Found: 419.1543; m.p. 134° C. |
| 95 | methane- | i-propyl | methyl | 122 | TOF MS ES⁺ exact mass calculated |
| | sulfonyl | | | | for C₂₄H₂₄N₄O₂S (MH+): 433.1698. |
| | | | | | Found: 433.1654; ¹H NMR (300 MHz; |
| | | | | | CDCl₃, ppm) δ: 8.62 (1H, d), 8.14 |
| | | | | | (2H, d), 8.09 (1H, s), 7.95 (2H, d), 7.52 |
| | | | | | (1H, d), 7.45 (1H, t), 7.31 (1H, d), 6.98 |
| | | | | | (1H, d), 3.16-3.04 (1H, m), 3.02 (3H, |
| | | | | | s), 2.41 (3H, s), 1.29 (6H, d). |
| 96 | (tetrahydropyran- | i-propyl | methyl | 122 + 33 | TOF MS ES⁺ exact mass calculated |
| | 4-yl)amino- | | | | for C₂₉H₃₁N₅O₂ (MH+): 482.2556. |
| | carbonyl | | | | Found: 482.2509; m.p. 233° C. |
| 97 | morpholin-4-yl | i-propyl | methyl | 122 + 27 | TOF MS ES⁺ exact mass calculated |
| | | | | | for C₂₇H₂₉N₅O (MH+): 440.2450. |
| | | | | | Found 440.2419: m.p. 160° C. (becomes |
| | | | | | gummy); ¹H NMR (300 MHz; CDCl₃, |
| | | | | | ppm) δ: 10.52 (1H, brs), 8.58 (1H, d), |
| | | | | | 7.94 (2H, d), 7.90 (1H, s), 7.45-7.30 |
| | | | | | (3H, m), 7.00-6.87 (3H, m), 6.80 (2H, |
| | | | | | d), 3.83 (4H, brt), 3.20 (4H, brt), 3.17- |
| | | | | | 3.06 (1H, m), 2.47 (3H, s), 1.35 (6H, d). |
| 98 | morpholin-4-yl | methyl | methyl | 124 + 27 | TOF MS ES⁺ exact mass calculated |
| | | | | | for C₂₅H₂₅N₅O (MH+): 412.2137. |
| | | | | | Found: 412.2155; ¹H NMR (300 MHz; |
| | | | | | CDCl₃, ppm) δ: 8.65 (1H, d), 8.03 |
| | | | | | (1H, s), 7.97 (2H, d), 7.55-7.40 |
| | | | | | (3H, m), 7.04 (1H, d), 6.97 (2H, d), 3.89 |
| | | | | | (4H, brt), 3.26 (4H, brt), 2.59 (3H, s), |
| | | | | | 2.56 (3H, s), NH imidazole not seen |
| 99 | (morpholin-4- | methyl | methyl | 124 + 35 | TOF MS ES⁺ exact mass calculated |
| | yl)carbonyl | | | | for C₂₆H₂₅N₅O₂ (MH+): 440.2086. |
| | | | | | Found: 440.2144; ¹H NMR (300 MHz; |
| | | | | | CDCl₃, ppm) δ: 8.72 (1H, d), |
| | | | | | 8.14 (1H, s), 8.09 (2H, d), 7.63- |
| | | | | | 7.41 (5H, m), 7.08 (1H, d), 3.94-3.62 |
| | | | | | (8H, m), 2.62 (3H, s), 2.60 (3H, s), |
| | | | | | NH imidazole not seen. |
| 100 | morpholin-4-yl | 1-propyl | H | 123 + 27 | TOF MS ES⁺ exact mass calculated |
| | | | | | for C₂₆H₂₇N₅O (M + 1)+: 425.2294. |
| | | | | | Found: 425.2295; m.p. 16° C. (becomes |
| | | | | | gummy); ¹H NMR (300 MHz; CDCl₃, |
| | | | | | ppm) δ: 8.57 (1H, d), 8.47 (1H, d), 7.92 |
| | | | | | (1H, s), 7.87 (2H, d), 7.49 (2H, brd), |
| | | | | | 7.36 (1H, d), 7.15-7.02 (1H, m), |
| | | | | | 6.90 (2H, d), 3.80 (4H, brt), 3.16 (4H, |
| | | | | | brt), 3.14-3.03 (1H, m), |
| | | | | | 1.34 (6H, d), NH imidazole not seen. |

### Example 101

### (4-{4-[2-tert-Butyl-5-(6-methyl-pyridin-2-yl)-1H-imidazol-4-yl]-pyridin-2-yl}-benzyl)-dimethyl-amine

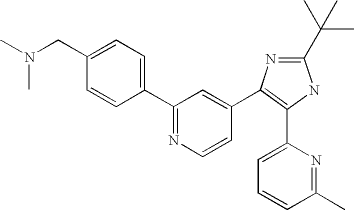

To a solution of intermediate 125 (0.8 g, 2.02 mmol) in CH₂Cl₂ (50 ml) were added dimethylamine (2M solution in MeOH, 1.1 ml, 2.22 mmol) and sodium triacetoxyborohydride (0.8569, 4.04 mmol) and the mixture was stirred at room temperature for 3 hours. The reaction mixture was poured into a saturated solution of NaHCO₃ and extracted with CH₂Cl₂ . The organic phase was dried over Na₂SO₄, and concentrated under reduced pressure. The title compound was obtained, after chromatography on silica gel (CH₂Cl₂/MeOH 98:2 then 85:15) and recrystallisation from EtOAc, as a white solid (0.109 g, 12.6%); m.p. 187° C.; TOF MS ES⁺ exact mass calculated for C₂₇H₃₁N₅:426.2657(MH+). Found: 426.2680(MH+).

The following compounds of formula (IDg) were prepared by methods analogous to that described for Example 101 using the starting materials indicated (see Table 19).

TABLE 19

(IDg)

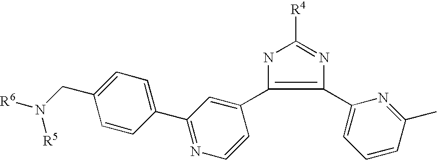

| | | | From | |
|---|---|---|---|---|
| Ex. | R¹ | R⁴ | Int: | Physical data |
| 102 | morpholin- | t-butyl | 125 | TOF MS ES⁺ exact mass |
| | 4-yl | | | calculated for C₂₉H₃₃N₅O (MH+): |
| | | | | 468.2763. Found: 468.2764; |
| | | | | ¹H NMR (300 MHz; CDCl₃, ppm) |
| | | | | δ: 8.77 (1H, d), 8.13 (1H, s), 8.05 |
| | | | | (2H, d), 7.65-7.41 (5H, m), 7.09 |
| | | | | (1H, d), 3.88-3.78 (4H, m), 3.69- |
| | | | | 3.62 (2H, m), 2.64 (3H, s), 2.60- |
| | | | | 2.54 (4H, m), 1.56 (9H, s), |
| | | | | NH imidazole not seen. |
| 103 | pyrolidin- | i-butyl | 125 | TOF MS ES⁺ exact mass |
| | 1-yl | | | calculated for C₂₉H₃₃N₅ (MH+): |
| | | | | 452.2814. Found: 452.2814; |
| | | | | m.p. 148° C. |
| 104 | morpholin- | i-propyl | 127 | TOF MS ES⁺ exact mass |
| | 4-yl | | | calculated for C₂₈H₃₁N₅O (MH+): |
| | | | | 454.2607. Found: 454.2574; |
| | | | | m.p. 141° C. |
| 105 | dimethyl- | i-propyl | 127 | TOF MS ES⁺ exact mass |
| | amino | | | calculated for C₂₆H₂₉N₅ (MH+): |
| | | | | 412.2501. Found: 412.2523; |
| | | | | m.p. 135° C. |

### Example 106

### 4-(2-tert-Butyl-5-{6-methyl}-pyridin-2-yl-1H-imidazol-4-yl)-2-[4-(1-methyl-1H-imidazol-4-ylmethoxy)-phenyl-pyridine

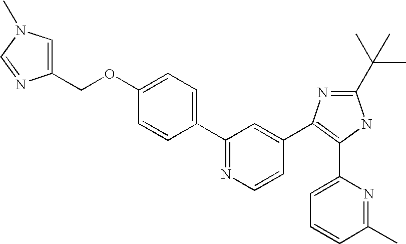

To a solution of intermediate 126 (0.49 g, 1.27 mmol) in DMF (20 ml) was added portionwise sodium hydride (60% in mineral oil, 0.152 g, 3.81 mmol) and the mixture was stirred at room temperature for 10 minutes. Intermediate 22 (0.3 g, 1.8 mmol) was then added and the mixture was stirred for 18 hours at room temperature and then poured into water. After extraction with EtOAc, the organic phase was washed with a solution of NaOH (1N) and water, dried over Na₂SO₄ and concentrated under reduced pressure. After precipitation with pentane, the title compound was obtained as an off-white solid (0.305 g, 50%), gummy at 128° C.; ¹H NMR (300 MHz; CDCl₃) δ: 8.51 (1H, d), 7.87 (1H, s), 7.82 (2H, d), 7.40 (1H, d), 7.40-7.28 (3H, m), 7.22 (1H, d), 6.96 (1H, d), 6.89 (1H, d), 6.85 (1H, s), 4.97 (2H, s), 3.56 (3H, s), 2.36 (3H, s), 1.32 (9H, s); TOF MS ES⁺ exact mass calculated for C₂₉H₃₀N₆O: 479.2559(MH⁺). Found 479.2549(MH⁺).

### Example 107

### 2-{4-[4-(2-tert-Butyl-5-{6-methyl}pyridin-2-yl-1H-imidazol-4-yl)-pyridin-2-yl]-phenoxy}-acetamide

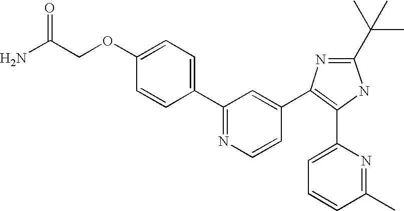

Intermediate 126 (0.5 g, 1.3 mmol) and 2-bromoacetamide (0.197 g, 1.43 mmol) were reacted as described for example 106 to afford the title compound as a white solid (0.347 g, 60.45%); m.p. 210° C.; TOF MS ES⁺ exact mass calculated for C₂₆H₂₇N₅O₂: 442.2243 (MH⁺). Found: 442.2221 (MH⁺).

### Example 108

### 4-(2-tert-Butyl-5-{6-methyl}-pyridin-2-yl-1H-imidazol-4-yl)-2-[4-(2-pyrrolidin-1-yl-ethoxy)-phenyl]-pyridine

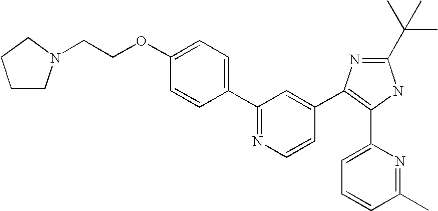

Intermediate 126 (0.4 g, 1.04 mmol) and 1-(2-chloroethyl)pyrrolidine hydrochloride (0.354 g, 2.08 mmol) were reacted as described for example 106 to afford the title compound as an off-white solid (0.12 g, 24%); m.p. 168° C.; TOF MS ES⁺ exact mass calculated for C₃₀H₃₅N₅O: 482.2920(MH⁺). Found: 482.2931 (MH⁺).

### Example 109

### 4-(2-tert-Butyl-5-pyridin-2-yl-1H-imidazol-4-yl)-2-[4-(1-methyl-1H-imidazol4-ylmethoxy)-phenyl]-pyridine

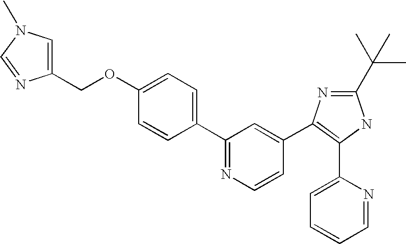

To a solution of example 83 (0.26 g , 0.67 mmol) in CH₂C₂ (40 ml) was added boron tribromide (2.1 ml, 2.1 mmol, 3.2 eq, solution 1M in CH₂Cl₂). The mixture was stirred at room temperature overnight. The reaction mixture was evaporated and neutralised with NaOH (1N), the resulting mixture was warmed up to 60° C. and stirred for 1 hour. After cooling to room temperature, the mixture was extracted with CH₂Cl₂. The aqueous phase was acidified with HCl (1N) and extracted with CH₂Cl₂. The organic phase was washed with NaHCO₃, dried over Na₂SO₄, and concentrated under reduced pressure to give 4-(4-{2-tert-Butyl-5-pyridin-2-yl-1H-imidazol-4-yl}-pyridin-2-yl]-phenol which was used without purification in the next step. A solution of 4-(4-{2-tert-Butyl-5-pyridin-2-yl-1H-imidazol-4-yl}-pyridin-2-yl]-phenol (0.14 g, 0.37 mmol) in acetone K₂CO₃ (0.156 g, 1.1 mmol) and intermediate 22 (0.094 g, 0.56 mmol) were heated at reflux for 2 days. The reaction mixture was filtered and the solvent was removed under reduced pressure. The residue was poured into water and extracted with CH₂Cl₂ . The organic phase was washed with water, dried over Na₂SO₄ and concentrated under reduced pressure. The crude product was purified by chromatography on silica gel (toluene/isopropylamine 90:10) to afford the title compound as a yellow solid (0.04 g, 23.3%); m.p. 156° C.; TOF MS ES⁺ exact mass calculated for C₂₈H₂₈N₆O: 465.2403 (MH⁺). Found: 465.2395 (MH⁺).

### Example 110

### 4-{2-Phenyl-5-[6-methyl]-pyridin-2-yl-1H-imidazol-4-yl}-2-(4-methanesulfonyl-phenyl)-pyridine

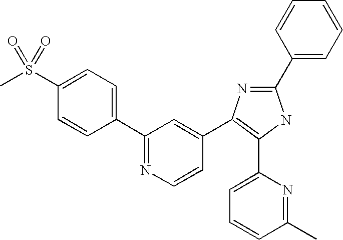

Intermediate 118 (6.5 g, 20.3 mmol) and benzaldehyde (4.3 ml, 40.6 mmol) were reacted as described for intermediate 119 to afford 2-phenyl-4-(pyridin-2-yl)-5-(2-bromo-pyridin-4-yl)-imidazole (4.5 g) which was used in the next step without purification. 2-Phenyl-4-(pyridin-2-yl)-5-(2-bromo-pyridin-4-yl)-imidazole (0.6 g, 1.53 mmol) and 4-(methanesulfonyl)phenyl boronic acid (0.338 g, 1.69 mmol) were reacted as described for example 81 to afford the title compound as a yellow powder (0.14 g, 19.63%); ¹H NMR (300 MHz; CDCl₃) δ: 8.67 (1H, d), 8.13-8.12 (2H, m), 7.96 (2H, d), 7.92 (1H, s), 7.68-7.26(8H, m), 7.02 (1H, d), 3.02 (3H, s), 2.54 (3H, s); TOF MS ES⁺ exact mass calculated for C₂₇H₂₂N₄O₂S: 467.1542 (MH⁺). Found: 467.1513 (MH⁺).

### Aminothiazole Examples

### Example 111

### 5-{2-4-(morpholin-4-yl)phenyl]pyridin-4-yl}-4-(pyridin-2-yl)-1,3-thiazol-2-amine

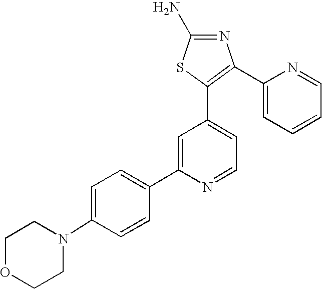

To a solution of intermediate 45 (0.4 g, 1.11 mmol) in CH₂Cl₂ (20 ml) was added polymer-supported pyridinium perbromide (0.62 g, 1 eq, 1.11 mmol) and the suspension shaken for 50 min. The resin was removed by filtration, with the filtrate being added directly to thiourea (0.25 g, 3 eq, 3.33 mmol) and the resin washed several times with ethanol. The filtrate was heated at reflux overnight,, allowed to cool and concentrated. The residue was basified with aqueous NaOH and extracted with CH₃Cl₂. The organic phase was washed with water, dried over Na₂SO₄, and concentrated under reduced pressure. After chromatography on silica gel (CH₂Cl₂/MeOH, 95:5 then 90:10) and crystallisation from ethyl acetate, the title compound was obtained as cream crystals (108 mg, 23.35%); m.p. 246° C.; [APCl MS] m/z 416 (MH⁺).

The following compounds of formula (IEf) were prepared by methods analogous to that described for Example 111 using the starting materials indicated (see Table 20).

TABLE 20

| | |
|---|---|
| | (IEf) |
| 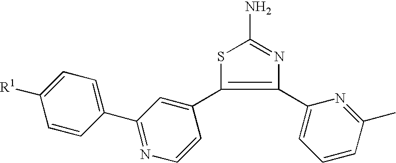 | |

| | | From | |
|---|---|---|---|
| Ex. | R¹ | Int. | Physical data |
| 112 | methanesulfonyl | 46 | TOF MS ES⁺ exact mass calculated for |
| | | | C₂₁H₁₈N₄O₂S₂ (MH+): 423.0949. |
| | | | Found: 423.0945; m.p. 236-238° C. |
| 113 | 4-ethylpiperazin- | 47 | TOF MS ES⁺ exact mass calculated for |
| | 1-yl | | C₂₆H₂₈N₆S (MH+): 457.2174. Found: |
| | | | 457.2213; m.p. 230-232° C. |
| 114 | morpholin-4-yl | 48 | TOF MS ES⁺ exact mass calculated for |
| | | | C₂₄H₂₃N₅OS (MH+): 430.1701. Found: |
| | | | 430.1698; m.p. 250-252° C. |
| 115 | (morpholin-4-yl)- | 57 | TOF MS ES⁺ exact mass calculated for |
| | carbonyl | | C₂₅H₂₃N₅O₂S (MH+): 458.1651. |
| | | | Found: 458.1602; m.p. 158-160° C. |
| 116 | (tetrahydropyran- | 58 | [APCI MS] m/z: 472 (MH+); m.p. 230- |
| | 4-yl)amino | | 232° C. |
| | carbonyl | | |
| 117 | (morpholin-4- | 59 | TOF MS ES⁺ exact mass calculated for |
| | yl)methyl | | C₂₅H₂₅N₅OS (MH+): 443.1860. Found: |
| | | | 443.1800; m.p. 190-192° C. |
| 118 | methoxy | 51 | [APCI MS] m/z: 375 (MH+); m.p. 188- |
| | | | 190° C. |
| 119 | trifluoromethoxy | 52 | [APCI MS] m/z: 429 (MH+); m.p. 222- |
| | | | 224° C. |
| 120 | aminocarbonyl | 54 | [APCI MS] m/z: 418 (MH+); m.p. 152- |
| | methoxy | | 154° C. |
| 121 | 2-(pyrolidin-1- | 53 | [APCI MS] m/z: 458 (MH+); m.p. 176- |
| | yl)ethoxy | | 178° C. |
| 122 | (1-methyl- | 55 | TOF MS ES⁺ exact mass calculated for |
| | imidazol-4- | | C₂₅H₂₂N₆OS (MH+): 455.1654. Found: |
| | yl)methoxy | | 455.1600; m.p. 226-228° C. |

The following compounds of formula (IEg) were prepared by methods analogous to that described for Example 111 using the starting materials indicated (see Table 21).

TABLE 21

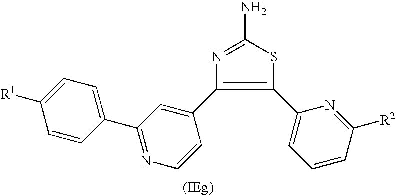

| | | | From | |
|---|---|---|---|---|
| Ex. | R¹ | R² | Int. | Physical data |
| 123 | (tetrahydropyran-4- | H | 65 | TOF MS ES⁺ exact mass calculated for |
| | yl)amino | | | C₂₅H₂₃N₅O₂S (MH+): 458.1651. Found: |
| | carbonyl | | | 458.1637; m.p. 268° C. |
| 124 | morpholin-4-yl | H | 66 | TOF MS ES⁺ exact mass calculated for |
| | | | | C₂₃H₂₁N₅OS (MH+): 416.1545. Found: |
| | | | | 416.1504; m.p. 276° C. |
| 125 | chloro | methyl | 67 | TOF MS ES⁺ exact mass calculated for |
| | | | | C₂₀H₁₅CIN₄S (MH+): 429.0997. |
| | | | | 379.0772; m.p. 222° C. |
| 126 | trifluoromethoxy | methyl | 68 | TOF MS ES⁺ exact mass calculated for |
| | | | | C₂₁H₁₅F₃N₄OS (MH+): 429.0997. |
| | | | | Found: 429.0958; m.p. 232° C. |
| 127 | ethanesulfonyl | methyl | 61 | TOF MS ES⁺ exact mass calculated for |
| | | | | C₂₂H₂₀N₄O₂S₂ (MH+): 437.1106. Found: |
| | | | | 437.1096; m.p. 219° C. |
| 128 | (tetrahydropyran-4- | methyl | 71 | TOF MS ES⁺ exact mass calculated for |
| | yl)aminocarbonyl | | | C₂₆H₂₅N₅O₂S (MH+): 472.1807. Found: |
| | | | | 472.1815; m.p. 283° C. |
| 129 | (morpholin-4- | methyl | 69 | TOF MS ES⁺ exact mass calculated for |
| | yl)carbonyl | | | C₂₅ H₂₃N₅O₂S (MH+): 458.1651. Found: |
| | | | | 458.1610; m.p. 246° C. |
| 130 | (4-ethylpiperazin-1- | methyl | 70 | TOF MS ES⁺ exact mass calculated for |
| | yl)carbonyl | | | C₂₇H₂₈N₆OS (MH+): 485.2123. Found: |
| | | | | 485.2128; m.p. 224° C. |
| 131 | (morpholin-4-yl)methyl | methyl | 77 | TOF MS ES⁺ exact mass calculated for |
| | | | | C₂₅H₂₅N₅OS (MH+): 444.1858. Found: |
| | | | | 444.1862; m.p. 236° C. |
| 132 | morpholin-4-yl | Methyl | 72 | TOF MS ES⁺ exact mass calculated for |
| | | | | C₂₄H₂₃N₅OS (MH+): 430.1701. Found: |
| | | | | 430.1648; m.p. 246° C. |
| 133 | 2-(pyrolidin-1-yl)ethoxy | methyl | 73 | TOF MS ES⁺ exact mass calculated for |
| | | | | C₂₆H₂₇N₅O₅ (MH+): 458.2014. Found: |
| | | | | 458.1963; m.p. 150° C. |
| 134 | aminocarbonylmethoxy | methyl | 74 | TOF MS ES⁺ exact mass calculated for |
| | | | | C₂₂H₁₉N₅O₂S (MH+): 418.1338. Found: |
| | | | | 418.1289; m.p. 191° C. |
| 135 | (morpholin-4- | methyl | 75 | TOF MS ES~ exact mass calculated for |
| | yl)carbonylmethoxy | | | C₂₆H₂₅N₅O₃₅ (MH+): 488.1756. Found: |
| | | | | 488.1700; m.p. 172° C. |
| 136 | (pyrolidin-1-yl)methyl | methyl | 78 | TOF MS ES⁺ exact mass calculated for |
| | | | | C₂₅H₂₅N₅S (MH+): 428.1909. Found: |
| | | | | 428.1861; m.p. 200° C. |
| 137 | (dimethylamino)methyl | methyl | 79 | TOF MS ES⁺ exact mass calculated for |
| | | | | C₂₃H₂₃N₅₅ (MH+): 402.1752. Found: |
| | | | | 402.1707;m.p. 210° C. |

### Examples 138 to 140

Step 1: Intermediate 128 supported on resin (1 g) was weighed out into a peptide vessel. Then 4-formylphenylboronic acid (870 mg, 5.8 mmol, 10 eq), Pd(PPh₃)₄ (134 mg, 0.16 mmol, 0.2 eq), and sodium carbonate (615 mg, 5.8 mmol, 2M) were added and suspended in toluene/EtOH (8:2, 20 mL). The reaction vessel was purged with argon for 5 min, and the mixture was stirred at 90° C. for 16 h. The resin was washed with DMF (3×10 mL), water (3×10 mL), EtOH (3×10 mL) and CH₂Cl₂ (3×10 mL).

Step 2: The product from step 1 was placed into a peptide vessel with a solution of NHR⁵R⁶ (5.8 mmol, 10 eq) in trimethylorthoformate (5.4 mL). Then a solution of sodium cyanoborohydride (0.2M) in THF (5.4 mL) with acetic acid (110 μL) was added. The reaction vessel was purged with argon for 5 min and the mixture was stirred at 60° C. for 16 h. The resin was washed with DMF (3×10 mL), EtOH (3×10 mL) and CH₂Cl₂ (3×10 mL). The resin was treated with a solution of 20% TFA in CH₂Cl₂ and the solvent was removed under reduced pressure. Purification of the residue by HPLC chromatography (water/acetonitrile gradient) gave the products of formula (IEh) shown in Table 22.

TABLE 22

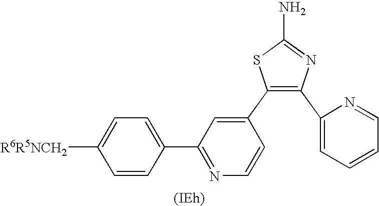

| Ex | R⁵ | R⁶ | Physical data |
|---|---|---|---|
| 138 | H | isopropyl | [APCI MS] m/z 402 MH+ |
| 139 | —(CH₂)₄— | | TOF MS ES⁺ exact mass calculated for |
| | | | C₂₄H₂₃N₅S (MH+): 414.1752. Found 414.1766. |
| 140 | H | cyclobutyl | TOF MS ES⁺ exact mass calculated for |
| | | | C₂₄H₂₃N₅S (MH+): 414.1752. Found 414.1749. |

### Examples 141 to 144

Step 1: Intermediate 128 or intermediate 129 supported on resin (1 g) were weighed out into a peptide vessel. Then 4-hydroxyphenylboronic acid (800 mg, 5.8 mmol, 10 eq), Pd(PPh₃)₄ (134 mg, 0.16 mmol, 0.2 eq), and sodium carbonate (615 mg, 5.8 mmol, 2M) were added and suspended in toluene/EtOH (8:2, 20 mL).The reaction vessel was purged with argon for 5 min, and the mixture was stirred at 90° C. for 16 h. The resin was washed with DMF (3×10 mL), water (3×10 mL), EtOH (3×10 mL) and CH₂Cl₂ (3×10 mL).

Step 2: The product from step 1 was placed into a peptide vessel with a solution of R—Cl (5.8 mmol, 10 eq) in DMSO (10 mL). Then a solution of potassium carbonate (802 mg, 5.8 mmol, 10 eq) in DMSO (5 mL) was added. The reaction vessel was purged with argon for 5 min and the mixture was stirred at 90° C. for 16 h. The resin was washed with DMF (3×10 mL), EtOH (3×10 mL) and CH₂Cl₂ (3×10 mL). The resin was treated with a solution of 20% TFA in CH₂Cl₂ and the solvent was removed under reduced pressure. Purification of the residue by HPLC chromatography (water/acetonitrile gradient) gave the products of formula (IEj) shown in Table 23

TABLE 23

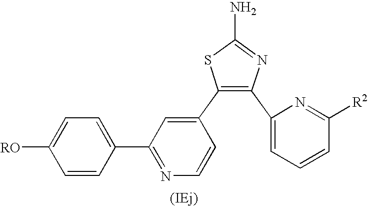

| | | | From intermediate | |
|---|---|---|---|---|
| Ex | R | R² | Supported on resin | Physical data |
| 141 | 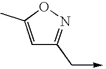 | methyl | 55 | TOF MS ES⁺ exact mass calculated for C₂₅H₂₁N₅O₂S (MH+): 456.1494. Found: 456.1457. |
| 142 | 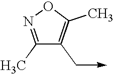 | H | 54 | TOF MS ES⁺ exact mass calculated for C₂₅H₂₁N₅O₂S (MH+): 456.1494. Found: 456:1545. |
| 143 | 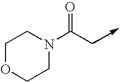 | methyl | 55 | TOF MS ES⁺ exact mass calculated for C₂₆H₂₅N₅O₃S (MH+): 488.1756. Found: 488.1792. |
| 144 | 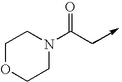 | H | 54 | TOF MS ES⁺ exact mass calculated for C₂₅H₂₃N₅O₃S (MH+): 474.1600. Found: 474.1552. |

### Examples 145 to 156

Step 1: Intermediate 129 supported on resin (1 g) was weighed out into a peptide vessel. Then 4-methoxycarbonylphenylboronic acid (1.05 g, 5.8 mmol, 10 eq), Pd(PPh₃)₄ (0.134 g, 0.16 mmol, 0.2 eq), and a aqueous solution of sodium carbonate (0.615 g, 5.8 mmol, 2M) were added and suspended in toluene/EtOH (8:2, 20 mL). The reaction vessel was purged with argon for 5 min, and the mixture was stirred at 90° C. for 16 h. The resin was washed with DMF (3×10 mL), water (3×10 mL), EtOH (3×10 mL) and CH₂Cl₂ (3×10 mL). Then resin was added to a sodium hydroxide solution (2M) in dioxane (10 mL). The reaction mixture was stirred at 50° C. for 16 h. The resin was washed with DMF (3×10 mL), EtOH (3×10 mL) and CH₂Cl₂ (3×10 mL).

Step 2: The product from step 1 was placed into a peptide vessel with a solution of NHR⁵R⁶ (5.8 mmol, 10 eq) in DMF (5 mL). Then a solution of HOBT (1.18 g, 8.7 mmol, 15 eq) and EDCl (1.36 mL, 8.7 mmol, 15 eq) in DMF (5 mL) was added. The reaction vessel was purged with argon for 5 min and the mixture was stirred at 70° C. for 16 h. The resin was washed with DMF (3×10 mL), EtOH (3×10 mL), CH₂Cl₂ (3×10 mL). The resin was treated with a solution of 20% TFA in CH₂Cl₂ and the solvent was removed under reduced pressure. Purification of the residue by HPLC chromatography (water/acetonitrile gradient) gave the products of formula (IEk) shown in Table 24.

TABLE 24

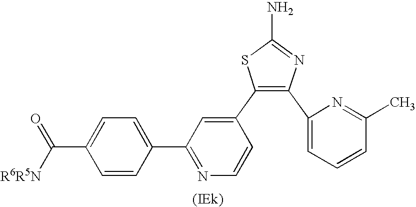

| Ex | R⁶R⁵NCO— | Physical data |
|---|---|---|
| 145 | 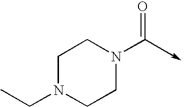 | TOF MS ES⁺ exact mass calculated for C₂₇H₂₈N₆OS (MH+): 485.2123. Found: 485.2123. |
| 146 | 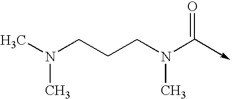 | TOF MS ES⁺ exact mass calculated for C₂₇H₃₀N₆OS (MH+): 487.2280. Found: 487.2267. |
| 147 | 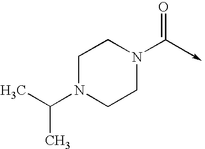 | TOF MS ES⁺ exact mass calculated for C₂₈H₃₀N₆OS (MH+): 499.2280. Found: 499.2277. |
| 148 | 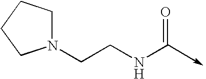 | TOF MS ES⁺ exact mass calculated for C₂₇H₂₈N₆OS (MH+): 485.2123. Found: 485.2094. |
| 149 | 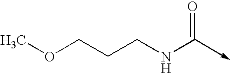 | TOF MS ES⁺ exact mass calculated for C₂₅H₂₅N₆O₂S (MH+): 460.1807. Found: 460.1810. |
| 150 | 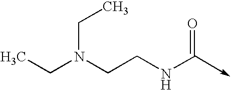 | TOF MS ES⁺ exact mass calculated for C₂₇H₃₀N₆OS (MH+): 487.2280. Found: 487.2260. |
| 151 | 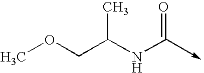 | TOF MS ES⁺ exact mass calculated for C₂₅H₂₅N₅O₂S (MH+): 460.1807. Found: 460.1799. |
| 152 | 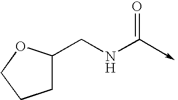 | TOF MS ES⁺ exact mass calculated for C₂₆H₂₅N₅O₂S (MH+): 472.1807. Found: 472.1798. |
| 153 | 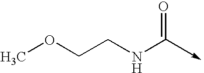 | TOF MS ES⁺ exact mass calculated for C₂₄H₂₃N₅O₂S (MH+): 466.1651. Found: 466.1633. |
| 154 | 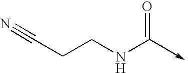 | TOF MS ES⁺ exact mass calculated for C₂₅H₂₂N₆OS (MH+): 455.1654. Found: 455.1626. |
| 155 | 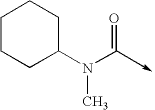 | TOF MS ES⁺ exact mass calculated for C₂₈H₂₉N₅OS (MH+): 484.2171. Found: 484.2133. |
| 156 | 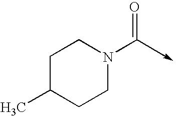 | TOF MS ES⁺ exact mass calculated for C₂₇H₂₇N₅OS (MH+): 470.2014. Found: 470.1964. |

Biology

The biological activity of the compounds of the invention may be assessed using the following assays:

Assay 1 (Cellular Transcriptional Assay)

The potential for compounds of the invention to inhibit TGF-β signalling may be demonstrated, for example, using the following in vitro assay.

The assay was performed in HepG2 cells stably transfected with the PAI-1 promoter (known to be a strong TGF-β responsive promoter) linked to a luciferase (firefly) reporter gene. The compounds were selected on their ability to inhibit luciferase activity in cells exposed to TGF-β. In addition, cells were transfected with a second luciferase (Renilla) gene which was not driven by a TGF-β responsive promoter and was used as a toxicity control.

96 well microplates were seeded, using a multidrop apparatus, with the stably transfected cell line at a concentration of 35000 cells per well in 200 μl of serum-containing medium. These plates were placed in a cell incubator.

18 to 24 hours later (Day 2), cell-incubation procedure was launched. Cells were incubated with TGF-β and a candidate compound at concentrations in the range 50 nM to 10 μM (final concentration of DMSO 1%). The final concentration of TGF-β (rhTGFβ-1) used in the test was 1 ng/mL. Cells were incubated with a candidate compound 15-30 mins prior to the addition of TGF-β. The final volume of the test reaction was 150 μl. Each well contained only one candidate compound and its effect on the PAI-1 promoter was monitored.

Columns 11 and 12 were employed as controls. Column 11 contained 8 wells in which the cells were incubated in the presence of TGF-β, without a candidate compound. Column 11 was used to determine the ‘reference TGF-β induced firefly luciferase value’ against which values measured in the test wells (to quantify inhibitory activity) were compared. In wells A12 to D12, cells were grown in medium without TGF-β. The firefly luciferase values obtained from these positions are representative of the ‘basal firefly luciferase activity’. In wells E12 to H12, cells were incubated in the presence of TGF-β and 500 μM CPO (Cyclopentenone, Sigma), a cell toxic compound. The toxicity was revealed by decreased firefly and renilla luciferase activities (around 50% of those obtained in column 11).

12 to 18 hours later (day 3), the luciferase quantification procedure was launched. The following reactions were performed using reagents obtained from a Dual Luciferase Assay Kit (Promega). Cells were washed and lysed with the addition of 10 μl of passive lysis buffer (Promega). Following agitation (15 to 30 mins), luciferase activities of the plates were read in a dual-injector luminometer (BMG lumistar). For this purpose, 50 μl of luciferase assay reagent and 50 μl of ‘Stop & Glo’ buffer were injected sequentially to quantify the activities of both luciferases. Data obtained from the measurements were processed and analysed using suitable software. The mean Luciferase activity value obtained in wells A11 to H11 (Column 11, TGF-βonly) was considered to represent 100% and values obtained in wells A12 to D12 (cells in medium alone) gave a basal level (0%). For each of the compounds tested, a concentration response curve was constructed from which an IC₅₀ value was determined graphically.

Assay 2 (Alk5 Fluorescence Polarization Assay)

Kinase inhibitor compounds conjugated to fluorophores, can be used as fluorescent ligands to monitor ATP competitive binding of other compounds to a given kinase. The increase in depolarization of plane polarized light, caused by release of the bound ligand into solution, is measured as a polarization/anisotropy value. This protocol details the use of a rhodamine green-labelled ligand for assays using recombinant GST-ALK5 (residues 198-503).

Assay buffer components: 62.5 mM Hepes pH 7.5 (Sigma H-4034), 1 mM DTT (Sigma D-0632),12.5 mM MgCl₂ (Sigma M-9272),1.25 mM CHAPS (Sigma C-3023).

Protocol: Solid compound stocks were dissolved in 100% DMSO to a concentration of 1 mM and transferred into column 1, rows A-H of a 96-well, U bottom, polypropylene plate (Costar #3365) to make a compound plate. The compounds were serially diluted (3-fold in 100% DMSO) across the plate to column 11 to yield 11 concentrations for each test compound. Column 12 contained only DMSO. A Rapidplate™-96 was used to transfer 1 μl of sample from each well into a 96-well, black, U-bottom, non-treated plate (Costar #3792) to create an assay plate.

ALK5 was added to assay buffer containing the above components and 1 nM of the rhodamine green-labelled ligand so that the final ALK5 concentration was 10 nM based on active site titration of the enzyme. The enzyme/ligand reagent (39 μl) was added to each well of the previously prepared assay plates. A control compound (1 μl) was added to column 12, rows E-H for the low control values. The plates were read immediately on a LJL Acquest fluorescence reader (Molecular Devices, serial number AQ1048) with excitation, emission, and dichroic filters of 485 nm, 530 nm, and 505 nm, respectively. The fluorescence polarization for each well was calculated by the Acquest reader and then imported into curve fitting software for construction of concentration response curves. The normalized response was determined relative to the high controls (1 μl DMSO in column 12, rows A-D) and the low controls (1 μl of control compound in column 12, rows E-H). An IC₅₀ value was then calculated for each compound

Using the above assays all Examples of the invention show ALK5 receptor modulator activity (having IC₅₀ values in the range of 0.4 to 275 nM) and TGF-β cellular activity (having IC₅₀ values in the range of 0.001 to 10 μM).

4-{4-[4-(2-tert-Butyl-5-{6-methyl}-pyridin-2-yl-1 H-imidazol-4-yl)-pyridin-2-yl]-phenyl}-morpholine (Example 86) showed an ALK5 receptor modulator activity of 34 nM and TGF-β cellular activity of 183 nM.

N-(tetrahydropyran-4-yl)-4-(4-{2-isopropyl-5-[6-methyl-pyridin-2-yl]-1H-imidazol-4-yl}-pyridin-2-yl)-benzamide (Example 96) showed an ALK5 receptor modulator activity of 25 nM and TGF-β cellular activity of <14 nM.

1. A compound of formula (I), a pharmaceutically acceptable salt, solvate or derivative thereof: 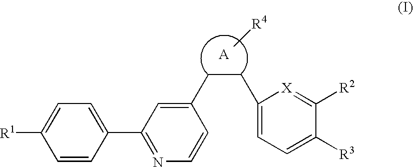
wherein
A is furan, dioxolane, thiophene, pyrrole, imidazole, pyrrolidine, pyran, pyridine, pyrimidine, morpholine, piperidine, oxazole, isoxazole, oxazoline, oxazolidine, thiazole, isothiazole, thiadiazole, benzofuran, indole, isoindole, indazole, imidazopyridine, quinazoline, quinoline, isoquinoline, pyrazole or triazole;
X is N or CH;
R¹ is hydrogen, C₁₋₆alkyl, C₁₋₆alkenyl, C₁₋₆alkoxy, halo, cyano, perfluoro C₁₋₆alkyl, perfluoroC₁₋₆alkoxy, —NR⁵R⁶, —(CH₂)ₙNR⁵R⁶, —O(CH₂)ₙOR⁷, —O(CH₂)ₙ-Het, —O(CH₂)ₙNR⁵R⁶, —CONR⁵R⁶, —CO(CH₂)ₙNR⁵R⁶, —SO₂R⁷, —SO₂NR⁵R⁶, —NR⁵SO₂R⁷, —NR⁵COR⁷, —O(CH₂)ₙCONR⁵R⁶, —NR⁵CO(CH₂)ₙNR⁵R⁶ or —C(O)R⁷;
R² is hydrogen, C₁₋₆alkyl, halo, cyano or perfluoroC₁₋₆alkyl;
R³ is hydrogen or halo;
R⁴ is hydrogen, halo, phenyl, C₁₋₆alkyl or —NR⁵R⁶;
where
R⁵ and R⁶ are independently selected from hydrogen; Het; C₃₋₆cycloalkyl optionally substituted by C₁₋₆alkyl; or by C₁₋₆alkyl optionally substituted by Het, alkoxy, cyano or —NR^{a}R^{b} (where R^{a} and R^{b} which may the same or different are hydrogen or C₁₋₆alkyl, or R^{a} and R^{b} together with the nitrogen atom to which they are attached may form a 4,5 or 6-membered saturated ring); or R⁵ and R⁶ together with the nitrogen atom to which they are attached form a 3, 4, 5, 6 or 7-membered saturated or unsaturated ring which may contain one or more heteroatoms selected from N, S or O, and wherein the ring may be further substituted by one or more substituents selected from halo (such as fluoro, chloro, bromo), cyano, —CF₃, hydroxy, —OCF₃, C₁₋₆alkyl and C₁₋₆alkoxy;
R⁷ is selected from hydrogen and C₁₋₆alkyl;
Het is a 5 or 6-membered C-linked heterocyclyl group which may be saturated, unsaturated or aromatic, which may contain one or more heteroatoms selected from N, S or O and which may be substituted by C₁₋₆alkyl; and
n is 1-4;
with the provisos that:
a) when A is thiazole (wherein the thiazole sulfur is on the same side as the 4-pyridyl moiety); X is N; R¹ is hydrogen, C₁₋₆alkyl, C₁₋₆alkoxy, halo, cyano, perfluoroC₁₋₆alkyl or perfluoroC₁₋₆alkoxy; R² is hydrogen, C₁₋₆alkyl, halo, cyano or perfluoroC₁₋₆alkyl; and R³ is hydrogen or halo; then R⁴ is not NH₂; and
b) when X is N, A is pyrazole (where the ring containing X is attached to the pyrazole ring at carbon atom next to a pyrazole ring nitrogen), R² is hydrogen then R³ is not hydrogen.

2. A compound according to claim 1 wherein A is imidazole optionally substituted by one R⁴ substitutent.

3. A compound according to claim 1 wherein X is N.

4. A compound according to claim 1 wherein R¹ is C₁₋₆alkyl, C₁₋₆alkoxy, halo, cyano, perfluoroC₁₋₆alkoxy, —NR⁵R⁶, —(CH₂)ₙNR⁵R⁶, —O(CH₂)ₙOR⁷, —O(CH₂)ₙ-Het, —O(CH₂)ₙNR⁵R⁶ —CONR⁵R⁶, —SO₂R⁷, —NR⁵SO₂R⁷, —NR⁵COR⁷, —O(CH₂)ₙCONR⁵R⁶, —NR⁵CO(CH₂)ₙNR⁵R⁶ or —C(O)R⁷.

5. A compound according to claim 1 wherein R² is hydrogen, C₁₋₆alkyl or fluoro.

6. A compound according to claim 1 wherein R³ is hydrogen.

7. A compound according to claim 1 wherein R⁴ is hydrogen, phenyl, C₁₋₆alkyl or halo.

8. A compound according to claim 1 wherein R⁵ and R⁶ are independently selected from hydrogen; Het; C₃₋₆cycloalkyl optionally substituted by C₁₋₆alkyl; or by C₁₋₆alkyl optionally substituted by Het, alkoxy, cyano or —NR^{a}R^{b} (where R^{a} and R^{b} which may the same or different are hydrogen or C₁₋₆alkyl, or R^{a} and R^{b} together with the nitrogen atom to which they are attached may form a 4, 5 or 6-membered saturated ring); or R⁵ and R⁶ together with the atom to which they are attached form a morpholine, piperidine, pyrrolidine or piperazine ring, each of which may be substituted by halo (such as fluoro, chloro, bromo), cyano, —CF₃, hydroxy, —OCF₃, C₁₋₄alkyl or C₁₋₄alkoxy.

9. A compound according to claim 1 wherein
A is imidazole;
X is N;
R¹ is C₁₋₆alkyl, C₁₋₆alkoxy, halo, cyano, perfluoroC₁₋₆alkoxy, —NR⁵R⁶, —(CH₂)ₙNR⁵R⁶, —(CH₂)ₙOR⁷, —O(CH₂)ₙ-Het, —O(CH₂)ₙNR⁵R⁶, —CONR⁵R⁶, —SO₂R⁷, —NR⁵SO₂R⁷, —R⁵COR⁷, —O(CH₂)ₙCONR⁵R⁶, —NR⁵CO(CH₂)ₙNR⁵R⁶ or —C(O)R⁷;
R² is hydrogen, C₁₋₆alkyl or fluoro;
R³ is hydrogen or halo;
R⁴ is hydrogen, phenyl, C₁₋₆alkyl or halo;
R⁵ and R⁶ are independently selected from hydrogen, Het or C₁₋₆alkyl; or R⁵ and R⁶ together with the atom to which they are attached form a morpholine, piperidine, pyrrolidine or piperazine ring, each of which may be substituted by halo (such as fluoro, chloro, bromo), cyano, —CF₃, hydroxy, —OCF₃, C₁₋₄alkyl or C₁₋₄alkoxy;
R⁷ is selected from hydrogen and C₁₋₆alkyl;
Het is a 5 or 6-membered C-linked heterocyclyl group which may be saturated, unsaturated or aromatic, which may contain one or more heteroatoms selected from N, S or O and which may be substituted by C₁₋₆alkyl; and n is 1-4.

10. A compound according to claim 1 wherein the compound is selected from the list:
4-{2-tert-Butyl-5-[6-methyl]-pyridin-2-yl-1H-imidazol-4-yl}-2-(4-methanesulfonyl-phenyl)-pyridine (Example 84);
4-{4-[4-(2-tert-Butyl-5-{6-methyl}-pyridin-2-yl-1H-imidazol-4-yl)-pyridin-2-yl]-phenyl}-morpholine (Example 86);
N-(tetrahydropyran-4-yl)-4-(4-{2-isopropyl-5-[6-methyl-pyridin-2-yl]-1H-imidazol-4-yl}-pyridin-2-yl)-benzamide (Example 96);
4-{4-[4-(2-isopropyl-5- {6-methyl}-pyridin-2-yl-1H-imidazol-4-yl)-pyridin-2-yl]-phenyl}-morpholine (Example 97);
4-(4-{4-[2-Isopropyl-5-(6-methyl-pyridin-2-yl)-1H-imidazol-4-yl]-pyridin-2-yl}-benzyl)-dimethyl-amine (Example 105);
4-(4-{4-[2-Isopropyl-5-(6-methyl-pyridin-2-yl)-1H-imidazol-4-yl]-pyridin-2-yl}-benzyl)-morpholine (Example 104);
N-(tetrahydropyran-4-yl)-4-(4-{2-tert-Butyl-5-[6-methyl-pyridin-2-yl]-1H-imidazol-4-yl}-pyridin-2-yl)-benzamide (Example 81);
(4-{4-[2-tert-Butyl-5-(6-methyl-pyridin-2-yl)-1H-imidazol-4-yl]-pyridin-2-yl}-benzyl)-pyrrolidine (Example 103);
4-(2-tert-Butyl-5-{6-methyl}-pyridin-2-yl-1H-imidazol-4-yl)-2-[4-(2-pyrrolidin-1-yl-ethoxy)-phenyl]-pyridine (Example 108); and
4-{4-[4-(2-methyl-5-{6-methyl}-pyridin-2-yl-1H-imidazol-4-yl)-pyridin-2-yl]-phenyl}-morpholine (Example 98);
and pharmaceutically acceptable salts, solvates and derivatives thereof.

11. A pharmaceutical composition comprising a compound defined in claim 1 and a pharmaceutically acceptable carrier or diluent.

12-15. (canceled)

16. A method for the treatment or prophylaxis of a disorder mediated by the ALK5 receptor in mammals, wherein the disorder is selected from chronic renal disease, acute renal disease, wound healing, arthritis, osteoporosis, kidney disease, congestive heart failure, ulcers, ocular disorders, corneal wounds, diabetic nephropathy, impaired neurological function, Alzheimer's disease, atherosclerosis, peritoneal and sub-dermal adhesion, any disease wherein fibrosis is a major component, including, but not limited to lung fibrosis, kidney fibrosis, liver fibrosis [for example, hepatitis B virus (HBV), hepatitis C virus (HCV)], alcohol induced hepatitis, retroperitoneal fibrosis, mesenteric fibrosis, haemochromatosis and primary biliary cirrhosis, endometriosis, keloids and restenosis, which method comprises administering to a mammal in need of such treatment or prophylaxis, a compound of formula I.
